SEQUENCE LISTING (56 sequences)

SEQ ID NO: 1 - protein, 633 aa, Artificial Sequence
  Synthetic
  MISC_FEATURE: GP  DeltaTM-X-HIS protein
MEFWLSWVFLVAILKGVQCVPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVSNGAKNISGQSPARTSSDPGTNTTTEDHKIMASENSSAMVQVHSQGREAAVSHLTTLATISTSPQSLTTKPGPDNSTHNTPVYKLDISEATQVEQHHRRTDNDSTASDTPSATTAAGPPKAENTNTSKSTDFLDPATTTSPQNHSETAGNNNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPIEGRHHHHHH

SEQ ID NO: 2 - DNA, 1905 bp, Artificial Sequence
  Synthetic
  misc_feature: GP DeltaTM-X-HIS DNA
atggagttctggttgtcttgggtgtttctcgtcgctattctgaagggggtgcagtgcgtgccactgggagtgatccacaacagcactctccaagtctccgacgtggacaagctcgtgtgccgagacaagctttccagcacaaaccagctgcggtccgtgggactgaacctggagggaaatggcgtggccaccgatgtgccgtctgcaaccaagagatggggcttccgctcgggcgtgcccccgaaagtggtcaactacgaggccggagaatgggccgaaaactgttacaacctggagatcaagaagcccgacggttccgaatgcctccctgccgcccctgacggaatcaggggattcccgagatgccgctacgtgcacaaagtgtccgggactggaccttgtgctggagatttcgccttccacaaggagggcgcctttttcctgtacgaccggctcgcatccaccgtgatctatagaggaaccaccttcgcggaaggagtggtcgccttcctgatcctgccgcaagccaagaaggacttctttagctcccatccactgcgcgagcccgtgaatgccaccgaagatccctcgtccggctattactccacaaccatccggtaccaagccaccgggttcggtaccaacgagactgagtacctgttcgaagtcgacaacctgacttacgtgcagctcgaatcgcggttcacgccacagttcctcctgcaactcaacgaaactatctacactagcgggaaacgctcgaacaccaccgggaagctgatttggaaggtcaaccctgaaatcgacaccaccatcggagagtgggctttttgggagactaagaagaacctcacccggaagatcagatccgaggaattgtccttcactgtggtgtccaacggagcgaagaacatcagcggacagtcccccgcacggacctcatcggacccgggaaccaacaccaccaccgaggaccacaagatcatggcctcggagaactcatccgccatggtgcaagtgcatagccagggccgcgaagccgccgtgtcccacctgactaccctggcgaccatcagcaccagcccgcagtcgctgactactaagcctgggccagacaacagcacccacaacacccctgtgtacaagctggacatttccgaagctactcaggtcgagcagcatcaccggcggaccgacaacgattccaccgcttccgacaccccctccgccaccaccgccgccggcccgccgaaggccgaaaacaccaatacgtcaaagtcgaccgatttccttgaccccgccaccactacgagcccccagaaccactcagaaactgcgggcaacaacaacacccaccaccaggatactggagaggagtccgcatcaagcggaaagctcggcctgattactaacactatcgcgggggtcgctggattgattaccggtggtagaaggaccaggagagaggccattgtgaacgcgcagccgaagtgcaaccctaatctgcattactggaccactcaggacgagggcgccgccatcggcctggcctggattccgtacttcggcccggccgcggaaggcatatacattgagggacttatgcacaatcaggatggactgatttgtggtctgcgccagctggccaacgaaaccacccaggcgctgcagctgttcctccgggcaaccaccgaacttcgcactttctccatcctgaaccggaaggccattgacttcctcttgcaacgctggggaggaacttgccacatcctgggtcctgattgctgcatcgaaccgcatgactggacaaagaacatcaccgacaaaatcgaccagatcatccacgatttcgtggacaagaccctgcctatcgagggccggcaccatcaccaccaccattagtga

SEQ ID NO: 3 - protein, 482 aa, Artificial Sequence
  Synthetic
  MISC_FEATURE: GP DeltaTM-DeltaMUC-X-HIS protein
MEFWLSWVFLVAILKGVQCVPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPIEGRHHHHHH

SEQ ID NO: 4 - DNA, 1452 bp, Artificial Sequence
  Synthetic
  misc_feature: GP DeltaTM-DeltaMUC-X-HIS DNA
atggagttctggttgtcttgggtgtttctcgtcgctattctgaagggggtgcagtgcgtgccactgggagtgatccacaacagcactctccaagtctccgacgtggacaagctcgtgtgccgagacaagctttccagcacaaaccagctgcggtccgtgggactgaacctggagggaaatggcgtggccaccgatgtgccgtctgcaaccaagagatggggcttccgctcgggcgtgcccccgaaagtggtcaactacgaggccggagaatgggccgaaaactgttacaacctggagatcaagaagcccgacggttccgaatgcctccctgccgcccctgacggaatcaggggattcccgagatgccgctacgtgcacaaagtgtccgggactggaccttgtgctggagatttcgccttccacaaggagggcgcctttttcctgtacgaccggctcgcatccaccgtgatctatagaggaaccaccttcgcggaaggagtggtcgccttcctgatcctgccgcaagccaagaaggacttctttagctcccatccactgcgcgagcccgtgaatgccaccgaagatccctcgtccggctattactccacaaccatccggtaccaagccaccgggttcggtaccaacgagactgagtacctgttcgaagtcgacaacctgacttacgtgcagctcgaatcgcggttcacgccacagttcctcctgcaactcaacgaaactatctacactagcgggaaacgctcgaacaccaccgggaagctgatttggaaggtcaaccctgaaatcgacaccaccatcggagagtgggctttttgggagactaagaagaacctcacccggaagatcagatccgaggaattgtccttcaccgtggtcaacacccaccaccaggatactggagaggagtccgcatcaagcggaaagctcggcctgattactaacactatcgcgggggtcgctggattgattaccggtggtagaaggaccaggagagaggccattgtgaacgcgcagccgaagtgcaaccctaatctgcattactggaccactcaggacgagggcgccgccatcggcctggcctggattccgtacttcggcccggccgcggaaggcatatacattgagggacttatgcacaatcaggatggactgatttgtggtctgcgccagctggccaacgaaaccacccaggcgctgcagctgttcctccgggcaaccaccgaacttcgcactttctccatcctgaaccggaaggccattgacttcctcttgcaacgctggggaggaacttgccacatcctgggtcctgattgctgcatcgaaccgcatgactggacaaagaacatcaccgacaaaatcgaccagatcatccacgatttcgtggacaagaccctgcctatcgagggccggcaccatcaccaccaccattagtga

SEQ ID NO: 5 - protein, 513 aa, Artificial Sequence
  Synthetic
  MISC_FEATURE: GP DeltaTM-DeltaMUC-X-T4-HIS protein
MEFWLSWVFLVAILKGVQCVPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPIEGRGSGYIPEAPRDGQAYVRKDGEWVLLSTFLGTHHHHHH

SEQ ID NO: 6 - DNA, 1545 bp, Artificial Sequence
  Synthetic
  misc_feature: GP DeltaTM-DeltaMUC-X-T4-HIS DNA
atggagttctggttgtcttgggtgtttctcgtcgctattctgaagggggtgcagtgcgtgccactgggagtgatccacaacagcactctccaagtctccgacgtggacaagctcgtgtgccgagacaagctttccagcacaaaccagctgcggtccgtgggactgaacctggagggaaatggcgtggccaccgatgtgccgtctgcaaccaagagatggggcttccgctcgggcgtgcccccgaaagtggtcaactacgaggccggagaatgggccgaaaactgttacaacctggagatcaagaagcccgacggttccgaatgcctccctgccgcccctgacggaatcaggggattcccgagatgccgctacgtgcacaaagtgtccgggactggaccttgtgctggagatttcgccttccacaaggagggcgcctttttcctgtacgaccggctcgcatccaccgtgatctatagaggaaccaccttcgcggaaggagtggtcgccttcctgatcctgccgcaagccaagaaggacttctttagctcccatccactgcgcgagcccgtgaatgccaccgaagatccctcgtccggctattactccacaaccatccggtaccaagccaccgggttcggtaccaacgagactgagtacctgttcgaagtcgacaacctgacttacgtgcagctcgaatcgcggttcacgccacagttcctcctgcaactcaacgaaactatctacactagcgggaaacgctcgaacaccaccgggaagctgatttggaaggtcaaccctgaaatcgacaccaccatcggagagtgggctttttgggagactaagaagaacctcacccggaagatcagatccgaggaattgtccttcaccgtggtcaacacccaccaccaggatactggagaggagtccgcatcaagcggaaagctcggcctgattactaacactatcgcgggggtcgctggattgattaccggtggtagaaggaccaggagagaggccattgtgaacgcgcagccgaagtgcaaccctaatctgcattactggaccactcaggacgagggcgccgccatcggcctggcctggattccgtacttcggcccggccgcggaaggcatatacattgagggacttatgcacaatcaggatggactgatttgtggtctgcgccagctggccaacgaaaccacccaggcgctgcagctgttcctccgggcaaccaccgaacttcgcactttctccatcctgaaccggaaggccattgacttcctcttgcaacgctggggaggaacttgccacatcctgggtcctgattgctgcatcgaaccgcatgactggacaaagaacatcaccgacaaaatcgaccagatcatccacgatttcgtggacaagaccctgcctatcgaagggcgcggatcaggttacatccccgaggccccgagagacggccaggcctacgtgcggaaggacggcgaatgggtcctcctgtccaccttccttgggactcaccatcaccaccaccattgataa

SEQ ID NO: 7 - protein, 514 aa, Artificial Sequence
  Synthetic
  MISC_FEATURE: GP DeltaTM-DeltaMUC-X-GCN4-HIS protein
MEFWLSWVFLVAILKGVQCVPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPIEGRGSGKQIEDKIEEILSKIYHIENEIARIKKLIGHHHHHH

SEQ ID NO: 8 - DNA, 1548 bp, Artificial Sequence
  Synthetic
  misc_feature: GP DeltaTM-DeltaMUC-X-GCN4-HIS DNA
atggagttctggttgtcttgggtgtttctcgtcgctattctgaagggggtgcagtgcgtgccactgggagtgatccacaacagcactctccaagtctccgacgtggacaagctcgtgtgccgagacaagctttccagcacaaaccagctgcggtccgtgggactgaacctggagggaaatggcgtggccaccgatgtgccgtctgcaaccaagagatggggcttccgctcgggcgtgcccccgaaagtggtcaactacgaggccggagaatgggccgaaaactgttacaacctggagatcaagaagcccgacggttccgaatgcctccctgccgcccctgacggaatcaggggattcccgagatgccgctacgtgcacaaagtgtccgggactggaccttgtgctggagatttcgccttccacaaggagggcgcctttttcctgtacgaccggctcgcatccaccgtgatctatagaggaaccaccttcgcggaaggagtggtcgccttcctgatcctgccgcaagccaagaaggacttctttagctcccatccactgcgcgagcccgtgaatgccaccgaagatccctcgtccggctattactccacaaccatccggtaccaagccaccgggttcggtaccaacgagactgagtacctgttcgaagtcgacaacctgacttacgtgcagctcgaatcgcggttcacgccacagttcctcctgcaactcaacgaaactatctacactagcgggaaacgctcgaacaccaccgggaagctgatttggaaggtcaaccctgaaatcgacaccaccatcggagagtgggctttttgggagactaagaagaacctcacccggaagatcagatccgaggaattgtccttcaccgtggtcaacacccaccaccaggatactggagaggagtccgcatcaagcggaaagctcggcctgattactaacactatcgcgggggtcgctggattgattaccggtggtagaaggaccaggagagaggccattgtgaacgcgcagccgaagtgcaaccctaatctgcattactggaccactcaggacgagggcgccgccatcggcctggcctggattccgtacttcggcccggccgcggaaggcatatacattgagggacttatgcacaatcaggatggactgatttgtggtctgcgccagctggccaacgaaaccacccaggcgctgcagctgttcctccgggcaaccaccgaacttcgcactttctccatcctgaaccggaaggccattgacttcctcttgcaacgctggggaggaacttgccacatcctgggtcctgattgctgcatcgaaccgcatgactggacaaagaacatcaccgacaaaatcgaccagatcatccacgatttcgtggacaagaccctgcctatcgagggccgggggagcggcaagcaaatcgaagataaaatcgaggaaattctgtcaaagatttaccacattgagaacgaaatcgcccggatcaagaagctgataggtcatcaccaccaccatcattgataa

SEQ ID NO: 9 - protein, 633 aa, Artificial Sequence
  Synthetic
  MISC_FEATURE: GP DeltaTM-DeltaFUR-X-HIS protein
MEFWLSWVFLVAILKGVQCVPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVSNGAKNISGQSPARTSSDPGTNTTTEDHKIMASENSSAMVQVHSQGREAAVSHLTTLATISTSPQSLTTKPGPDNSTHNTPVYKLDISEATQVEQHHRRTDNDSTASDTPSATTAAGPPKAENTNTSKSTDFLDPATTTSPQNHSETAGNNNTHHQDTGEESASSGKLGLITNTIAGVAGLITGGAGTAAEAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPIEGRHHHHHH

SEQ ID NO: 10 - DNA, 1905 bp, Artificial Sequence
  Synthetic
  misc_feature: GP DeltaTM-DeltaFUR-X-HIS DNA
atggagttctggttgtcttgggtgtttctcgtcgctattctgaagggggtgcagtgcgtgccactgggagtgatccacaacagcactctccaagtctccgacgtggacaagctcgtgtgccgagacaagctttccagcacaaaccagctgcggtccgtgggactgaacctggagggaaatggcgtggccaccgatgtgccgtctgcaaccaagagatggggcttccgctcgggcgtgcccccgaaagtggtcaactacgaggccggagaatgggccgaaaactgttacaacctggagatcaagaagcccgacggttccgaatgcctccctgccgcccctgacggaatcaggggattcccgagatgccgctacgtgcacaaagtgtccgggactggaccttgtgctggagatttcgccttccacaaggagggcgcctttttcctgtacgaccggctcgcatccaccgtgatctatagaggaaccaccttcgcggaaggagtggtcgccttcctgatcctgccgcaagccaagaaggacttctttagctcccatccactgcgcgagcccgtgaatgccaccgaagatccctcgtccggctattactccacaaccatccggtaccaagccaccgggttcggtaccaacgagactgagtacctgttcgaagtcgacaacctgacttacgtgcagctcgaatcgcggttcacgccacagttcctcctgcaactcaacgaaactatctacactagcgggaaacgctcgaacaccaccgggaagctgatttggaaggtcaaccctgaaatcgacaccaccatcggagagtgggctttttgggagactaagaagaacctcacccggaagatcagatccgaggaattgtccttcactgtggtgtccaacggagcgaagaacatcagcggacagtcccccgcacggacctcatcggacccgggaaccaacaccaccaccgaggaccacaagatcatggcctcggagaactcatccgccatggtgcaagtgcatagccagggccgcgaagccgccgtgtcccacctgactaccctggcgaccatcagcaccagcccgcagtcgctgactactaagcctgggccagacaacagcacccacaacacccctgtgtacaagctggacatttccgaagctactcaggtcgagcagcatcaccggcggaccgacaacgattccaccgcttccgacaccccctccgccaccaccgccgccggcccgccgaaggccgaaaacaccaatacgtcaaagtcgaccgatttccttgaccccgccaccactacgagcccccagaaccactcagaaactgcgggcaacaacaacacccaccaccaggatactggagaggagtccgcatcaagcggaaagctcggcctgattactaacactatcgcgggggtcgctggattgattaccggtggtgccgggaccgcggcagaggccattgtgaacgcgcagccgaagtgcaaccctaatctgcattactggaccactcaggacgagggcgccgccatcggcctggcctggattccgtacttcggcccggccgcggaaggcatatacattgagggacttatgcacaatcaggatggactgatttgtggtctgcgccagctggccaacgaaaccacccaggcgctgcagctgttcctccgggcaaccaccgaacttcgcactttctccatcctgaaccggaaggccattgacttcctcttgcaacgctggggaggaacttgccacatcctgggtcctgattgctgcatcgaaccgcatgactggacaaagaacatcaccgacaaaatcgaccagatcatccacgatttcgtggacaagaccctgcctatcgagggccggcaccatcaccaccaccattagtga

SEQ ID NO: 11 - protein, 483 aa, Artificial Sequence
  Synthetic
  MISC_FEATURE: GP DeltaTM-DeltaMUC-DeltaFUR-X-HIS protein
MEFWLSWVFLVAILKGVQCVPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVNTHHQDTGEESASSGKLGLITNTIAGVAGLITGGAGTAAAEAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPIEGRHHHHHH

SEQ ID NO: 12 - DNA, 1455 bp, Artificial Sequence
  Synthetic
  misc_feature: GP DeltaTM-DeltaMUC-DeltaFUR-X-HIS DNA
atggagttctggttgtcttgggtgtttctcgtcgctattctgaagggggtgcagtgcgtgccactgggagtgatccacaacagcactctccaagtctccgacgtggacaagctcgtgtgccgagacaagctttccagcacaaaccagctgcggtccgtgggactgaacctggagggaaatggcgtggccaccgatgtgccgtctgcaaccaagagatggggcttccgctcgggcgtgcccccgaaagtggtcaactacgaggccggagaatgggccgaaaactgttacaacctggagatcaagaagcccgacggttccgaatgcctccctgccgcccctgacggaatcaggggattcccgagatgccgctacgtgcacaaagtgtccgggactggaccttgtgctggagatttcgccttccacaaggagggcgcctttttcctgtacgaccggctcgcatccaccgtgatctatagaggaaccaccttcgcggaaggagtggtcgccttcctgatcctgccgcaagccaagaaggacttctttagctcccatccactgcgcgagcccgtgaatgccaccgaagatccctcgtccggctattactccacaaccatccggtaccaagccaccgggttcggtaccaacgagactgagtacctgttcgaagtcgacaacctgacttacgtgcagctcgaatcgcggttcacgccacagttcctcctgcaactcaacgaaactatctacactagcgggaaacgctcgaacaccaccgggaagctgatttggaaggtcaaccctgaaatcgacaccaccatcggagagtgggctttttgggagactaagaagaacctcacccggaagatcagatccgaggaattgtccttcaccgtggtcaacacccaccaccaggatactggagaggagtccgcatcaagcggaaagctcggcctgattactaacactatcgcgggggtcgctggattgattaccggtggtgccgggaccgccgcggcagaggccattgtgaacgcgcagccgaagtgcaaccctaatctgcattactggaccactcaggacgagggcgccgccatcggcctggcctggattccgtacttcggcccggccgcggaaggcatatacattgagggacttatgcacaatcaggatggactgatttgtggtctgcgccagctggccaacgaaaccacccaggcgctgcagctgttcctccgggcaaccaccgaacttcgcactttctccatcctgaaccggaaggccattgacttcctcttgcaacgctggggaggaacttgccacatcctgggtcctgattgctgcatcgaaccgcatgactggacaaagaacatcaccgacaaaatcgaccagatcatccacgatttcgtggacaagaccctgcctatcgagggccggcaccatcaccaccaccattagtga

SEQ ID NO: 13 - protein, 623 aa, Artificial Sequence
  Synthetic
  MISC_FEATURE: GP DeltaTM protein
MEFWLSWVFLVAILKGVQCVPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVSNGAKNISGQSPARTSSDPGTNTTTEDHKIMASENSSAMVQVHSQGREAAVSHLTTLATISTSPQSLTTKPGPDNSTHNTPVYKLDISEATQVEQHHRRTDNDSTASDTPSATTAAGPPKAENTNTSKSTDFLDPATTTSPQNHSETAGNNNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLP

SEQ ID NO: 14 - DNA, 1875 bp, Artificial Sequence
  Synthetic
  misc_feature: GP DeltaTM DNA
atggagttctggttgtcttgggtgtttctcgtcgctattctgaagggggtgcagtgcgtgccactgggagtgatccacaacagcactctccaagtctccgacgtggacaagctcgtgtgccgagacaagctttccagcacaaaccagctgcggtccgtgggactgaacctggagggaaatggcgtggccaccgatgtgccgtctgcaaccaagagatggggcttccgctcgggcgtgcccccgaaagtggtcaactacgaggccggagaatgggccgaaaactgttacaacctggagatcaagaagcccgacggttccgaatgcctccctgccgcccctgacggaatcaggggattcccgagatgccgctacgtgcacaaagtgtccgggactggaccttgtgctggagatttcgccttccacaaggagggcgcctttttcctgtacgaccggctcgcatccaccgtgatctatagaggaaccaccttcgcggaaggagtggtcgccttcctgatcctgccgcaagccaagaaggacttctttagctcccatccactgcgcgagcccgtgaatgccaccgaagatccctcgtccggctattactccacaaccatccggtaccaagccaccgggttcggtaccaacgagactgagtacctgttcgaagtcgacaacctgacttacgtgcagctcgaatcgcggttcacgccacagttcctcctgcaactcaacgaaactatctacactagcgggaaacgctcgaacaccaccgggaagctgatttggaaggtcaaccctgaaatcgacaccaccatcggagagtgggctttttgggagactaagaagaacctcacccggaagatcagatccgaggaattgtccttcactgtggtgtccaacggagcgaagaacatcagcggacagtcccccgcacggacctcatcggacccgggaaccaacaccaccaccgaggaccacaagatcatggcctcggagaactcatccgccatggtgcaagtgcatagccagggccgcgaagccgccgtgtcccacctgactaccctggcgaccatcagcaccagcccgcagtcgctgactactaagcctgggccagacaacagcacccacaacacccctgtgtacaagctggacatttccgaagctactcaggtcgagcagcatcaccggcggaccgacaacgattccaccgcttccgacaccccctccgccaccaccgccgccggcccgccgaaggccgaaaacaccaatacgtcaaagtcgaccgatttccttgaccccgccaccactacgagcccccagaaccactcagaaactgcgggcaacaacaacacccaccaccaggatactggagaggagtccgcatcaagcggaaagctcggcctgattactaacactatcgcgggggtcgctggattgattaccggtggtagaaggaccaggagagaggccattgtgaacgcgcagccgaagtgcaaccctaatctgcattactggaccactcaggacgagggcgccgccatcggcctggcctggattccgtacttcggcccggccgcggaaggcatatacattgagggacttatgcacaatcaggatggactgatttgtggtctgcgccagctggccaacgaaaccacccaggcgctgcagctgttcctccgggcaaccaccgaacttcgcactttctccatcctgaaccggaaggccattgacttcctcttgcaacgctggggaggaacttgccacatcctgggtcctgattgctgcatcgaaccgcatgactggacaaagaacatcaccgacaaaatcgaccagatcatccacgatttcgtggacaagaccctgccttagtga

SEQ ID NO: 15 - protein, 472 aa, Artificial Sequence
  Synthetic
  misc_feature: GP_DeltaTM-DeltaMUC protein
MEFWLSWVFLVAILKGVQCVPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLP

SEQ ID NO: 16 - DNA, 1422 bp, Artificial Sequence
  Synthetic
  misc_feature: GP_DeltaTM-DeltaMUC DNA
atggagttctggttgtcttgggtgtttctcgtcgctattctgaagggggtgcagtgcgtgccactgggagtgatccacaacagcactctccaagtctccgacgtggacaagctcgtgtgccgagacaagctttccagcacaaaccagctgcggtccgtgggactgaacctggagggaaatggcgtggccaccgatgtgccgtctgcaaccaagagatggggcttccgctcgggcgtgcccccgaaagtggtcaactacgaggccggagaatgggccgaaaactgttacaacctggagatcaagaagcccgacggttccgaatgcctccctgccgcccctgacggaatcaggggattcccgagatgccgctacgtgcacaaagtgtccgggactggaccttgtgctggagatttcgccttccacaaggagggcgcctttttcctgtacgaccggctcgcatccaccgtgatctatagaggaaccaccttcgcggaaggagtggtcgccttcctgatcctgccgcaagccaagaaggacttctttagctcccatccactgcgcgagcccgtgaatgccaccgaagatccctcgtccggctattactccacaaccatccggtaccaagccaccgggttcggtaccaacgagactgagtacctgttcgaagtcgacaacctgacttacgtgcagctcgaatcgcggttcacgccacagttcctcctgcaactcaacgaaactatctacactagcgggaaacgctcgaacaccaccgggaagctgatttggaaggtcaaccctgaaatcgacaccaccatcggagagtgggctttttgggagactaagaagaacctcacccggaagatcagatccgaggaattgtccttcaccgtggtcaacacccaccaccaggatactggagaggagtccgcatcaagcggaaagctcggcctgattactaacactatcgcgggggtcgctggattgattaccggtggtagaaggaccaggagagaggccattgtgaacgcgcagccgaagtgcaaccctaatctgcattactggaccactcaggacgagggcgccgccatcggcctggcctggattccgtacttcggcccggccgcggaaggcatatacattgagggacttatgcacaatcaggatggactgatttgtggtctgcgccagctggccaacgaaaccacccaggcgctgcagctgttcctccgggcaaccaccgaacttcgcactttctccatcctgaaccggaaggccattgacttcctcttgcaacgctggggaggaacttgccacatcctgggtcctgattgctgcatcgaaccgcatgactggacaaagaacatcaccgacaaaatcgaccagatcatccacgatttcgtggacaagaccctgccttagtga

SEQ ID NO: 17 - protein, 482 aa, Artificial Sequence
  Synthetic
  MISC_FEATURE: GP DeltaTM-CC1-X-HIS
MEFWLSWVFLVAILKGVQCVPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGCVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLCNETTQALQLFLRATTELRTFSILNRKAIDFLLQRWGGTCHILGPDCCIEPHDWTKNITDKIDQIIHDFVDKTLPIEGRHHHHHH

SEQ ID NO: 18 - DNA, 1452 bp, Artificial Sequence
  Synthetic
  misc_feature: GP DeltaTM-CC1-X-HIS
atggagttctggttgtcttgggtgtttctcgtcgctattctgaagggggtgcagtgcgtgccactgggagtgatccacaacagcactctccaagtctccgacgtggacaagctcgtgtgccgagacaagctttccagcacaaaccagctgcggtccgtgggactgaacctggagggaaatggcgtggccaccgatgtgccgtctgcaaccaagagatggggcttccgctcgggcgtgcccccgaaagtggtcaactacgaggccggagaatgggccgaaaactgttacaacctggagatcaagaagcccgacggttccgaatgcctccctgccgcccctgacggaatcaggggattcccgagatgccgctacgtgcacaaagtgtccgggactggaccttgtgctggagatttcgccttccacaaggagggcgcctttttcctgtacgaccggctcgcatccaccgtgatctatagaggaaccaccttcgcggaaggatgcgtcgccttcctgatcctgccgcaagccaagaaggacttctttagctcccatccactgcgcgagcccgtgaatgccaccgaagatccctcgtccggctattactccacaaccatccggtaccaagccaccgggttcggtaccaacgagactgagtacctgttcgaagtcgacaacctgacttacgtgcagctcgaatcgcggttcacgccacagttcctcctgcaactcaacgaaactatctacactagcgggaaacgctcgaacaccaccgggaagctgatttggaaggtcaaccctgaaatcgacaccaccatcggagagtgggctttttgggagactaagaagaacctcacccggaagatcagatccgaggaattgtccttcaccgtggtcaacacccaccaccaggatactggagaggagtccgcatcaagcggaaagctcggcctgattactaacactatcgcgggggtcgctggattgattaccggtggtagaaggaccaggagagaggccattgtgaacgcgcagccgaagtgcaaccctaatctgcattactggaccactcaggacgagggcgccgccatcggcctggcctggattccgtacttcggcccggccgcggaaggcatatacattgagggacttatgcacaatcaggatggactgatttgtggtctgcgccagctgtgcaacgaaaccacccaggcgctgcagctgttcctccgggcaaccaccgaacttcgcactttctccatcctgaaccggaaggccattgacttcctcttgcaacgctggggaggaacttgccacatcctgggtcctgattgctgcatcgaaccgcatgactggacaaagaacatcaccgacaaaatcgaccagatcatccacgatttcgtggacaagaccctgcctatcgagggccggcaccatcaccaccaccattgataa

SEQ ID NO: 19 - protein, 482 aa, Artificial Sequence
  Synthetic
  MISC_FEATURE: GP DeltaTM-CC2-X-HIS protein
MEFWLSWVFLVAILKGVQCVPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFCETKKNLTRKIRSEELSFTVVNTHHQDTGEESASSGKLGLITNTIAGVAGLITGGRRTRREAIVNAQPKCCPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPIEGRHHHHHH

SEQ ID NO: 20 - DNA, 1452 bp, Artificial Sequence
  Synthetic
  misc_feature: GP DeltaTM-CC2-X-HIS DNA
atggagttctggttgtcttgggtgtttctcgtcgctattctgaagggggtgcagtgcgtgccactgggagtgatccacaacagcactctccaagtctccgacgtggacaagctcgtgtgccgagacaagctttccagcacaaaccagctgcggtccgtgggactgaacctggagggaaatggcgtggccaccgatgtgccgtctgcaaccaagagatggggcttccgctcgggcgtgcccccgaaagtggtcaactacgaggccggagaatgggccgaaaactgttacaacctggagatcaagaagcccgacggttccgaatgcctccctgccgcccctgacggaatcaggggattcccgagatgccgctacgtgcacaaagtgtccgggactggaccttgtgctggagatttcgccttccacaaggagggcgcctttttcctgtacgaccggctcgcatccaccgtgatctatagaggaaccaccttcgcggaaggagtggtcgccttcctgatcctgccgcaagccaagaaggacttctttagctcccatccactgcgcgagcccgtgaatgccaccgaagatccctcgtccggctattactccacaaccatccggtaccaagccaccgggttcggtaccaacgagactgagtacctgttcgaagtcgacaacctgacttacgtgcagctcgaatcgcggttcacgccacagttcctcctgcaactcaacgaaactatctacactagcgggaaacgctcgaacaccaccgggaagctgatttggaaggtcaaccctgaaatcgacaccaccatcggagagtgggctttttgcgagactaagaagaacctcacccggaagatcagatccgaggaattgtccttcaccgtggtcaacacccaccaccaggatactggagaggagtccgcatcaagcggaaagctcggcctgattactaacactatcgcgggggtcgctggattgattaccggtggtagaaggaccaggagagaggccattgtgaacgcgcagccgaagtgctgccctaatctgcattactggaccactcaggacgagggcgccgccatcggcctggcctggattccgtacttcggcccggccgcggaaggcatatacattgagggacttatgcacaatcaggatggactgatttgtggtctgcgccagctggccaacgaaaccacccaggcgctgcagctgttcctccgggcaaccaccgaacttcgcactttctccatcctgaaccggaaggccattgacttcctcttgcaacgctggggaggaacttgccacatcctgggtcctgattgctgcatcgaaccgcatgactggacaaagaacatcaccgacaaaatcgaccagatcatccacgatttcgtggacaagaccctgcctatcgagggccggcaccatcaccaccaccattgataa

SEQ ID NO: 21 - protein, 482 aa, Artificial Sequence
  Synthetic
  MISC_FEATURE: GP DeltaTM-CC3-X-HIS protein
MEFWLSWVFLVAILKGVQCVPLGVIHNSTLQVSDVDKLVCRDKLSSTNQLRSVGLNLEGNGVATDVPSATKRWGFRSGVPPKVVNYECGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVNTHHQDTGEESASSGKLGLITNTIAGVAGLITGGRRTRREAIVNAQPKCNPNLHYWCTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPIEGRHHHHHH

SEQ ID NO: 22 - DNA, 1452 bp, Artificial Sequence
  Synthetic
  misc_feature: GP DeltaTM-CC3-X-HIS DNA
atggagttctggttgtcttgggtgtttctcgtcgctattctgaagggggtgcagtgcgtgccactgggagtgatccacaacagcactctccaagtctccgacgtggacaagctcgtgtgccgagacaagctttccagcacaaaccagctgcggtccgtgggactgaacctggagggaaatggcgtggccaccgatgtgccgtctgcaaccaagagatggggcttccgctcgggcgtgcccccgaaagtggtcaactacgagtgcggagaatgggccgaaaactgttacaacctggagatcaagaagcccgacggttccgaatgcctccctgccgcccctgacggaatcaggggattcccgagatgccgctacgtgcacaaagtgtccgggactggaccttgtgctggagatttcgccttccacaaggagggcgcctttttcctgtacgaccggctcgcatccaccgtgatctatagaggaaccaccttcgcggaaggagtggtcgccttcctgatcctgccgcaagccaagaaggacttctttagctcccatccactgcgcgagcccgtgaatgccaccgaagatccctcgtccggctattactccacaaccatccggtaccaagccaccgggttcggtaccaacgagactgagtacctgttcgaagtcgacaacctgacttacgtgcagctcgaatcgcggttcacgccacagttcctcctgcaactcaacgaaactatctacactagcgggaaacgctcgaacaccaccgggaagctgatttggaaggtcaaccctgaaatcgacaccaccatcggagagtgggctttttgggagactaagaagaacctcacccggaagatcagatccgaggaattgtccttcaccgtggtcaacacccaccaccaggatactggagaggagtccgcatcaagcggaaagctcggcctgattactaacactatcgcgggggtcgctggattgattaccggtggtagaaggaccaggagagaggccattgtgaacgcgcagccgaagtgcaaccctaatctgcattactggtgcactcaggacgagggcgccgccatcggcctggcctggattccgtacttcggcccggccgcggaaggcatatacattgagggacttatgcacaatcaggatggactgatttgtggtctgcgccagctggccaacgaaaccacccaggcgctgcagctgttcctccgggcaaccaccgaacttcgcactttctccatcctgaaccggaaggccattgacttcctcttgcaacgctggggaggaacttgccacatcctgggtcctgattgctgcatcgaaccgcatgactggacaaagaacatcaccgacaaaatcgaccagatcatccacgatttcgtggacaagaccctgcctatcgagggccggcaccatcaccaccaccattgataa

SEQ ID NO: 23 - protein, 482 aa, Artificial Sequence
  Synthetic
  MISC_FEATURE: GP DeltaTM-CC4-X-HIS protein
MEFWLSWVFLVAILKGVQCVPLGVIHNSTLQVSDVDKLVCRDKLSSTNQLRSVGLNLEGNGVATDVPSATKRWGFRSGVPPKVVNYECGEWAENCYNLEIKKPDGSECLPAAPDGIRGFPRCRYVHKVSGTGPCAGDFAFHKEGAFFLYDRLASTVIYRGTTFAEGVVAFLILPQAKKDFCSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPIEGRHHHHHH

SEQ ID NO: 24 - DNA, 1452 bp, Artificial Sequence
  Synthetic
  misc_feature: GP DeltaTM-CC4-X-HIS DNA
atggagttctggttgtcttgggtgtttctcgtcgctattctgaagggggtgcagtgcgtgccactgggagtgatccacaacagcactctccaagtctccgacgtggacaagctcgtgtgccgagacaagctttccagcacaaaccagctgcggtccgtgggactgaacctggagggaaatggcgtggccaccgatgtgccgtctgcaaccaagagatggggcttccgctcgggcgtgcccccgaaagtggtcaactacgagtgcggagaatgggccgaaaactgttacaacctggagatcaagaagcccgacggttccgaatgcctccctgccgcccctgacggaatcaggggattcccgagatgccgctacgtgcacaaagtgtccgggactggaccttgtgctggagatttcgccttccacaaggagggcgcctttttcctgtacgaccggctcgcatccaccgtgatctatagaggaaccaccttcgcggaaggagtggtcgccttcctgatcctgccgcaagccaagaaggacttctgcagctcccatccactgcgcgagcccgtgaatgccaccgaagatccctcgtccggctattactccacaaccatccggtaccaagccaccgggttcggtaccaacgagactgagtacctgttcgaagtcgacaacctgacttacgtgcagctcgaatcgcggttcacgccacagttcctcctgcaactcaacgaaactatctacactagcgggaaacgctcgaacaccaccgggaagctgatttggaaggtcaaccctgaaatcgacaccaccatcggagagtgggctttttgggagactaagaagaacctcacccggaagatcagatccgaggaattgtccttcaccgtggtcaacacccaccaccaggatactggagaggagtccgcatcaagcggaaagctcggcctgattactaacactatcgcgggggtcgctggattgattaccggtggtagaaggaccaggagagaggccattgtgaacgcgcagccgaagtgcaaccctaatctgcattactggaccactcaggacgagggcgccgccatcggcctggcctggattccgtacttcggcccggccgcggaaggcatatacattgagggacttatgcacaatcaggatggactgatttgtggtctgcgccagctggccaacgaaaccacccaggcgctgcagctgttcctccgggcaaccaccgaacttcgcactttctccatcctgaaccggaaggccattgacttcctcttgcaacgctggggaggaacttgccacatcctgggtcctgattgctgcatcgaaccgcatgactggacaaagaacatcaccgacaaaatcgaccagatcatccacgatttcgtggacaagaccctgcctatcgagggccggcaccatcaccaccaccattgataa

SEQ ID NO: 25 - protein, 482 aa, Artificial Sequence
  Synthetic
  MISC_FEATURE: GP DeltaTM-CC5-X-HIS protein
MEFWLSWVFLVAILKGVQCVPLGVIHNSTLQVSDVDKLVCRDKLSSTNQLRSVGLNLEGNGVATDVPSATKRWGFRSGVPPKVCNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRCFSILNRKAIDFLLQRWGGTCHILGPDCCIEPHDWTKNITDKIDQIIHDFVDKTLPIEGRHHHHHH

SEQ ID NO: 26 - DNA, 1452 bp, Artificial Sequence
  Synthetic
  misc_feature: GP DeltaTM-CC5-X-HIS DNA
atggagttctggttgtcttgggtgtttctcgtcgctattctgaagggggtgcagtgcgtgccactgggagtgatccacaacagcactctccaagtctccgacgtggacaagctcgtgtgccgagacaagctttccagcacaaaccagctgcggtccgtgggactgaacctggagggaaatggcgtggccaccgatgtgccgtctgcaaccaagagatggggcttccgctcgggcgtgcccccgaaagtgtgtaactacgaggccggagaatgggccgaaaactgttacaacctggagatcaagaagcccgacggttccgaatgcctccctgccgcccctgacggaatcaggggattcccgagatgccgctacgtgcacaaagtgtccgggactggaccttgtgctggagatttcgccttccacaaggagggcgcctttttcctgtacgaccggctcgcatccaccgtgatctatagaggaaccaccttcgcggaaggagtggtcgccttcctgatcctgccgcaagccaagaaggacttctttagctcccatccactgcgcgagcccgtgaatgccaccgaagatccctcgtccggctattactccacaaccatccggtaccaagccaccgggttcggtaccaacgagactgagtacctgttcgaagtcgacaacctgacttacgtgcagctcgaatcgcggttcacgccacagttcctcctgcaactcaacgaaactatctacactagcgggaaacgctcgaacaccaccgggaagctgatttggaaggtcaaccctgaaatcgacaccaccatcggagagtgggctttttgggagactaagaagaacctcacccggaagatcagatccgaggaattgtccttcaccgtggtcaacacccaccaccaggatactggagaggagtccgcatcaagcggaaagctcggcctgattactaacactatcgcgggggtcgctggattgattaccggtggtagaaggaccaggagagaggccattgtgaacgcgcagccgaagtgcaaccctaatctgcattactggaccactcaggacgagggcgccgccatcggcctggcctggattccgtacttcggcccggccgcggaaggcatatacattgagggacttatgcacaatcaggatggactgatttgtggtctgcgccagctggccaacgaaaccacccaggcgctgcagctgttcctccgggcaaccaccgaacttcgctgtttctccatcctgaaccggaaggccattgacttcctcttgcaacgctggggaggaacttgccacatcctgggtcctgattgctgcatcgaaccgcatgactggacaaagaacatcaccgacaaaatcgaccagatcatccacgatttcgtggacaagaccctgcctatcgagggccggcaccatcaccaccaccattgataa

SEQ ID NO: 27 - protein, 482 aa, Artificial Sequence
  Synthetic
  MISC_FEATURE: GP DeltaTM-CC6-X-HIS protein
MEFWLSWVFLVAILKGVQCVPLGVIHNSTLQVSDVDKLVCRDKLSSTNQCRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSICNRKAIDFLLQRWGGTCHILGPDCCIEPHDWTKNITDKIDQIIHDFVDKTLPIEGRHHHHHH

SEQ ID NO: 28 - DNA, 1452 bp, Artificial Sequence
  Synthetic
  misc_feature: GP DeltaTM-CC6-X-HIS DNA
atggagttctggttgtcttgggtgtttctcgtcgctattctgaagggggtgcagtgcgtgccactgggagtgatccacaacagcactctccaagtctccgacgtggacaagctcgtgtgccgagacaagctttccagcacaaaccagtgccggtccgtgggactgaacctggagggaaatggcgtggccaccgatgtgccgtctgcaaccaagagatggggcttccgctcgggcgtgcccccgaaagtggtcaactacgaggccggagaatgggccgaaaactgttacaacctggagatcaagaagcccgacggttccgaatgcctccctgccgcccctgacggaatcaggggattcccgagatgccgctacgtgcacaaagtgtccgggactggaccttgtgctggagatttcgccttccacaaggagggcgcctttttcctgtacgaccggctcgcatccaccgtgatctatagaggaaccaccttcgcggaaggagtggtcgccttcctgatcctgccgcaagccaagaaggacttctttagctcccatccactgcgcgagcccgtgaatgccaccgaagatccctcgtccggctattactccacaaccatccggtaccaagccaccgggttcggtaccaacgagactgagtacctgttcgaagtcgacaacctgacttacgtgcagctcgaatcgcggttcacgccacagttcctcctgcaactcaacgaaactatctacactagcgggaaacgctcgaacaccaccgggaagctgatttggaaggtcaaccctgaaatcgacaccaccatcggagagtgggctttttgggagactaagaagaacctcacccggaagatcagatccgaggaattgtccttcaccgtggtcaacacccaccaccaggatactggagaggagtccgcatcaagcggaaagctcggcctgattactaacactatcgcgggggtcgctggattgattaccggtggtagaaggaccaggagagaggccattgtgaacgcgcagccgaagtgcaaccctaatctgcattactggaccactcaggacgagggcgccgccatcggcctggcctggattccgtacttcggcccggccgcggaaggcatatacattgagggacttatgcacaatcaggatggactgatttgtggtctgcgccagctggccaacgaaaccacccaggcgctgcagctgttcctccgggcaaccaccgaacttcgcactttctccatctgcaaccggaaggccattgacttcctcttgcaacgctggggaggaacttgccacatcctgggtcctgattgctgcatcgaaccgcatgactggacaaagaacatcaccgacaaaatcgaccagatcatccacgatttcgtggacaagaccctgcctatcgagggccggcaccatcaccaccaccattgataa

SEQ ID NO: 29 - protein, 482 aa, Artificial Sequence
  Synthetic
  MISC_FEATURE: GP DeltaTM-CC7-X-HIS protein
MEFWLSWVFLVAILKGVQCVPLGVIHNCTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVNTHHQDTGEESASSGKLGLITNTIAGVAGLITGGRRTRREAIVNAQPKCNPNLHYWTTQDEGAAIGLAWIPYFGPAAEGIYIEGLMHNQCGLICGLRQLANETTQALQLFLRATTELRTFSILNRKAIDFLLQRWGGTCHILGPDCCIEPHDWTKNITDKIDQIIHDFVDKTLPIEGRHHHHHH

SEQ ID NO: 30 - DNA, 1452 bp, Artificial Sequence
  Synthetic
  misc_feature: GP DeltaTM-CC7-X-HIS DNA
atggagttctggttgtcttgggtgtttctcgtcgctattctgaagggggtgcagtgcgtgccactgggagtgatccacaactgcactctccaagtctccgacgtggacaagctcgtgtgccgagacaagctttccagcacaaaccagctgcggtccgtgggactgaacctggagggaaatggcgtggccaccgatgtgccgtctgcaaccaagagatggggcttccgctcgggcgtgcccccgaaagtggtcaactacgaggccggagaatgggccgaaaactgttacaacctggagatcaagaagcccgacggttccgaatgcctccctgccgcccctgacggaatcaggggattcccgagatgccgctacgtgcacaaagtgtccgggactggaccttgtgctggagatttcgccttccacaaggagggcgcctttttcctgtacgaccggctcgcatccaccgtgatctatagaggaaccaccttcgcggaaggagtggtcgccttcctgatcctgccgcaagccaagaaggacttctttagctcccatccactgcgcgagcccgtgaatgccaccgaagatccctcgtccggctattactccacaaccatccggtaccaagccaccgggttcggtaccaacgagactgagtacctgttcgaagtcgacaacctgacttacgtgcagctcgaatcgcggttcacgccacagttcctcctgcaactcaacgaaactatctacactagcgggaaacgctcgaacaccaccgggaagctgatttggaaggtcaaccctgaaatcgacaccaccatcggagagtgggctttttgggagactaagaagaacctcacccggaagatcagatccgaggaattgtccttcaccgtggtcaacacccaccaccaggatactggagaggagtccgcatcaagcggaaagctcggcctgattactaacactatcgcgggggtcgctggattgattaccggtggtagaaggaccaggagagaggccattgtgaacgcgcagccgaagtgcaaccctaatctgcattactggaccactcaggacgagggcgccgccatcggcctggcctggattccgtacttcggcccggccgcggaaggcatatacattgagggacttatgcacaatcagtgcggactgatttgtggtctgcgccagctggccaacgaaaccacccaggcgctgcagctgttcctccgggcaaccaccgaacttcgcactttctccatcctgaaccggaaggccattgacttcctcttgcaacgctggggaggaacttgccacatcctgggtcctgattgctgcatcgaaccgcatgactggacaaagaacatcaccgacaaaatcgaccagatcatccacgatttcgtggacaagaccctgcctatcgagggccggcaccatcaccaccaccattgataa

SEQ ID NO: 31 - protein, 482 aa, Artificial Sequence
  Synthetic
  MISC_FEATURE: GP DeltaTM-CC8-X-HIS protein
MEFWLSWVFLVAILKGVQCVPLGVIHNSTLQVSDVDKLVCRDKLSSTNQLRSVGLNLEGNGVATDVPSATKRWGFRSGVPPKVCNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFCETKKNLTRKIRSEELSFTVVNTHHQDTGEESASSGKLGLITNTIAGVAGLITGGRRTRREAIVNAQPKCCPNLHYWTTQDEGAAIGLAWIPYFGPAAEGIYIEGLMHNQDGLICGLRQLANETTQALQLFLRATTELRCFSILNRKAIDFLLQRWGGTCHILGPDCCIEPHDWTKNITDKIDQIIHDFVDKTLPIEGRHHHHHH

SEQ ID NO: 32 - DNA, 1452 bp, Artificial Sequence
  Synthetic
  misc_feature: GP DeltaTM-CC8-X-HIS DNA
atggagttctggttgtcttgggtgtttctcgtcgctattctgaagggggtgcagtgcgtgccactgggagtgatccacaacagcactctccaagtctccgacgtggacaagctcgtgtgccgagacaagctttccagcacaaaccagctgcggtccgtgggactgaacctggagggaaatggcgtggccaccgatgtgccgtctgcaaccaagagatggggcttccgctcgggcgtgcccccgaaagtgtgcaactacgaggccggagaatgggccgaaaactgttacaacctggagatcaagaagcccgacggttccgaatgcctccctgccgcccctgacggaatcaggggattcccgagatgccgctacgtgcacaaagtgtccgggactggaccttgtgctggagatttcgccttccacaaggagggcgcctttttcctgtacgaccggctcgcatccaccgtgatctatagaggaaccaccttcgcggaaggagtggtcgccttcctgatcctgccgcaagccaagaaggacttctttagctcccatccactgcgcgagcccgtgaatgccaccgaagatccctcgtccggctattactccacaaccatccggtaccaagccaccgggttcggtaccaacgagactgagtacctgttcgaagtcgacaacctgacttacgtgcagctcgaatcgcggttcacgccacagttcctcctgcaactcaacgaaactatctacactagcgggaaacgctcgaacaccaccgggaagctgatttggaaggtcaaccctgaaatcgacaccaccatcggagagtgggctttttgcgagactaagaagaacctcacccggaagatcagatccgaggaattgtccttcaccgtggtcaacacccaccaccaggatactggagaggagtccgcatcaagcggaaagctcggcctgattactaacactatcgcgggggtcgctggattgattaccggtggtagaaggaccaggagagaggccattgtgaacgcgcagccgaagtgctgccctaatctgcattactggaccactcaggacgagggcgccgccatcggcctggcctggattccgtacttcggcccggccgcggaaggcatatacattgagggacttatgcacaatcaggatggactgatttgtggtctgcgccagctggccaacgaaaccacccaggcgctgcagctgttcctccgggcaaccaccgaacttcgctgtttctccatcctgaaccggaaggccattgacttcctcttgcaacgctggggaggaacttgccacatcctgggtcctgattgctgcatcgaaccgcatgactggacaaagaacatcaccgacaaaatcgaccagatcatccacgatttcgtggacaagaccctgcctatcgagggccggcaccatcaccaccaccattgataa

SEQ ID NO: 33 - protein, 482 aa, Artificial Sequence
  Synthetic
  MISC_FEATURE: GP DeltaTM-CC9-X-HIS protein
MEFWLSWVFLVAILKGVQCVPLGVIHNCTLQVSDVDKLVCRDKLSSTNQLRSVGLNLEGNGVATDVPSATKRWGFRSGVPPKVVNYECGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVNTHHQDTGEESASSGKLGLITNTIAGVAGLITGGRRTRREAIVNAQPKCNPNLHYWCTQDEGAAIGLAWIPYFGPAAEGIYIEGLMHNQCGLICGLRQLANETTQALQLFLRATTELRTFSILNRKAIDFLLQRWGGTCHILGPDCCIEPHDWTKNITDKIDQIIHDFVDKTLPIEGRHHHHHH

SEQ ID NO: 34 - DNA, 1452 bp, Artificial Sequence
  Synthetic
  misc_feature: GP DeltaTM-CC9-X-HIS DNA
atggagttctggttgtcttgggtgtttctcgtcgctattctgaagggggtgcagtgcgtgccactgggagtgatccacaactgcactctccaagtctccgacgtggacaagctcgtgtgccgagacaagctttccagcacaaaccagctgcggtccgtgggactgaacctggagggaaatggcgtggccaccgatgtgccgtctgcaaccaagagatggggcttccgctcgggcgtgcccccgaaagtggtcaactacgagtgcggagaatgggccgaaaactgttacaacctggagatcaagaagcccgacggttccgaatgcctccctgccgcccctgacggaatcaggggattcccgagatgccgctacgtgcacaaagtgtccgggactggaccttgtgctggagatttcgccttccacaaggagggcgcctttttcctgtacgaccggctcgcatccaccgtgatctatagaggaaccaccttcgcggaaggagtggtcgccttcctgatcctgccgcaagccaagaaggacttctttagctcccatccactgcgcgagcccgtgaatgccaccgaagatccctcgtccggctattactccacaaccatccggtaccaagccaccgggttcggtaccaacgagactgagtacctgttcgaagtcgacaacctgacttacgtgcagctcgaatcgcggttcacgccacagttcctcctgcaactcaacgaaactatctacactagcgggaaacgctcgaacaccaccgggaagctgatttggaaggtcaaccctgaaatcgacaccaccatcggagagtgggctttttgggagactaagaagaacctcacccggaagatcagatccgaggaattgtccttcaccgtggtcaacacccaccaccaggatactggagaggagtccgcatcaagcggaaagctcggcctgattactaacactatcgcgggggtcgctggattgattaccggtggtagaaggaccaggagagaggccattgtgaacgcgcagccgaagtgcaaccctaatctgcattactggtgtactcaggacgagggcgccgccatcggcctggcctggattccgtacttcggcccggccgcggaaggcatatacattgagggacttatgcacaatcagtgtggactgatttgtggtctgcgccagctggccaacgaaaccacccaggcgctgcagctgttcctccgggcaaccaccgaacttcgcactttctccatcctgaaccggaaggccattgacttcctcttgcaacgctggggaggaacttgccacatcctgggtcctgattgctgcatcgaaccgcatgactggacaaagaacatcaccgacaaaatcgaccagatcatccacgatttcgtggacaagaccctgcctatcgagggccggcaccatcaccaccaccattgataa

SEQ ID NO: 35 - protein, 482 aa, Artificial Sequence
  Synthetic
  MISC_FEATURE: GP DeltaTM-CC10-X-HIS protein
MEFWLSWVFLVAILKGVQCVPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLCYVQLESRFTPQFLLQLNETIYTSGKRCNTTGKLIWKVNPEIDTTIGEWAFWETKKNLTRKIRSEELSFTVVNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPIEGRHHHHHH

SEQ ID NO: 36 - DNA, 1452 bp, Artificial Sequence
  Synthetic
  misc_feature: GP DeltaTM-CC10-X-HIS DNA
atggagttctggttgtcttgggtgtttctcgtcgctattctgaagggggtgcagtgcgtgccactgggagtgatccacaacagcactctccaagtctccgacgtggacaagctcgtgtgccgagacaagctttccagcacaaaccagctgcggtccgtgggactgaacctggagggaaatggcgtggccaccgatgtgccgtctgcaaccaagagatggggcttccgctcgggcgtgcccccgaaagtggtcaactacgaggccggagaatgggccgaaaactgttacaacctggagatcaagaagcccgacggttccgaatgcctccctgccgcccctgacggaatcaggggattcccgagatgccgctacgtgcacaaagtgtccgggactggaccttgtgctggagatttcgccttccacaaggagggcgcctttttcctgtacgaccggctcgcatccaccgtgatctatagaggaaccaccttcgcggaaggagtggtcgccttcctgatcctgccgcaagccaagaaggacttctttagctcccatccactgcgcgagcccgtgaatgccaccgaagatccctcgtccggctattactccacaaccatccggtaccaagccaccgggttcggtaccaacgagactgagtacctgttcgaagtcgacaacctgtgctacgtgcagctcgaatcgcggttcacgccacagttcctcctgcaactcaacgaaactatctacactagcgggaaacgctgcaacaccaccgggaagctgatttggaaggtcaaccctgaaatcgacaccaccatcggagagtgggctttttgggagactaagaagaacctcacccggaagatcagatccgaggaattgtccttcaccgtggtcaacacccaccaccaggatactggagaggagtccgcatcaagcggaaagctcggcctgattactaacactatcgcgggggtcgctggattgattaccggtggtagaaggaccaggagagaggccattgtgaacgcgcagccgaagtgcaaccctaatctgcattactggaccactcaggacgagggcgccgccatcggcctggcctggattccgtacttcggcccggccgcggaaggcatatacattgagggacttatgcacaatcaggatggactgatttgtggtctgcgccagctggccaacgaaaccacccaggcgctgcagctgttcctccgggcaaccaccgaacttcgcactttctccatcctgaaccggaaggccattgacttcctcttgcaacgctggggaggaacttgccacatcctgggtcctgattgctgcatcgaaccgcatgactggacaaagaacatcaccgacaaaatcgaccagatcatccacgatttcgtggacaagaccctgcctatcgagggccggcaccatcaccaccaccattgataa

SEQ ID NO: 37 - protein, 513 aa, Artificial Sequence
  Synthetic
  MISC_FEATURE: GP DeltaTM-DeltaMUC-T4-X-HIS protein
MEFWLSWVFLVAILKGVQCVPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPGSGYIPEAPRDGQAYVRKDGEWVLLSTFLGTIEGRHHHHHH

SEQ ID NO: 38 - DNA, 1545 bp, Artificial Sequence
  Synthetic
  misc_feature: GP DeltaTM-DeltaMUC-T4-X-HIS DNA
atggagttctggttgtcttgggtgtttctcgtcgctattctgaagggggtgcagtgcgtgccactgggagtgatccacaacagcactctccaagtctccgacgtggacaagctcgtgtgccgagacaagctttccagcacaaaccagctgcggtccgtgggactgaacctggagggaaatggcgtggccaccgatgtgccgtctgcaaccaagagatggggcttccgctcgggcgtgcccccgaaagtggtcaactacgaggccggagaatgggccgaaaactgttacaacctggagatcaagaagcccgacggttccgaatgcctccctgccgcccctgacggaatcaggggattcccgagatgccgctacgtgcacaaagtgtccgggactggaccttgtgctggagatttcgccttccacaaggagggcgcctttttcctgtacgaccggctcgcatccaccgtgatctatagaggaaccaccttcgcggaaggagtggtcgccttcctgatcctgccgcaagccaagaaggacttctttagctcccatccactgcgcgagcccgtgaatgccaccgaagatccctcgtccggctattactccacaaccatccggtaccaagccaccgggttcggtaccaacgagactgagtacctgttcgaagtcgacaacctgacttacgtgcagctcgaatcgcggttcacgccacagttcctcctgcaactcaacgaaactatctacactagcgggaaacgctcgaacaccaccgggaagctgatttggaaggtcaaccctgaaatcgacaccaccatcggagagtgggctttttgggagactaagaagaacctcacccggaagatcagatccgaggaattgtccttcaccgtggtcaacacccaccaccaggatactggagaggagtccgcatcaagcggaaagctcggcctgattactaacactatcgcgggggtcgctggattgattaccggtggtagaaggaccaggagagaggccattgtgaacgcgcagccgaagtgcaaccctaatctgcattactggaccactcaggacgagggcgccgccatcggcctggcctggattccgtacttcggcccggccgcggaaggcatatacattgagggacttatgcacaatcaggatggactgatttgtggtctgcgccagctggccaacgaaaccacccaggcgctgcagctgttcctccgggcaaccaccgaacttcgcactttctccatcctgaaccggaaggccattgacttcctcttgcaacgctggggaggaacttgccacatcctgggtcctgattgctgcatcgaaccgcatgactggacaaagaacatcaccgacaaaatcgaccagatcatccacgatttcgtggacaagaccctgcctggatcaggttacatccccgaggccccgagagacggccaggcctacgtgcggaaggacggcgaatgggtcctcctgtccaccttccttgggactatcgaagggcgccaccatcaccaccaccattgataa

SEQ ID NO: 39 - protein, 503 aa, Artificial Sequence
  Synthetic
  MISC_FEATURE: GP DeltaTM-DeltaMUC-T4 protein
MEFWLSWVFLVAILKGVQCVPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPGSGYIPEAPRDGQAYVRKDGEWVLLSTFLGT

SEQ ID NO: 40 - DNA, 1515 bp, Artificial Sequence
  Synthetic
  misc_feature: GP DeltaTM-DeltaMUC-T4 DNA
atggagttctggttgtcttgggtgtttctcgtcgctattctgaagggggtgcagtgcgtgccactgggagtgatccacaacagcactctccaagtctccgacgtggacaagctcgtgtgccgagacaagctttccagcacaaaccagctgcggtccgtgggactgaacctggagggaaatggcgtggccaccgatgtgccgtctgcaaccaagagatggggcttccgctcgggcgtgcccccgaaagtggtcaactacgaggccggagaatgggccgaaaactgttacaacctggagatcaagaagcccgacggttccgaatgcctccctgccgcccctgacggaatcaggggattcccgagatgccgctacgtgcacaaagtgtccgggactggaccttgtgctggagatttcgccttccacaaggagggcgcctttttcctgtacgaccggctcgcatccaccgtgatctatagaggaaccaccttcgcggaaggagtggtcgccttcctgatcctgccgcaagccaagaaggacttctttagctcccatccactgcgcgagcccgtgaatgccaccgaagatccctcgtccggctattactccacaaccatccggtaccaagccaccgggttcggtaccaacgagactgagtacctgttcgaagtcgacaacctgacttacgtgcagctcgaatcgcggttcacgccacagttcctcctgcaactcaacgaaactatctacactagcgggaaacgctcgaacaccaccgggaagctgatttggaaggtcaaccctgaaatcgacaccaccatcggagagtgggctttttgggagactaagaagaacctcacccggaagatcagatccgaggaattgtccttcaccgtggtcaacacccaccaccaggatactggagaggagtccgcatcaagcggaaagctcggcctgattactaacactatcgcgggggtcgctggattgattaccggtggtagaaggaccaggagagaggccattgtgaacgcgcagccgaagtgcaaccctaatctgcattactggaccactcaggacgagggcgccgccatcggcctggcctggattccgtacttcggcccggccgcggaaggcatatacattgagggacttatgcacaatcaggatggactgatttgtggtctgcgccagctggccaacgaaaccacccaggcgctgcagctgttcctccgggcaaccaccgaacttcgcactttctccatcctgaaccggaaggccattgacttcctcttgcaacgctggggaggaacttgccacatcctgggtcctgattgctgcatcgaaccgcatgactggacaaagaacatcaccgacaaaatcgaccagatcatccacgatttcgtggacaagaccctgcctggatcaggttacatccccgaggccccgagagacggccaggcctacgtgcggaaggacggcgaatgggtcctcctgtccaccttccttgggacttgataa

SEQ ID NO: 41 - protein, 514 aa, Artificial Sequence
  Synthetic
  MISC_FEATURE: GP DeltaTM-DeltaMUC-GCN4-X-HIS protein
MEFWLSWVFLVAILKGVQCVPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPGSGKQIEDKIEEILSKIYHIENEIARIKKLIGIEGRHHHHHH

SEQ ID NO: 42 - DNA, 1548 bp, Artificial Sequence
  Synthetic
  misc_feature: GP DeltaTM-DeltaMUC-GCN4-X-HIS DNA
atggagttctggttgtcttgggtgtttctcgtcgctattctgaagggggtgcagtgcgtgccactgggagtgatccacaacagcactctccaagtctccgacgtggacaagctcgtgtgccgagacaagctttccagcacaaaccagctgcggtccgtgggactgaacctggagggaaatggcgtggccaccgatgtgccgtctgcaaccaagagatggggcttccgctcgggcgtgcccccgaaagtggtcaactacgaggccggagaatgggccgaaaactgttacaacctggagatcaagaagcccgacggttccgaatgcctccctgccgcccctgacggaatcaggggattcccgagatgccgctacgtgcacaaagtgtccgggactggaccttgtgctggagatttcgccttccacaaggagggcgcctttttcctgtacgaccggctcgcatccaccgtgatctatagaggaaccaccttcgcggaaggagtggtcgccttcctgatcctgccgcaagccaagaaggacttctttagctcccatccactgcgcgagcccgtgaatgccaccgaagatccctcgtccggctattactccacaaccatccggtaccaagccaccgggttcggtaccaacgagactgagtacctgttcgaagtcgacaacctgacttacgtgcagctcgaatcgcggttcacgccacagttcctcctgcaactcaacgaaactatctacactagcgggaaacgctcgaacaccaccgggaagctgatttggaaggtcaaccctgaaatcgacaccaccatcggagagtgggctttttgggagactaagaagaacctcacccggaagatcagatccgaggaattgtccttcaccgtggtcaacacccaccaccaggatactggagaggagtccgcatcaagcggaaagctcggcctgattactaacactatcgcgggggtcgctggattgattaccggtggtagaaggaccaggagagaggccattgtgaacgcgcagccgaagtgcaaccctaatctgcattactggaccactcaggacgagggcgccgccatcggcctggcctggattccgtacttcggcccggccgcggaaggcatatacattgagggacttatgcacaatcaggatggactgatttgtggtctgcgccagctggccaacgaaaccacccaggcgctgcagctgttcctccgggcaaccaccgaacttcgcactttctccatcctgaaccggaaggccattgacttcctcttgcaacgctggggaggaacttgccacatcctgggtcctgattgctgcatcgaaccgcatgactggacaaagaacatcaccgacaaaatcgaccagatcatccacgatttcgtggacaagaccctgcctgggagcggcaagcaaatcgaagataaaatcgaggaaattctgtcaaagatttaccacattgagaacgaaatcgcccggatcaagaagctgataggtatcgagggccggcatcaccaccaccatcattgataa

SEQ ID NO: 43 - protein, 504 aa, Artificial Sequence
  Synthetic
  MISC_FEATURE: GP DeltaTM-DeltaMUC-GCN4 protein
MEFWLSWVFLVAILKGVQCVPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPGSGKQIEDKIEEILSKIYHIENEIARIKKLIG

SEQ ID NO: 44 - DNA, 1518 bp, Artificial Sequence
  Synthetic
  misc_feature: GP DeltaTM-DeltaMUC-GCN4 DNA
atggagttctggttgtcttgggtgtttctcgtcgctattctgaagggggtgcagtgcgtgccactgggagtgatccacaacagcactctccaagtctccgacgtggacaagctcgtgtgccgagacaagctttccagcacaaaccagctgcggtccgtgggactgaacctggagggaaatggcgtggccaccgatgtgccgtctgcaaccaagagatggggcttccgctcgggcgtgcccccgaaagtggtcaactacgaggccggagaatgggccgaaaactgttacaacctggagatcaagaagcccgacggttccgaatgcctccctgccgcccctgacggaatcaggggattcccgagatgccgctacgtgcacaaagtgtccgggactggaccttgtgctggagatttcgccttccacaaggagggcgcctttttcctgtacgaccggctcgcatccaccgtgatctatagaggaaccaccttcgcggaaggagtggtcgccttcctgatcctgccgcaagccaagaaggacttctttagctcccatccactgcgcgagcccgtgaatgccaccgaagatccctcgtccggctattactccacaaccatccggtaccaagccaccgggttcggtaccaacgagactgagtacctgttcgaagtcgacaacctgacttacgtgcagctcgaatcgcggttcacgccacagttcctcctgcaactcaacgaaactatctacactagcgggaaacgctcgaacaccaccgggaagctgatttggaaggtcaaccctgaaatcgacaccaccatcggagagtgggctttttgggagactaagaagaacctcacccggaagatcagatccgaggaattgtccttcaccgtggtcaacacccaccaccaggatactggagaggagtccgcatcaagcggaaagctcggcctgattactaacactatcgcgggggtcgctggattgattaccggtggtagaaggaccaggagagaggccattgtgaacgcgcagccgaagtgcaaccctaatctgcattactggaccactcaggacgagggcgccgccatcggcctggcctggattccgtacttcggcccggccgcggaaggcatatacattgagggacttatgcacaatcaggatggactgatttgtggtctgcgccagctggccaacgaaaccacccaggcgctgcagctgttcctccgggcaaccaccgaacttcgcactttctccatcctgaaccggaaggccattgacttcctcttgcaacgctggggaggaacttgccacatcctgggtcctgattgctgcatcgaaccgcatgactggacaaagaacatcaccgacaaaatcgaccagatcatccacgatttcgtggacaagaccctgcctgggagcggcaagcaaatcgaagataaaatcgaggaaattctgtcaaagatttaccacattgagaacgaaatcgcccggatcaagaagctgataggttgataa

SEQ ID NO: 45 - protein, 664 aa, Artificial Sequence
  Synthetic
  MISC_FEATURE: GP DeltaTM-T4-X-HIS protein
MEFWLSWVFLVAILKGVQCVPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVSNGAKNISGQSPARTSSDPGTNTTTEDHKIMASENSSAMVQVHSQGREAAVSHLTTLATISTSPQSLTTKPGPDNSTHNTPVYKLDISEATQVEQHHRRTDNDSTASDTPSATTAAGPPKAENTNTSKSTDFLDPATTTSPQNHSETAGNNNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPGSGYIPEAPRDGQAYVRKDGEWVLLSTFLGTIEGRHHHHHH

SEQ ID NO: 46 - DNA, 1998 bp, Artificial Sequence
  Synthetic
  misc_feature: GP DeltaTM-T4-X-HIS DNA
atggagttctggttgtcttgggtgtttctcgtcgctattctgaagggggtgcagtgcgtgccactgggagtgatccacaacagcactctccaagtctccgacgtggacaagctcgtgtgccgagacaagctttccagcacaaaccagctgcggtccgtgggactgaacctggagggaaatggcgtggccaccgatgtgccgtctgcaaccaagagatggggcttccgctcgggcgtgcccccgaaagtggtcaactacgaggccggagaatgggccgaaaactgttacaacctggagatcaagaagcccgacggttccgaatgcctccctgccgcccctgacggaatcaggggattcccgagatgccgctacgtgcacaaagtgtccgggactggaccttgtgctggagatttcgccttccacaaggagggcgcctttttcctgtacgaccggctcgcatccaccgtgatctatagaggaaccaccttcgcggaaggagtggtcgccttcctgatcctgccgcaagccaagaaggacttctttagctcccatccactgcgcgagcccgtgaatgccaccgaagatccctcgtccggctattactccacaaccatccggtaccaagccaccgggttcggtaccaacgagactgagtacctgttcgaagtcgacaacctgacttacgtgcagctcgaatcgcggttcacgccacagttcctcctgcaactcaacgaaactatctacactagcgggaaacgctcgaacaccaccgggaagctgatttggaaggtcaaccctgaaatcgacaccaccatcggagagtgggctttttgggagactaagaagaacctcacccggaagatcagatccgaggaattgtccttcactgtggtgtccaacggagcgaagaacatcagcggacagtcccccgcacggacctcatcggacccgggaaccaacaccaccaccgaggaccacaagatcatggcctcggagaactcatccgccatggtgcaagtgcatagccagggccgcgaagccgccgtgtcccacctgactaccctggcgaccatcagcaccagcccgcagtcgctgactactaagcctgggccagacaacagcacccacaacacccctgtgtacaagctggacatttccgaagctactcaggtcgagcagcatcaccggcggaccgacaacgattccaccgcttccgacaccccctccgccaccaccgccgccggcccgccgaaggccgaaaacaccaatacgtcaaagtcgaccgatttccttgaccccgccaccactacgagcccccagaaccactcagaaactgcgggcaacaacaacacccaccaccaggatactggagaggagtccgcatcaagcggaaagctcggcctgattactaacactatcgcgggggtcgctggattgattaccggtggtagaaggaccaggagagaggccattgtgaacgcgcagccgaagtgcaaccctaatctgcattactggaccactcaggacgagggcgccgccatcggcctggcctggattccgtacttcggcccggccgcggaaggcatatacattgagggacttatgcacaatcaggatggactgatttgtggtctgcgccagctggccaacgaaaccacccaggcgctgcagctgttcctccgggcaaccaccgaacttcgcactttctccatcctgaaccggaaggccattgacttcctcttgcaacgctggggaggaacttgccacatcctgggtcctgattgctgcatcgaaccgcatgactggacaaagaacatcaccgacaaaatcgaccagatcatccacgatttcgtggacaagaccctgcctggatcaggttacatccccgaggccccgagagacggccaggcctacgtgcggaaggacggcgaatgggtcctcctgtccaccttccttgggactatcgaagggcgccaccatcaccaccaccattgataa

SEQ ID NO: 47 - protein, 654 aa, Artificial Sequence
  Synthetic
  MISC_FEATURE: GP DeltaTM-T4 protein
MEFWLSWVFLVAILKGVQCVPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVSNGAKNISGQSPARTSSDPGTNTTTEDHKIMASENSSAMVQVHSQGREAAVSHLTTLATISTSPQSLTTKPGPDNSTHNTPVYKLDISEATQVEQHHRRTDNDSTASDTPSATTAAGPPKAENTNTSKSTDFLDPATTTSPQNHSETAGNNNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPGSGYIPEAPRDGQAYVRKDGEWVLLSTFLGT

SEQ ID NO: 48 - DNA, 1968 bp, Artificial Sequence
  Synthetic
  misc_feature: GP DeltaTM-T4 DNA
atggagttctggttgtcttgggtgtttctcgtcgctattctgaagggggtgcagtgcgtgccactgggagtgatccacaacagcactctccaagtctccgacgtggacaagctcgtgtgccgagacaagctttccagcacaaaccagctgcggtccgtgggactgaacctggagggaaatggcgtggccaccgatgtgccgtctgcaaccaagagatggggcttccgctcgggcgtgcccccgaaagtggtcaactacgaggccggagaatgggccgaaaactgttacaacctggagatcaagaagcccgacggttccgaatgcctccctgccgcccctgacggaatcaggggattcccgagatgccgctacgtgcacaaagtgtccgggactggaccttgtgctggagatttcgccttccacaaggagggcgcctttttcctgtacgaccggctcgcatccaccgtgatctatagaggaaccaccttcgcggaaggagtggtcgccttcctgatcctgccgcaagccaagaaggacttctttagctcccatccactgcgcgagcccgtgaatgccaccgaagatccctcgtccggctattactccacaaccatccggtaccaagccaccgggttcggtaccaacgagactgagtacctgttcgaagtcgacaacctgacttacgtgcagctcgaatcgcggttcacgccacagttcctcctgcaactcaacgaaactatctacactagcgggaaacgctcgaacaccaccgggaagctgatttggaaggtcaaccctgaaatcgacaccaccatcggagagtgggctttttgggagactaagaagaacctcacccggaagatcagatccgaggaattgtccttcactgtggtgtccaacggagcgaagaacatcagcggacagtcccccgcacggacctcatcggacccgggaaccaacaccaccaccgaggaccacaagatcatggcctcggagaactcatccgccatggtgcaagtgcatagccagggccgcgaagccgccgtgtcccacctgactaccctggcgaccatcagcaccagcccgcagtcgctgactactaagcctgggccagacaacagcacccacaacacccctgtgtacaagctggacatttccgaagctactcaggtcgagcagcatcaccggcggaccgacaacgattccaccgcttccgacaccccctccgccaccaccgccgccggcccgccgaaggccgaaaacaccaatacgtcaaagtcgaccgatttccttgaccccgccaccactacgagcccccagaaccactcagaaactgcgggcaacaacaacacccaccaccaggatactggagaggagtccgcatcaagcggaaagctcggcctgattactaacactatcgcgggggtcgctggattgattaccggtggtagaaggaccaggagagaggccattgtgaacgcgcagccgaagtgcaaccctaatctgcattactggaccactcaggacgagggcgccgccatcggcctggcctggattccgtacttcggcccggccgcggaaggcatatacattgagggacttatgcacaatcaggatggactgatttgtggtctgcgccagctggccaacgaaaccacccaggcgctgcagctgttcctccgggcaaccaccgaacttcgcactttctccatcctgaaccggaaggccattgacttcctcttgcaacgctggggaggaacttgccacatcctgggtcctgattgctgcatcgaaccgcatgactggacaaagaacatcaccgacaaaatcgaccagatcatccacgatttcgtggacaagaccctgcctggatcaggttacatccccgaggccccgagagacggccaggcctacgtgcggaaggacggcgaatgggtcctcctgtccaccttccttgggacttgataa

SEQ ID NO: 49 - protein, 665 aa, Artificial Sequence
  Synthetic
  MISC_FEATURE: GP DeltaTM-GCN4-X-HIS protein
MEFWLSWVFLVAILKGVQCVPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVSNGAKNISGQSPARTSSDPGTNTTTEDHKIMASENSSAMVQVHSQGREAAVSHLTTLATISTSPQSLTTKPGPDNSTHNTPVYKLDISEATQVEQHHRRTDNDSTASDTPSATTAAGPPKAENTNTSKSTDFLDPATTTSPQNHSETAGNNNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPGSGKQIEDKIEEILSKIYHIENEIARIKKLIGIEGRHHHHHH

SEQ ID NO: 50 - DNA, 2001 bp, Artificial Sequence
  Synthetic
  misc_feature: GP DeltaTM-GCN4-X-HIS DNA
atggagttctggttgtcttgggtgtttctcgtcgctattctgaagggggtgcagtgcgtgccactgggagtgatccacaacagcactctccaagtctccgacgtggacaagctcgtgtgccgagacaagctttccagcacaaaccagctgcggtccgtgggactgaacctggagggaaatggcgtggccaccgatgtgccgtctgcaaccaagagatggggcttccgctcgggcgtgcccccgaaagtggtcaactacgaggccggagaatgggccgaaaactgttacaacctggagatcaagaagcccgacggttccgaatgcctccctgccgcccctgacggaatcaggggattcccgagatgccgctacgtgcacaaagtgtccgggactggaccttgtgctggagatttcgccttccacaaggagggcgcctttttcctgtacgaccggctcgcatccaccgtgatctatagaggaaccaccttcgcggaaggagtggtcgccttcctgatcctgccgcaagccaagaaggacttctttagctcccatccactgcgcgagcccgtgaatgccaccgaagatccctcgtccggctattactccacaaccatccggtaccaagccaccgggttcggtaccaacgagactgagtacctgttcgaagtcgacaacctgacttacgtgcagctcgaatcgcggttcacgccacagttcctcctgcaactcaacgaaactatctacactagcgggaaacgctcgaacaccaccgggaagctgatttggaaggtcaaccctgaaatcgacaccaccatcggagagtgggctttttgggagactaagaagaacctcacccggaagatcagatccgaggaattgtccttcactgtggtgtccaacggagcgaagaacatcagcggacagtcccccgcacggacctcatcggacccgggaaccaacaccaccaccgaggaccacaagatcatggcctcggagaactcatccgccatggtgcaagtgcatagccagggccgcgaagccgccgtgtcccacctgactaccctggcgaccatcagcaccagcccgcagtcgctgactactaagcctgggccagacaacagcacccacaacacccctgtgtacaagctggacatttccgaagctactcaggtcgagcagcatcaccggcggaccgacaacgattccaccgcttccgacaccccctccgccaccaccgccgccggcccgccgaaggccgaaaacaccaatacgtcaaagtcgaccgatttccttgaccccgccaccactacgagcccccagaaccactcagaaactgcgggcaacaacaacacccaccaccaggatactggagaggagtccgcatcaagcggaaagctcggcctgattactaacactatcgcgggggtcgctggattgattaccggtggtagaaggaccaggagagaggccattgtgaacgcgcagccgaagtgcaaccctaatctgcattactggaccactcaggacgagggcgccgccatcggcctggcctggattccgtacttcggcccggccgcggaaggcatatacattgagggacttatgcacaatcaggatggactgatttgtggtctgcgccagctggccaacgaaaccacccaggcgctgcagctgttcctccgggcaaccaccgaacttcgcactttctccatcctgaaccggaaggccattgacttcctcttgcaacgctggggaggaacttgccacatcctgggtcctgattgctgcatcgaaccgcatgactggacaaagaacatcaccgacaaaatcgaccagatcatccacgatttcgtggacaagaccctgcctgggagcggcaagcaaatcgaagataaaatcgaggaaattctgtcaaagatttaccacattgagaacgaaatcgcccggatcaagaagctgataggtatcgagggccggcatcaccaccaccatcattgataa

SEQ ID NO: 51 - protein, 655 aa, Artificial Sequence
  Synthetic
  MISC_FEATURE: GP DeltaTM-GCN4 protein
MEFWLSWVFLVAILKGVQCVPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVSNGAKNISGQSPARTSSDPGTNTTTEDHKIMASENSSAMVQVHSQGREAAVSHLTTLATISTSPQSLTTKPGPDNSTHNTPVYKLDISEATQVEQHHRRTDNDSTASDTPSATTAAGPPKAENTNTSKSTDFLDPATTTSPQNHSETAGNNNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPGSGKQIEDKIEEILSKIYHIENEIARIKKLIG

SEQ ID NO: 52 - DNA, 1971 bp, Artificial Sequence
  Synthetic
  misc_feature: GP DeltaTM-GCN4 DNA
atggagttctggttgtcttgggtgtttctcgtcgctattctgaagggggtgcagtgcgtgccactgggagtgatccacaacagcactctccaagtctccgacgtggacaagctcgtgtgccgagacaagctttccagcacaaaccagctgcggtccgtgggactgaacctggagggaaatggcgtggccaccgatgtgccgtctgcaaccaagagatggggcttccgctcgggcgtgcccccgaaagtggtcaactacgaggccggagaatgggccgaaaactgttacaacctggagatcaagaagcccgacggttccgaatgcctccctgccgcccctgacggaatcaggggattcccgagatgccgctacgtgcacaaagtgtccgggactggaccttgtgctggagatttcgccttccacaaggagggcgcctttttcctgtacgaccggctcgcatccaccgtgatctatagaggaaccaccttcgcggaaggagtggtcgccttcctgatcctgccgcaagccaagaaggacttctttagctcccatccactgcgcgagcccgtgaatgccaccgaagatccctcgtccggctattactccacaaccatccggtaccaagccaccgggttcggtaccaacgagactgagtacctgttcgaagtcgacaacctgacttacgtgcagctcgaatcgcggttcacgccacagttcctcctgcaactcaacgaaactatctacactagcgggaaacgctcgaacaccaccgggaagctgatttggaaggtcaaccctgaaatcgacaccaccatcggagagtgggctttttgggagactaagaagaacctcacccggaagatcagatccgaggaattgtccttcactgtggtgtccaacggagcgaagaacatcagcggacagtcccccgcacggacctcatcggacccgggaaccaacaccaccaccgaggaccacaagatcatggcctcggagaactcatccgccatggtgcaagtgcatagccagggccgcgaagccgccgtgtcccacctgactaccctggcgaccatcagcaccagcccgcagtcgctgactactaagcctgggccagacaacagcacccacaacacccctgtgtacaagctggacatttccgaagctactcaggtcgagcagcatcaccggcggaccgacaacgattccaccgcttccgacaccccctccgccaccaccgccgccggcccgccgaaggccgaaaacaccaatacgtcaaagtcgaccgatttccttgaccccgccaccactacgagcccccagaaccactcagaaactgcgggcaacaacaacacccaccaccaggatactggagaggagtccgcatcaagcggaaagctcggcctgattactaacactatcgcgggggtcgctggattgattaccggtggtagaaggaccaggagagaggccattgtgaacgcgcagccgaagtgcaaccctaatctgcattactggaccactcaggacgagggcgccgccatcggcctggcctggattccgtacttcggcccggccgcggaaggcatatacattgagggacttatgcacaatcaggatggactgatttgtggtctgcgccagctggccaacgaaaccacccaggcgctgcagctgttcctccgggcaaccaccgaacttcgcactttctccatcctgaaccggaaggccattgacttcctcttgcaacgctggggaggaacttgccacatcctgggtcctgattgctgcatcgaaccgcatgactggacaaagaacatcaccgacaaaatcgaccagatcatccacgatttcgtggacaagaccctgcctgggagcggcaagcaaatcgaagataaaatcgaggaaattctgtcaaagatttaccacattgagaacgaaatcgcccggatcaagaagctgataggttgataa

SEQ ID NO: 53 - protein, 151 aa, Artificial Sequence
  Synthetic
  MISC_FEATURE: Mucin-like domain Sequence
SNGAKNISGQSPARTSSDPGTNTTTEDHKIMASENSSAMVQVHSQGREAAVSHLTTLATISTSPQSLTTKPGPDNSTHNTPVYKLDISEATQVEQHHRRTDNDSTASDTPSATTAAGPPKAENTNTSKSTDFLDPATTTSPQNHSETAGNN

SEQ ID NO: 54 - protein, 31 aa, Artificial Sequence
  Synthetic
  MISC_FEATURE: T4 trimerization sequence
GSGYIPEAPRDGQAYVRKDGEWVLLSTFLGT

SEQ ID NO: 55 - protein, 32 aa, Artificial Sequence
  Synthetic
  MISC_FEATURE: GCN4 trimerization sequence
GSGKQIEDKIEEILSKIYHIENEIARIKKLIG

SEQ ID NO: 56 - protein, 27 aa, Artificial Sequence
  Synthetic
  MISC_FEATURE: Transmembrane domain sequence
QWIPAGIGVTGVIIAVIALFCICKFVF

(19) United States
(12) Patent Application Publication
(10) Pub. No.: US 2021/0340185 A1
(43) Pub. Date: Nov. 4, 2021

(54) EBOLA VACCINE COMPOSITIONS AND METHODS OF USING SAME

(71) Applicants: CENTRE HOSPITALIER UNIVERSITAIRE VAUDOIS, Lausanne (CH); EXCELLGENE SA, Monthey (CH)

(72) Inventors: Divor Kiseljak, Renens (CH); Florian M. Wurm, Montreux (CH); Valentina Agnolon, Lausanne (CH); Francois Spertini, Lausanne (CH); Bruno Correia, St-Saphorin-Sur-Morges (CH)

(21) Appl. No.: 17/271,657

(22) PCT Filed: Aug. 28, 2019
(86) PCT No.: PCT/IB2019/057260

§ 371 Date: Feb. 26, 2021

Related U.S. Application Data
(60) Provisional application No. 62/724,242, filed on Aug. 29, 2018.

(51) Int. Cl. C07K 14/005; A61K 39/12; A61P 31/14; A61P 37/04

(52) U.S. Cl. CPC C07K 14/005; A61K 39/12; A61P 31/14; A61K 2039/54; C07K 2319/21; C07K 2319/50; A61P 37/04

(57) ABSTRACT

Materials and methods for the prevention of Ebola virus are provided. This disclosure is related to vaccine compositions comprising one or more Ebola vims (EBOV) glycoproteins as well as methods of preventing an EBOV infection comprising administering such compositions. In particular, modified EBOV glycoproteins are provided that form trimers and induce an immune response.

This application contains, as a separate part of the disclosure, a Sequence Listing in computer-readable form which is incorporated by reference in its entirety and identified as follows: Filename:53435ASeqlisting.txt; Size: 185,269 bytes: Created: Aug. 21, 2019.

### FIELD OF THE INVENTION

This invention is related to vaccine compositions comprising one or more Ebola virus (EBOV) glycoproteins as well as methods of preventing an EBOV infection comprising administering such compositions.

### BACKGROUND

The Ebola virus (EBOV) is a member of Filoviridae family, which is highly contagious for human and non-human primates, and causes severe hemorrhagic fever associated with 50-90% human mortality [Lee, J E., et al. “Structure of the Ebola virus glycoprotein bound to an antibody from a human survivor.” Nature 454.7201 (2008): 177.]. Currently, there is no prophylactic or therapeutic EBOV approved for human usage. Conventional vaccine development approaches based on virus inactivation were shown to be ineffective [Marzi, A, and H Feldmann. “Ebola virus vaccines: an overview of current approaches.” Expert review of vaccines 13.4 (2014): 521-531]. Among the 13 vaccine candidates in various development phases, the majority are based on recombinant viral vectors such as Vesicular Stomatitis Virus (VSV), Modified Vaccinia Ankara virus (MVA), and human or chimpanzee Adenovirus (Ad) [Ohimain, E I. “Recent advances in the development of vaccines for Ebola virus disease.” Virus research 211 (2016): 174-185.]. Recombinant viral vectors have been identified as promising for inducing an anti-EBPV immune response due to their ability to induce potent insert-specific cellular immunity and high levels of antibodies [Venkatraman, N, et al. “Vaccines against Ebola virus.” Vaccine (2017).]. However, these vector-based vaccines against EBOV have been primarily developed in the course of an epidemic or to fight bioterrorism, and they present major limitations for human vaccination, including: I) induction and/or pre-existence of anti-vector neutralizing antibodies, which commonly limit the efficacy of the immune response induced by viral vaccines [McCoy, K, et al. “Effect of preexisting immunity to adenovirus human serotype 5 antigens on the immune responses of nonhuman primates to vaccine regimens based on human- or chimpanzee-derived adenovirus vectors.” Journal of virology 81.12 (2007): 6594-6604.]; II) restricted use of replicating vaccine platforms like rVSV in target populations where immunosuppressive conditions (e.g. HIV, TB, malaria) are common; Ill) need for a strict cold chain storage at −80° C. to ensure vaccine stability and biological activity making these vaccines unsuitable for large-scale vaccination in sub-Saharan countries.

The majority of current vaccine candidates target the surface EBOV glycoprotein (GP), which is the only protein expressed on the virus surface and plays a critical role in EBOV infection [Lee, J E, et al. “Structure of the Ebola virus glycoprotein bound to an antibody from a human survivor.” Nature 454.7201 (2008): 177.]. Humoral responses of EBOV outbreaks survivors mainly target the GP protein, and anti-GP neutralizing antibodies have been associated with protection against EBOV infection [Rimoin, A W, et al. “Ebola Virus Neutralizing Antibodies Detectable in Survivors of the Yambuku, Zaire Outbreak 40 Years after Infection.” The Journal of infectious diseases 217.2 (2017): 223-231. Flyak, Andrew I., et al. “Cross-reactive and potent neutralizing antibody responses in human survivors of natural ebolavirus infection.” Cell 164.3 (2016): 392-405. Dye, J M, et al. “Postexposure antibody prophylaxis protects nonhuman primates from filovirus disease.” Proceedings of the National Academy of Sciences 109.13 (2012): 5034-5039. Qiu, X, et al. “Reversion of advanced Ebola virus disease in nonhuman primates with ZMapp.” Nature 514.7520 (2014): 47. Marzi, A, et al. “Antibodies are necessary for rVSV/ZEBOV-GP-mediated protection against lethal Ebola virus challenge in nonhuman primates.” Proceedings of the National Academy of Sciences 110.5 (2013): 1893-1898.]. Moreover, pre-clinical animal studies for Ebola GP vaccine efficacy have shown the induction of strong T-cell and humoral responses, as well as protection from in vitro/in vivo challenge [Stanley, D A., et al. “Chimpanzee adenovirus vaccine generates acute and durable protective immunity against ebolavirus challenge.” Nature medicine 20.10 (2014): 1126.].

A panel of human neutralizing antibodies directed against Ebola GP has been isolated from donors that recovered from EBOV infection, among which KZ52 [Maruyama, T, et al. “Recombinant human monoclonal antibodies to Ebola virus.” The Journal of infectious diseases179.Supplement_1 (1999): S235-S239.], mAb100, and mAb114 [Corti, D, et al. “Protective monotherapy against lethal Ebola virus infection by a potently neutralizing antibody.” Science (2016): aad5224. Misasi, J, et al. “Structural and molecular basis for Ebola virus neutralization by protective human antibodies.” Science 351.6279 (2016): 1343-1346.]. Moreover, the passive transfer of purified IgG from survivors or the treatment with neutralizing mAb cocktails have been demonstrated to provide protection from viral infection [Dye, J M., et al. “Postexposure antibody prophylaxis protects nonhuman primates from filovirus disease.” Proceedings of the National Academy of Sciences 109.13 (2012): 5034-5039. Qiu, X, et al. “Reversion of advanced Ebola virus disease in nonhuman primates with ZMapp.” Nature 514.7520 (2014): 47. Marzi, A, et al. “Antibodies are necessary for rVSV/ZEBOV-GP-mediated protection against lethal Ebola virus challenge in nonhuman primates.” Proceedings of the National Academy of Sciences 110.5 (2013): 1893-1898.]. Current post-exposure therapies are based on ZMapp, an optimized combination of three mAbs that was shown to rescue 100% of lethally infected nonhuman primates [Pettitt, J, et al. “Therapeutic intervention of Ebola virus infection in rhesus macaques with the MB-003 monoclonal antibody cocktail.” Science translational medicine 5.199 (2013): 199ra113-199ra113; Qiu, X, et al. “Sustained protection against Ebola virus infection following treatment of infected nonhuman primates with ZMAb.” Scientific reports 3 (2013): 3365. Qiu, X, et al. “Reversion of advanced Ebola virus disease in nonhuman primates with ZMapp.” Nature 514.7520 (2014): 47; Qiu, X, et al. “Two-mAb cocktail protects macaques against the Makona variant of Ebola virus.” Science translational medicine 8.329 (2016): 329ra33-329ra33.]. The epitopes recognized by each cocktail component have been recently mapped over the GP protein [Murin, C D., et al. “Structures of protective antibodies reveal sites of vulnerability on Ebola virus.” Proceedings of the National Academy of Sciences 111.48 (2014): 17182-17187.]. The majority of these mAbs show neutralizing activity when administered as a cocktail [Pettitt, J, et al. “Therapeutic intervention of Ebola virus infection in rhesus macaques with the MB-003 monoclonal antibody cocktail.” Science translational medicine 5.199 (2013): 199ra113-199ra113.], highlighting the importance of targeting multiple GP epitopes for complete efficacy. Despite its proved efficacy, the ZMapp treatment has raised concerns in terms of susceptibility to re-infection of the treated survivors [Qiu, X, et al. “Reversion of advanced Ebola virus disease in nonhuman primates with ZMapp.” Nature 514.7520 (2014): 47.].

### SUMMARY

Materials and methods for preventing EBOV are provided herein. In one embodiment, an isolated polypeptide antigen is provided comprising an Ebola virus glycoprotein (EBOV GP) comprising one or more modifications selected from the group consisting of (a) transmembrane and intracellular tail sequence deletion; (b) mucin region deletion; (c) T4 domain insertion; (d) GCN4 domain insertion; (e) a Factor Xa protease recognition sequence; and (f) a histidine tag sequence. In another embodiment, an isolated polypeptide antigen is provided comprising an EBOV GP comprising a transmembrane and intracellular tail sequence deletion, a mucin region deletion, and a T4 domain insertion. In still another embodiment, an isolated polypeptide antigen comprising an EBOV GP comprising a transmembrane and intracellular tail sequence deletion, a mucin region deletion, and a GCN4 domain insertion.

In related embodiments, an aforementioned polypeptide is provided that is capable of eliciting an immunogenic response. In other related embodiments, an aforementioned polypeptide is provided that is capable of being bound by antibodies known to bind wild-type EBOV GP.

In one embodiment of the present disclosure, an isolated polypeptide antigen is provided comprising or consisting of an amino acid sequence that is at least 80% identical to a sequence as set out in any one or more of SEQ ID NOs: 1, 3, 5, 7, 9, 11, 13, 15, 17, 19, 21, 23, 25, 27, 29, 31, 33, 35, 37, 39, 41, 43, 45, 47, 49 or 51, or a fragment, analog or derivative thereof, wherein said polypeptide, fragment, analog or derivative is capable of eliciting an immune response specific to the polypeptide antigen. In a related embodiment, the polypeptide comprises or consists of an amino acid sequence that is 100% identical to a sequence as set out in any one or more of SEQ ID NOs: 1, 3, 5, 7, 9, 11, 13, 15, 17, 19, 21, 23, 25, 27, 29, 31, 33, 35, 37, 39, 41, 43, 45, 47, 49 or 51.

In another embodiment, an aforementioned polypeptide comprises SEQ ID NO: 5, 7, 39 or 43. In one embodiment, the polypeptide comprises SEQ ID NO: 43.

In another embodiment, an aforementioned polypeptide is provided wherein said polypeptide is cleaved into two subunits that are linked by a disulfide bond, thereby forming a heterodimer. In still another embodiment, the heterodimer assembles with two additional heterodimers comprising the aforementioned polypeptides, thereby forming a trimeric conformation.

In another embodiment of the disclosure, a polynucleotide is provided comprising a nucleotide sequence encoding an aforementioned polypeptide. In another embodiment, a vector comprising the polynucleotide is provided. In still another embodiment, an expression vector comprising the polynucleotide operably linked to an expression control sequence is provided.

Another embodiment of the disclosure provides a recombinant host cell comprising the aforementioned vector or the aforementioned expression vector. In another embodiment, the recombinant host cell is (i) a eukaryotic cell selected from the group consisting of mammalian, yeast, insect, plant, amphibian and avian cells; or (ii) a prokaryotic cell. In one embodiment, the host cell is a Chinese Hamster Ovary (CHO) cell.

In still another embodiment, an antigenic composition is provided comprising an aforementioned polypeptide, wherein the polypeptide is present in the composition at a concentration of about 0.1-2000 μg/ml, in a pharmaceutically acceptable carrier, diluent, stabilizer, preservative, or adjuvant.

In still another embodiment, a method of producing an immune response to a Ebola virus in a subject is provided, comprising administering to the subject an effective amount of an aforementioned antigenic composition, thereby producing an immune response to a Ebola virus in the subject. In still another embodiment, a method of preventing a disease or disorder caused by an Ebola virus infection in a subject is provided, comprising administering to the subject an effective amount of an aforementioned composition, thereby preventing a disease or disorder caused by an Ebola virus infection in the subject. In yet another embodiment, a method of immunizing a mammalian subject against an Ebola virus infection is provided comprising administering to the subject an effective amount of an aforementioned antigenic composition, thereby immunizing the subject against an Ebola virus infection. In related embodiments, an aforementioned method if provided wherein the administering is intramuscular administration.

In still other embodiments of the present disclosure, a method of producing an aforementioned polypeptide is provided comprising introducing into a host cell an aforementioned vector under conditions such that the cell produces the polypeptide. In one related embodiment, the host cell is a CHO cell.

Reference throughout this specification to “one embodiment”, “some embodiments” or “an embodiment” means that a particular feature, structure or characteristic described in connection with the embodiment is included in at least one embodiment of the present disclosure. The particular features, structures, or characteristics described herein may be combined in any suitable manner, and all such combinations are contemplated as aspects of the invention.

Unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

The headings herein are for the convenience of the reader and not intended to be limiting. Additional aspects, embodiments, and variations of the invention will be apparent from the Detailed Description and/or Drawing and/or claims.

Although the Applicant invented the full scope of the invention described herein, the Applicant does not intend to claim subject matter described in the prior art work of others. Therefore, in the event that statutory prior art within the scope of a claim is brought to the attention of the Applicant by a Patent Office or other entity or individual, the Applicant reserves the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### BRIEF DESCRIPTION OF THE DRAWINGS

FIG. 1 shows a plasmid Map of the pXLG 6 vector for the expression of DNA of interest (ITR indicates the piggyBac terminal repeat sequences, Puro-R: Resistance marker for puromycin). The DNA of interest for expression is inserted down-stream of the EF-1-alpha intron element, driven by a Cytomegalovirus promoter followed by an Elongation Factor 1 alpha Intron A.

FIG. 2 shows a plasmid Map of pXLG5: “Mobilizing” expression vector in co-transfections. Helper synthetic cDNA indicates the position of the transposase gene, driven by the human cytomegalovirus immediate early promoter.

FIG. 3: Diagram of the Histidine tag-free Ebola GP1/2 variant protein, named “GP ΔTM-ΔMUC-T4”, designed for eventual GMP production in stable, clonally derived CHO cells. The optimized expression cassette contains the DNA sequence derived from a human IgG1 encoding for the Leader Peptide, a CHO codon optimized GP1 sequence under deletion of the sequence encoding the mucin region and encoding a part of the GP2 protein, under deletion of the transmembrane and intracellular sequence, replaced by the T4 trimerization domain. The gap in the diagram indicates a site where, within cells furin, a cellular protease, will cleave the protein into two separate peptide segments. The S—S labeled lines indicate positions where, through intramolecular disulfide bridges the individual GP1/2 peptides are linked and keep the GP1 and GP2 sections of the GP ΔTM-ΔMUC-T4 protein as a monomer together. The short dark box at the carboxy-terminus of the structure indicates a short “T4” trimerization peptide. The * indicates the fact that the GP1 and GP2 sequences do not represent the Ebola wildtype protein sequences.

FIG. 4: Various GP constructs were screened in a direct ELISA for antigenicity. Proteins were immobilized (0.6 μg/ml) on an amino-binding ELISA plate, in order to minimize protein denaturation induced by normal adsorption to the plastic. FIG. 4A: Detection with mAb KZ52 or the serum from an Ebola survivor (HUG). FIG. 4B. and FIG. 4C: Detection with mAbs provided by a collaborating laboratory at the Commissariat a'l Energie Atomique et aux Energies Alternatives (CEA) in France. Mean values are shown.

FIG. 5: Various GP constructs were screened in sandwich ELISAs to identify the most promising ones. The chimeric rabbit KZ52 mAb was used as coating antibody (2 ug/ml) and was immobilized on a Nunc Maxisorp plate. FIG. 5A. Detection with mAb KZ52 or the serum from an Ebola survivor (HUG). FIG. 5B and FIG. 5C: Detection with mAbs provided by a collaborating laboratory at the Commissariat a'l Energie Atomique et aux Energies Alternatives (CEA) in France. Mean values are shown.

FIG. 6: CD secondary structure and thermal stability profiles of GP ΔTM-X-HIS, GP ΔTM-ΔMUC-X-HIS, GP ΔTM-ΔMUC-T4-X-HIS, and GP ΔTM-ΔMUC-GCN4-X-HIS. Spectra were registered between 190 and 250 nm and between 20° C. and 90° C. at 5 degrees intervals. For clarity, only spectra at 20, 75, and 90° C. are shown.

FIG. 7: SPR evaluation of affinities among GP ΔTM-X-HIS, GP ΔTM-ΔMUC-X-HIS, GP ΔTM-ΔMUC-T4-X-HIS, or GP ΔTM-ΔMUC-GCN4-X-HIS and Fab114. Dotted lines represent the actual measured curves while continuous lines represent the fitting. Respective KD values are reported within each panel of the figure.

FIG. 8: Sandwich ELISA analysis of GP ΔTM-X-HIS, GP ΔTM-ΔMUC-X-HIS, GP ΔTM-ΔMUC-T4-X-HIS, and GP ΔTM-ΔMUC-GCN4-X-HIS. The human survivor-derived rabbit KZ52 chimeric monoclonal antibody was used as coating antibody (2 μg/ml) and was immobilized on a Nunc Maxisorp plate to favor its adsorption. The top graph shows the results of the sandwich ELISA with human antibodies—KZ52 or HUG—as detection reagents. The bottom graph shows the results of the sandwich ELISA with a panel of mouse mAbs provided by a collaborating laboratory at the Commissariat a'l Energie Atomique et aux Energies Alternatives (CEA) in France as detection reagents. Average blank values were subtracted from sample values. Column heights represent the mean values of 3 assays, with corresponding standard deviations.

FIG. 9: Evaluation of the inhibitory activity of GP ΔTM-X-HIS, GP ΔTM-ΔMUC-X-HIS, GP ΔTM-ΔMUC-T4-X-HIS, and GP ΔTM-ΔMUC-GCN4-X-HIS on a pseudo-type infection assay in presence of EZP01S, EZP16S, or EZP35S as neutralizing antibodies (assay performed by Dr. L. Bellanger, French Alternative Energies and Atomic Energy Commission, CEA, France). When possible, dilution points were interpolated with a four-parameter dose-response curve.

FIG. 10: A panel of 10 sera obtained from clinical trial volunteers (indicated as Nx, Lx, Tx, Mx) were incubated with pseudo-viruses expressing a Δmuc version of the Ebola GP protein (assay performed by Dr. L. Bellanger, CEA). Volunteers were previously screened in a direct ELISA for preferential recognition of the native GP protein (N) or of the GP protein whether native or denatured (L, i.e. recognition of linear epitopes present both in native and denatured proteins). In addition, some individuals of the represented panel had sera mainly directed against the GP ΔTM-X-HIS protein (T, both when native or denatured) or the GP ΔTM-ΔMUC-X-HIS (M, both when native or when denatured). The different affinities of the sera from volunteers for the pseudo-virus-GP are reflected in the different % of infected cells. Dilution points were interpolated with a four-parameter dose-response curve, for extrapolation of the EC50 values, which are reported in the figure. Sera recognizing the native GP protein have the highest affinity values, assumed to result in an increased protection from infection.

FIG. 11: A panel of 10 sera derived from clinical trial volunteers (left graph) and a panel of 10 Ebola virus survivors (right graph) were analyzed in direct ELISAs to test the recognition of GP ΔTM-X-HIS, GP ΔTM-ΔMUC-X-HIS, GP ΔTM-ΔMUC-T4-X-HIS, and GP ΔTM-ΔMUC-GCN4-X-HIS. Proteins were immobilized (0.6 μg/ml) on an amino-binding ELISA plate, in order to minimize protein denaturation induced by the adsorption to the plastic. Median values are shown together with interquartile ranges. Differences among groups were analyzed with a Kruskal-Wallis one-way ANOVA for multiple comparisons.

FIG. 12: A panel of 10 sera obtained from clinical trial volunteers (round symbols) and a panel of 10 Ebola virus survivors (square symbols) were analyzed in a competition ELISA to test their respective affinities for the monomer GP ΔTM-ΔMUC-X-HIS or for the GP ΔTM-ΔMUC-T4-X-HIS or -GCN4-X-HIS trimers. Median values with IQR values are represented. Statistical significance among groups was evaluated by means of the Wilcoxon test (among the same group of individuals) and the Mann-Whitney test (comparison of volunteers vs survivors).

FIG. 13: A panel of 10 sera obtained from clinical trial volunteers (upper graph) and a panel of 10 Ebola virus survivors (lower graph) were analyzed in direct ELISA to test the respective amount of IgG1 (black circles) and IgG2 (white circles) subclasses. Median with IQR values are represented.

FIG. 14: A panel of 10 sera derived from clinical trial volunteers (black circles) and a panel of 10 Ebola virus survivors (white circles) were analyzed in direct ELISA to test the respective amount of IgM antibodies. Median values with IQR values are represented.

FIG. 15: T-cell Elispot performed on sera from volunteers of the Lausanne clinical trial, whose T-cells were stimulated with a pool of 15-mers overlapping peptides covering the entire sequence of the GP protein (left graph) or the same pool without the region corresponding to the mucin-like domain. Analysis was performed before vaccination (D0) or 28 days after vaccination (D28) in the placebo group, as well as in the groups of people immunized with the ChAd3-EBOZ vaccine at low dose or high dose. Results highlight a benefit in terms of immunogenicity for removal of the mucin-like domain from the GP sequence.

FIG. 16: Viability assessments (upper) and ranked productivity (lower) on day 13 after starting a production culture and ranking of the expression levels of 5 leading clonally derived cell lines, when used under different, small scale (10 ml) culture conditions, suitable for eventual scale-up (lower). A total of 360 10-ml scale bioreactors were used to generate these results. Cultures were executed according to several simple fed-batch production concepts. Highest yielding clonal cell lines were taken into additional evaluation work and further cell line improvement activity.

FIG. 17: Viabilities (in %, top lines in graph) and viable cell density (in cells/ml, lower lines in graph) of two clonal CHO cell lines expressing the GP ΔTM-ΔMUC-variant protein in a simple fed-batch process.

FIG. 18: Secreted protein production kinetics for 13 days under production conditions from two clonal CHO cell lines expressing the GP ΔTM-ΔMUC-T4 variant protein using a simple fed-batch process.

FIG. 19: Western Blot of Histidine tag-containing and Histidine tag-free GP ΔTM-ΔMUC-T4 proteins, detected by the neutralizing (patient derived) KZ-52 antibody and following “native” non-denaturing gel electrophoresis.

FIG. 20: Analysis of a cell-free CHO supernatant containing Ebola GP_ΔTM-ΔMUC-T4 protein by Size Exclusion Chromatography (Trimer product is indicated by arrow at about 4.5 min). Insert shows more detail by signal amplification. Contaminants are found at 7 min or later.

FIG. 21: Purified GP_ΔTM-ΔMUC-T4 protein after Anionic Exchange Chromatography and Size Exclusion Chromatography.

### DETAILED DESCRIPTION

To be elicited in vivo in an appropriate manner, neutralizing antibodies require a suitable conformation of the provided protein, which remain difficult to be controlled with recombinant viruses. In this context, a vaccine based on a near-native, highly characterized and pure glycoprotein (GP) immunogen would be extremely promising to protect against Ebola disease through a recombinant protein-based vaccination strategy able to induce potent neutralizing antibodies. Given these observations and according to various aspects of the present disclosure, a near-native recombinant GP protein is a most promising antigen to develop a prophylactic vaccine able to elicit epitope-specific antibodies and protect against EBOV infection.

In the present disclosure, several variants of soluble and cell-secreted GP proteins, a total of 26 molecular variants, were engineered, including: a protein lacking the Transmembrane domain GP ΔTM protein, a protein lacking the mucin-like domain GP ΔMUC protein, a GP variant molecule with added trimerization motifs at the C-terminus and further variations and various combinations thereof. The variants were expressed using advanced methodologies combining superior expression vectors and gene transfer approaches for CHO cells combined with novel protein production approaches and using innovative bioreactors which are mixed by orbital shaking. [Matasci, M, et al. “The PiggyBac transposon enhances the frequency of CHO stable cell line generation and yields recombinant lines with superior productivity and stability.” Biotechnology and bioengineering 108.9 (2011): 2141-2150; De Jesus, M, and F M Wurm. “Manufacturing recombinant proteins in kg-ton quantities using animal cells in bioreactors.” European Journal of Pharmaceutics and Biopharmaceutics 78.2 (2011): 184-188; Tissot, S, et al. “kLa as a predictor for successful probe-independent mammalian cell bioprocesses in orbitally shaken bioreactors.” New biotechnology 29.3 (2012): 387-394; Reclari et al, Phys. Fluids 26, 052104, doi 10.1063/1.4874612, 2014, Zhu et al, Biotech Prog. Doi 10.1002/btpr 2375, 2016, Zhu et al, Biochem. Eng. J. doi.org/10.1016/j.beij.2017.]. These superior technologies allowed the rapid production of all desired variants of the Ebola GP in sufficient quantity and quality. Although vaccine research against Ebola disease is led by viral vector-based vaccines, some researchers have focused on the possibility to induce protection through the use of a recombinant GP protein. [Bengtsson et al., “Matrix-M adjuvant enhances antibody, cellular and protective immune responses of a Zaire Ebola/Makona virus glycoprotein (GP) nanoparticle vaccine in mice.” Vaccine 34.16 (2016): 1927-1935.] expressed a wild type full length Makona EBOV GP in insect cells with a recombinant baculovirus and entered a Phase I human clinical trial in response to the 2013-2016 EBOV epidemic. Other research groups proposed the mammalian cells expression to favor post-translational modifications and glycosylation [Konduru, K. et al. Against Lethal Challenge in Vaccinated Mice. 29, 2968-2977 (2012); Mellquist-Riemenschneider, J L., et al. “Comparison of the protective efficacy of DNA and baculovirus-derived protein vaccines for EBOLA virus in guinea pigs.” Virus research 92.2 (2003): 187-193.]. However, such vaccine design was not further developed, likely due to the difficulty of producing enough material of a good quality recombinant GP protein. Through the approaches disclosed herein, high-yielding and high-quality GPs were produced in suspension adapted CHO cells, retaining near-native structures, and exposing critical neutralizing epitopes for binding to monoclonal and neutralizing antibodies, even upon removal of the mucin-like domain. In particular, the inclusion of a trimerization motif resulted in proteins showing the highest breadth of reactivity with several conformational and neutralizing monoclonal antibodies.

Recombinant GP vaccines described herein overcome problems inherent to virus vector-based vaccines offering additional opportunities to design clinical trials with prime-boost regimens to obtain a stronger protection against Ebola infection. In addition, the compositions and methods described herein allow the control and characterization of the conformation adopted by the antigen, which is nearly impossible in the current vaccine candidates setting. Also, protein-based formulations can be stored in a liquid formulation at temperatures of 4° C. or can be lyophilized and thus allowing storage and transport at ambient temperatures, conditions which cannot be applied for virus-vector based vaccines, since they require holding such material at −80° C. or below.

Terms used herein generally have the meaning that scientists in the field would ascribe to them. The following definitions will assist understanding of the invention.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

An “analog,” such as a “variant” or a “derivative,” is a compound substantially similar in structure and having the same biological activity, albeit in certain instances to a differing degree, to a naturally-occurring molecule. For example, a polypeptide variant refers to a polypeptide sharing substantially similar structure and having the same biological activity as a reference polypeptide. Variants or analogs differ in the composition of their amino acid sequences compared to the naturally-occurring polypeptide from which the analog is derived, based on one or more mutations involving (i) deletion of one or more amino acid residues at one or more termini of the polypeptide and/or one or more internal regions of the naturally-occurring polypeptide sequence (e.g., fragments), (ii) insertion or addition of one or more amino acids at one or more termini (typically an “addition” or “fusion”) of the polypeptide and/or one or more internal regions (typically an “insertion”) of the naturally-occurring polypeptide sequence or (iii) substitution of one or more amino acids for other amino acids in the naturally-occurring polypeptide sequence. By way of example, a “derivative” is a type of analog and refers to a polypeptide sharing the same or substantially similar structure as a reference polypeptide that has been modified, e.g., chemically.

A variant polypeptide is a type of analog polypeptide and includes insertion variants, wherein one or more amino acid residues are added to a protein amino acid sequence of the present disclosure. Insertions may be located at either or both termini of the protein, and/or may be positioned within internal regions of the polypeptide amino acid sequence. Insertion variants, with additional residues at either or both termini, include for example, fusion proteins and proteins including amino acid tags or other amino acid labels.

In deletion variants, one or more amino acid residues in a polypeptide as described herein are removed. Deletions can be affected at one or both termini of the protein polypeptide, and/or with removal of one or more residues within the protein amino acid sequence. Deletion variants, therefore, include fragments of a protein polypeptide sequence.

In substitution variants, one or more amino acid residues of a protein polypeptide are removed and replaced with alternative residues. In one aspect, the substitutions are conservative in nature and conservative substitutions of this type are well known in the art. Alternatively, the present disclosure embraces substitutions that are also non-conservative. Exemplary conservative substitutions are described in Lehninger, [Biochemistry, 2nd Edition; Worth Publishers, Inc., New York (1975), pp. 71-77]

“Conservative amino acid substitution” refers to the interchange of a residue having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

The term “nucleic acid” refers to a single or double-stranded polymer of deoxyribonucleotide or ribonucleotide bases read from the 5′ to the 3′ end.

The term “encoding” refers to a polynucleotide sequence encoding one or more amino acids. The term does not require a start or stop codon. An amino acid sequence can be encoded in any one of six different reading frames provided by a double-stranded polynucleotide sequence. In some variations, encoding sequences further include a start and/or a stop codon.

A “vector” refers to a polynucleotide which, when independent of the host chromosome, is capable of replication in a host organism. Examples of vectors include plasmids. Vectors typically have an origin of replication. Vectors can comprise, e.g., transcription and translation terminators, transcription and translation initiation sequences, and promoters useful for regulation of the expression of the particular nucleic acid.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified and that retains the modification, such as a daughter cell. Thus, for example, recombinant cells express genes that are not found within the native (nonrecombinant) form of the cell or express native genes that are otherwise abnormally expressed, under-expressed or not expressed at all.

The terms “identical” or percent “identity,” in the context of two or more nucleic acids or polypeptide sequences, refer to two or more sequences or subsequences that are the same. “Substantially identical” refers to two or more nucleic acids or polypeptide sequences having a specified percentage (or specified minimum percentage) of amino acid residues or nucleotides that are the same (i.e., (at least) 60% identity, optionally 65%, 70%, 75%, 80%, 85%, 90%, or 95% identity over a specified region, or, when not specified, over the entire sequence), when compared and aligned for maximum correspondence over a comparison window, or designated region as measured using one of the sequence comparison algorithms below or by manual alignment and visual inspection. This definition also refers to the complement of a test sequence. Optionally, the identity or substantial identity exists over a region that is at least about 50 nucleotides in length, or more preferably over a region that is 100 to 500 or 1000 or more nucleotides or amino acids in length.

A “non-native amino acid” in a protein sequence refers to any amino acid other than the amino acid that occurs in the corresponding position in an alignment with a naturally-occurring polypeptide with the lowest smallest sum probability where the comparison window is the length of the monomer domain queried and when compared to a naturally-occurring sequence in the non-redundant (“nr”) database of Genbank using BLAST 2.0. BLAST 2.0 is described in the art [17], respectively. Software for performing BLAST analyses is publicly available through the National Center for Biotechnology Information.

The term “codon optimized” refers to the modification of the nucleotide sequence for a desired protein for expression whereby the resulting amino acid sequence of the desired protein remains unchanged, the use of codons for amino acids is however optimized for the use of codons in the respective expression host system.

The term “heterologous” with respect to a nucleic acid, or a polypeptide component, indicates that the component occurs where it is not normally found in nature (e.g., relative to an adjacent component) and/or that it originates from a different source or species.

An “effective amount” or a “sufficient amount” of a substance is that amount necessary to effect beneficial or desired results, including clinical results, and, as such, an “effective amount” depends upon the context in which it is being applied. In the context of administering an antigenic composition or vaccine composition, an effective amount contains sufficient antigen (e.g., an EBOV GP of the disclosure) to elicit an immune response. An effective amount can be administered in one or more doses. Efficacy can be shown in an experimental or clinical trial, for example, by comparing results achieved with a substance of interest compared to an experimental control.

The term “dose” as used herein in reference to an antigenic composition refers to a measured portion of the antigenic composition taken by (administered to or received by) a subject at any one time.

The term “about” as used herein in reference to a value, encompasses from 90% to 110% of that value (e.g., about 200 μg GP refers to 180 μg to 220 μg GP).

The term “vaccination” as used herein refers to the introduction of vaccine into a body of an organism.

A “subject” is a living multi-cellular vertebrate organism. In the context of this disclosure, the subject can be an experimental subject, such as a non-human mammal (e.g., a mouse, a rat, or a non-human primate). Alternatively, the subject can be a human subject.

An “antigenic composition” or “vaccine composition” is a composition of matter suitable for administration to a human or animal subject (e.g., in an experimental or clinical setting) that is capable of eliciting a specific immune response, e.g., against a pathogen, such as Ebola virus. As such, an antigenic composition includes one or more antigens (for example, peptide or polypeptide antigens) or antigenic epitopes. An antigenic composition or vaccine composition can also include one or more additional components capable of eliciting or enhancing an immune response, such as an excipient, carrier, and/or adjuvant. In certain aspects of this disclosure, antigenic compositions or vaccine compositions are administered to elicit an immune response that protects the subject against symptoms or conditions induced by a pathogen. In some cases, symptoms or disease caused by a pathogen is prevented (or reduced or ameliorated) by inhibiting replication of the pathogen (e.g., virus) following exposure of the subject to the pathogen. In one aspect of this disclosure, the term antigenic composition or vaccine composition will be understood to encompass compositions that are intended for administration to a subject or population of subjects for the purpose of eliciting a protective or palliative immune response against a virus.

“Adjuvant” refers to a substance which, when added to a composition comprising an antigen, nonspecifically enhances or potentiates an immune response to the antigen in the recipient upon exposure. Common adjuvants include suspensions of minerals (alum, aluminum hydroxide, aluminum phosphate) onto which an antigen is adsorbed; emulsions, including water-in-oil, and oil-in-water (and variants thereof, including double emulsions and reversible emulsions), liposaccharides, lipopolysaccharides, immunostimulatory nucleic acids (such as CpG oligonucleotides), liposomes, Pattern Recognition Receptor (PRR) agonists (e.g. NALP3. RIG-I-like receptors (RIG-I and MDA5), and Toll-like Receptor agonists (particularly, TLR2, TLR3, TLR4, TLR7/8 and TLR9 agonists)), and various combinations of such components.

An “immune response” is a response of a cell of the immune system, such as a B cell, T cell, or monocyte, to a stimulus, such as a pathogen or antigen (e.g., formulated as an antigenic composition or a vaccine). An immune response can be a B cell response, which results in the production of specific antibodies, such as antigen specific neutralizing antibodies. An immune response can also be a T cell response, such as a CD4+ response or a CD8+ response. B cell and T cell responses are aspects of a “cellular” immune response. An immune response can also be a “humoral” immune response, which is mediated by antibodies. In some cases, the response is specific for a particular antigen (that is, an “antigen-specific response”). If the antigen is derived from a pathogen, the antigen-specific response is a “pathogen-specific response.” A “protective immune response” is an immune response that inhibits a detrimental function or activity of a pathogen, reduces infection by a pathogen, or decreases symptoms (including death) that result from infection by the pathogen. A protective immune response can be measured, for example, by viral and immune assays using a serum sample from an immunized subject for testing the ability of serum antibodies for inhibition of viral replication, such as: plaque reduction neutralization test (PRNT), ELISA-neutralization assay, antibody dependent cell-mediated cytotoxicity assay (ADCC), complement-dependent cytotoxicity (CDC), antibody dependent cell-mediated phagocytosis (ADCP). In addition, vaccine efficacy can be tested by measuring the T cell response CD4+ and CD8+ after immunization, using flow cytometry (FACS) analysis or ELISpot assay. The protective immune response can be tested by measuring resistance to pathogen challenge in vivo in an animal model. In humans, a protective immune response can be demonstrated in a population study, comparing measurements of infection, symptoms, morbidity, mortality, etc. in treated subjects compared to untreated controls. Exposure of a subject to an immunogenic stimulus, such as a pathogen or antigen (e.g., formulated as an antigenic composition or vaccine), elicits a primary immune response specific for the stimulus, that is, the exposure “primes” the immune response. A subsequent exposure, e.g., by immunization, to the stimulus can increase or “boost” the magnitude (or duration, or both) of the specific immune response. Thus, “boosting” a preexisting immune response by administering an antigenic composition increases the magnitude of an antigen (or pathogen) specific response, (e.g., by increasing antibody titer and/or affinity, by increasing the frequency of antigen specific B or T cells, by inducing maturation effector function, or a combination thereof).

The phrase “specifically (or selectively) binds,” when referring to the interaction between an antibody or fragment thereof and a GP as disclosed herein, refers to a binding reaction that can be determinative of the presence of the polypeptide in a heterogeneous population of proteins (e.g., a cell or tissue lysate) and other biologics. Thus, under standard conditions used in antibody binding assays, the specified GP binds to a particular target antibody or fragment thereof above background (e.g., 2×, 5×, 10× or more above background) and does not bind in a significant amount to other molecules present in the sample.

As used herein, an “expression vector” is a DNA construct that contains a structural gene operably linked to an expression control sequence so that the structural gene can be expressed when the expression vector is transferred into an appropriate host cell. Two DNA sequences are said to be “operably linked” if the biological activity of one region will affect the other region and also if the nature of the linkage between the two DNA sequences does not (1) result in the introduction of a frame-shift mutation, (2) interfere with the ability of the promoter region sequence to direct the transcription of the desired sequence, or (3) interfere with the ability of the desired sequence to be transcribed by the promoter region sequence. Thus, a promoter region would be operably linked to a desired DNA sequence if the promoter were capable of effecting transcription of that desired DNA sequence. As described herein, vectors suitable for expression in all varieties of host cells are contemplated, including prokaryotic expression vectors and eukaryotic expression vectors. Exemplary eukaryotic expression vectors include vectors for expression in mammalian cells, avian cells, insect cells, amphibian cells, plant cells, and fungal cells, including yeast cells. In various aspects of the present disclosure, the host cell is a Chinese Hamster Ovary (CHO) cell.

Conventional or known techniques of molecular biology (including recombinant techniques), microbiology, cell biology, biochemistry and immunology can be used to implement many elements of the invention. Such techniques are not always described herein in detail because they are known and/or are explained fully in the literature, such as, Molecular Cloning: A Laboratory Manual, second edition (Sambrook et al., 1989); Current Protocols in Molecular Biology (Ausubel et al., eds., 1987); PCR: The Polymerase Chain Reaction, (Mullis et al., eds., 1994); Culture of Animal Cells: A Manual of Basic Technique (Freshney, 1987); Harlow et al., Antibodies: A Laboratory Manual (Harlow et al., 1988); and Current Protocols in Immunology (Coligan et al., eds., 1991).

As used in this specification and the appended claims, the singular forms “a,” “an,” and “the” include plural reference unless the context clearly dictates otherwise.

### Ebola Virus

“Ebola virus” or “EBOV” is a genus of the Filoviridae family, which is known to cause severe and rapidly progressing hemorrhagic fever. There are many different Ebola virus species and strains based on nucleotide sequence and outbreak location, for example, Zaire, Tai Forest (previously known as Cote d'Ivoire or Ivory Coast), Sudan, Reston, and Bundibugyo. The most lethal forms of the virus are the Zaire and Sudan strains. The Reston strain is the only strain known to infect only non-human primates. The term “Ebola virus” also includes variants of Ebola virus isolated from different Ebola virus isolates.

Ebola GP is a type I transmembrane protein of 676 amino acids. It is post-translationally cleaved by furin into two subunits (GP1 and GP2) linked by a disulfide bond and is inserted into the viral membrane. The complex of GP1 and GP2 is frequently referred to as GP1/2 or GP. While GP1 mediates attachment to host cells, GP2 is the responsible for fusion of viral and host cell membranes. In the EBOV surface, GP assembles into a trimer of GP1/2 heterodimers which are highly glycosylated and adopt a chalice-like shape. The glycosylation occurs in the mucin-like domains of each monomer, and forms a shield protecting the virus from antibody recognition [Lee et al, Nature, 454, 2008]

The term “Ebola virus infection”, or “EBOV infection”, as used herein refers to the severe hemorrhagic fever resulting from exposure to the virus, or to an infected animal, or to an infected human patient, or contact with the bodily fluids or tissues from an animal or human patient having an Ebola virus infection. The “symptoms associated with an Ebola virus infection” include fever, headache, fatigue, loss of appetite, myalgia, diarrhea, vomiting, abdominal pain, dehydration and unexplained bleeding.

### GP Constructs

A polypeptide or protein identical to or derived from an EBOV amino acid sequence is used in the constructs of the present disclosure. In certain aspects, to the extent that trimerization of non-EBOV proteins are of interest, the present disclosure also provides methods for trimerizing other protein antigens.

The following polypeptide sequences and corresponding DNA sequences are contemplated according to various aspects of the present disclosure:

GP ΔTM-X-HIS protein
(SEQ ID NO: 1)
MEFWLSWVFLVAILKGVQCVPLGVIHNSTLQVSDVDKLVCRDKLSSTNQLRSVGLNL
EGNGVATDVPSATKRWGFRSGVPPKVVNYEAGEWAENCYNLEIKKPDGSECLPAAPDGIRGF
PRCRYVHKVSGTGPCAGDFAFHKEGAFFLYDRLASTVIYRGTTFAEGVVAFLILPQAKKDFFSS
HPLREPVNATEDPSSGYYSTTIRYQATGFGTNETEYLFEVDNLTYVQLESRFTPQFLLQLNETIY
TSGKRSNTTGKLIWKVNPEIDTTIGEWAFWETKKNLTRKIRSEELSFTVVSNGAKNISGQSPART
SSDPGTNTTTEDHKIMASENSSAMVQVHSQGREAAVSHLTTLATISTSPQSLTTKPGPDNSTH
NTPVYKLDISEATQVEQHHRRTDNDSTASDTPSATTAAGPPKAENTNTSKSTDFLDPATTTSPQ
NHSETAGNNNTHHQDTGEESASSGKLGLITNTIAGVAGLITGGRRTRREAIVNAQPKCNPNLHY
WTTQDEGAAIGLAWIPYFGPAAEGIYIEGLMHNQDGLICGLRQLANETTQALQLFLRATTELRTF
SILNRKAIDFLLQRWGGTCHILGPDCCIEPHDWTKNITDKIDQIIHDFVDKTLPIEGRHHHHHH
GP ΔTM-X-HIS DNA
(SEQ ID NO: 2)
ATGGAGTTCTGGTTGTCTTGGGTGTTTCTCGTCGCTATTCTGAAGGGGGTGCAGT
GCGTGCCACTGGGAGTGATCCACAACAGCACTCTCCAAGTCTCCGACGTGGACAAGCTCG
TGTGCCGAGACAAGCTTTCCAGCACAAACCAGCTGCGGTCCGTGGGACTGAACCTGGAGG
GAAATGGCGTGGCCACCGATGTGCCGTCTGCAACCAAGAGATGGGGCTTCCGCTCGGGC
GTGCCCCCGAAAGTGGTCAACTACGAGGCCGGAGAATGGGCCGAAAACTGTTACAACCTG
GAGATCAAGAAGCCCGACGGTTCCGAATGCCTCCCTGCCGCCCCTGACGGAATCAGGGG
ATTCCCGAGATGCCGCTACGTGCACAAAGTGTCCGGGACTGGACCTTGTGCTGGAGATTT
CGCCTTCCACAAGGAGGGCGCCTTTTTCCTGTACGACCGGCTCGCATCCACCGTGATCTA
TAGAGGAACCACCTTCGCGGAAGGAGTGGTCGCCTTCCTGATCCTGCCGCAAGCCAAGAA
GGACTTCTTTAGCTCCCATCCACTGCGCGAGCCCGTGAATGCCACCGAAGATCCCTCGTC
CGGCTATTACTCCACAACCATCCGGTACCAAGCCACCGGGTTCGGTACCAACGAGACTGA
GTACCTGTTCGAAGTCGACAACCTGACTTACGTGCAGCTCGAATCGCGGTTCACGCCACA
GTTCCTCCTGCAACTCAACGAAACTATCTACACTAGCGGGAAACGCTCGAACACCACCGG
GAAGCTGATTTGGAAGGTCAACCCTGAAATCGACACCACCATCGGAGAGTGGGCTTTTTG
GGAGACTAAGAAGAACCTCACCCGGAAGATCAGATCCGAGGAATTGTCCTTCACTGTGGT
GTCCAACGGAGCGAAGAACATCAGCGGACAGTCCCCCGCACGGACCTCATCGGACCCGG
GAACCAACACCACCACCGAGGACCACAAGATCATGGCCTCGGAGAACTCATCCGCCATGG
TGCAAGTGCATAGCCAGGGCCGCGAAGCCGCCGTGTCCCACCTGACTACCCTGGCGACC
ATCAGCACCAGCCCGCAGTCGCTGACTACTAAGCCTGGGCCAGACAACAGCACCCACAAC
ACCCCTGTGTACAAGCTGGACATTTCCGAAGCTACTCAGGTCGAGCAGCATCACCGGCGG
ACCGACAACGATTCCACCGCTTCCGACACCCCCTCCGCCACCACCGCCGCCGGCCCGCC
GAAGGCCGAAAACACCAATACGTCAAAGTCGACCGATTTCCTTGACCCCGCCACCACTAC
GAGCCCCCAGAACCACTCAGAAACTGCGGGCAACAACAACACCCACCACCAGGATACTGG
AGAGGAGTCCGCATCAAGCGGAAAGCTCGGCCTGATTACTAACACTATCGCGGGGGTCGC
TGGATTGATTACCGGTGGTAGAAGGACCAGGAGAGAGGCCATTGTGAACGCGCAGCCGAA
GTGCAACCCTAATCTGCATTACTGGACCACTCAGGACGAGGGCGCCGCCATCGGCCTGGC
CTGGATTCCGTACTTCGGCCCGGCCGCGGAAGGCATATACATTGAGGGACTTATGCACAA
TCAGGATGGACTGATTTGTGGTCTGCGCCAGCTGGCCAACGAAACCACCCAGGCGCTGCA
GCTGTTCCTCCGGGCAACCACCGAACTTCGCACTTTCTCCATCCTGAACCGGAAGGCCATT
GACTTCCTCTTGCAACGCTGGGGAGGAACTTGCCACATCCTGGGTCCTGATTGCTGCATC
GAACCGCATGACTGGACAAAGAACATCACCGACAAAATCGACCAGATCATCCACGATTTCG
TGGACAAGACCCTGCCTATCGAGGGCCGGCACCATCACCACCACCATTAGTGA
GP ΔTM-ΔMUC-X-HIS protein
(SEQ ID NO: 3)
MEFWLSWVFLVAILKGVQCVPLGVIHNSTLQVSDVDKLVCRDKLSSTNQLRSVGLNL
EGNGVATDVPSATKRWGFRSGVPPKVVNYEAGEWAENCYNLEIKKPDGSECLPAAPDGIRGF
PRCRYVHKVSGTGPCAGDFAFHKEGAFFLYDRLASTVIYRGTTFAEGVVAFLILPQAKKDFFSS
HPLREPVNATEDPSSGYYSTTIRYQATGFGTNETEYLFEVDNLTYVQLESRFTPQFLLQLNETIY
TSGKRSNTTGKLIWKVNPEIDTTIGEWAFWETKKNLTRKIRSEELSFTVVNTHHQDTGEESASS
GKLGLITNTIAGVAGLITGGRRTRREAIVNAQPKCNPNLHYWTTQDEGAAIGLAWIPYFGPAAE
GIYIEGLMHNQDGLICGLRQLANETTQALQLFLRATTELRTFSILNRKAIDFLLQRWGGTCHILGP
DCCIEPHDWTKNITDKIDQIIHDFVDKTLPIEGRHHHHHH
GP ΔTM-ΔMUC-X-HIS DNA
(SEQ ID NO: 4)
ATGGAGTTCTGGTTGTCTTGGGTGTTTCTCGTCGCTATTCTGAAGGGGGTGCAGT
GCGTGCCACTGGGAGTGATCCACAACAGCACTCTCCAAGTCTCCGACGTGGACAAGCTCG
TGTGCCGAGACAAGCTTTCCAGCACAAACCAGCTGCGGTCCGTGGGACTGAACCTGGAGG
GAAATGGCGTGGCCACCGATGTGCCGTCTGCAACCAAGAGATGGGGCTTCCGCTCGGGC
GTGCCCCCGAAAGTGGTCAACTACGAGGCCGGAGAATGGGCCGAAAACTGTTACAACCTG
GAGATCAAGAAGCCCGACGGTTCCGAATGCCTCCCTGCCGCCCCTGACGGAATCAGGGG
ATTCCCGAGATGCCGCTACGTGCACAAAGTGTCCGGGACTGGACCTTGTGCTGGAGATTT
CGCCTTCCACAAGGAGGGCGCCTTTTTCCTGTACGACCGGCTCGCATCCACCGTGATCTA
TAGAGGAACCACCTTCGCGGAAGGAGTGGTCGCCTTCCTGATCCTGCCGCAAGCCAAGAA
GGACTTCTTTAGCTCCCATCCACTGCGCGAGCCCGTGAATGCCACCGAAGATCCCTCGTC
CGGCTATTACTCCACAACCATCCGGTACCAAGCCACCGGGTTCGGTACCAACGAGACTGA
GTACCTGTTCGAAGTCGACAACCTGACTTACGTGCAGCTCGAATCGCGGTTCACGCCACA
GTTCCTCCTGCAACTCAACGAAACTATCTACACTAGCGGGAAACGCTCGAACACCACCGG
GAAGCTGATTTGGAAGGTCAACCCTGAAATCGACACCACCATCGGAGAGTGGGCTTTTTG
GGAGACTAAGAAGAACCTCACCCGGAAGATCAGATCCGAGGAATTGTCCTTCACCGTGGT
CAACACCCACCACCAGGATACTGGAGAGGAGTCCGCATCAAGCGGAAAGCTCGGCCTGAT
TACTAACACTATCGCGGGGGTCGCTGGATTGATTACCGGTGGTAGAAGGACCAGGAGAGA
GGCCATTGTGAACGCGCAGCCGAAGTGCAACCCTAATCTGCATTACTGGACCACTCAGGA
CGAGGGCGCCGCCATCGGCCTGGCCTGGATTCCGTACTTCGGCCCGGCCGCGGAAGGC
ATATACATTGAGGGACTTATGCACAATCAGGATGGACTGATTTGTGGTCTGCGCCAGCTGG
CCAACGAAACCACCCAGGCGCTGCAGCTGTTCCTCCGGGCAACCACCGAACTTCGCACTT
TCTCCATCCTGAACCGGAAGGCCATTGACTTCCTCTTGCAACGCTGGGGAGGAACTTGCC
ACATCCTGGGTCCTGATTGCTGCATCGAACCGCATGACTGGACAAAGAACATCACCGACAA
AATCGACCAGATCATCCACGATTTCGTGGACAAGACCCTGCCTATCGAGGGCCGGCACCA
TCACCACCACCATTAGTGA
GP ΔTM-ΔMUC-X-T4-HIS protein
(SEQ ID NO: 5)
MEFWLSWVFLVAILKGVQCVPLGVIHNSTLQVSDVDKLVCRDKLSSTNQLRSVGLNL
EGNGVATDVPSATKRWGFRSGVPPKVVNYEAGEWAENCYNLEIKKPDGSECLPAAPDGIRGF
PRCRYVHKVSGTGPCAGDFAFHKEGAFFLYDRLASTVIYRGTTFAEGVVAFLILPQAKKDFFSS
HPLREPVNATEDPSSGYYSTTIRYQATGFGTNETEYLFEVDNLTYVQLESRFTPQFLLQLNETIY
TSGKRSNTTGKLIWKVNPEIDTTIGEWAFWETKKNLTRKIRSEELSFTVVNTHHQDTGEESASS
GKLGLITNTIAGVAGLITGGRRTRREAIVNAQPKCNPNLHYWTTQDEGAAIGLAWIPYFGPAAE
GIYIEGLMHNQDGLICGLRQLANETTQALQLFLRATTELRTFSILNRKAIDFLLQRWGGTCHILGP
DCCIEPHDWTKNITDKIDQIIHDFVDKTLPIEGRGSGYIPEAPRDGQAYVRKDGEWVLLSTFLGT
HHHHHH
GP ΔTM-ΔMUC-X-T4-HIS DNA
(SEQ ID NO: 6)
ATGGAGTTCTGGTTGTCTTGGGTGTTTCTCGTCGCTATTCTGAAGGGGGTGCAGT
GCGTGCCACTGGGAGTGATCCACAACAGCACTCTCCAAGTCTCCGACGTGGACAAGCTCG
TGTGCCGAGACAAGCTTTCCAGCACAAACCAGCTGCGGTCCGTGGGACTGAACCTGGAGG
GAAATGGCGTGGCCACCGATGTGCCGTCTGCAACCAAGAGATGGGGCTTCCGCTCGGGC
GTGCCCCCGAAAGTGGTCAACTACGAGGCCGGAGAATGGGCCGAAAACTGTTACAACCTG
GAGATCAAGAAGCCCGACGGTTCCGAATGCCTCCCTGCCGCCCCTGACGGAATCAGGGG
ATTCCCGAGATGCCGCTACGTGCACAAAGTGTCCGGGACTGGACCTTGTGCTGGAGATTT
CGCCTTCCACAAGGAGGGCGCCTTTTTCCTGTACGACCGGCTCGCATCCACCGTGATCTA
TAGAGGAACCACCTTCGCGGAAGGAGTGGTCGCCTTCCTGATCCTGCCGCAAGCCAAGAA
GGACTTCTTTAGCTCCCATCCACTGCGCGAGCCCGTGAATGCCACCGAAGATCCCTCGTC
CGGCTATTACTCCACAACCATCCGGTACCAAGCCACCGGGTTCGGTACCAACGAGACTGA
GTACCTGTTCGAAGTCGACAACCTGACTTACGTGCAGCTCGAATCGCGGTTCACGCCACA
GTTCCTCCTGCAACTCAACGAAACTATCTACACTAGCGGGAAACGCTCGAACACCACCGG
GAAGCTGATTTGGAAGGTCAACCCTGAAATCGACACCACCATCGGAGAGTGGGCTTTTTG
GGAGACTAAGAAGAACCTCACCCGGAAGATCAGATCCGAGGAATTGTCCTTCACCGTGGT
CAACACCCACCACCAGGATACTGGAGAGGAGTCCGCATCAAGCGGAAAGCTCGGCCTGAT
TACTAACACTATCGCGGGGGTCGCTGGATTGATTACCGGTGGTAGAAGGACCAGGAGAGA
GGCCATTGTGAACGCGCAGCCGAAGTGCAACCCTAATCTGCATTACTGGACCACTCAGGA
CGAGGGCGCCGCCATCGGCCTGGCCTGGATTCCGTACTTCGGCCCGGCCGCGGAAGGC
ATATACATTGAGGGACTTATGCACAATCAGGATGGACTGATTTGTGGTCTGCGCCAGCTGG
CCAACGAAACCACCCAGGCGCTGCAGCTGTTCCTCCGGGCAACCACCGAACTTCGCACTT
TCTCCATCCTGAACCGGAAGGCCATTGACTTCCTCTTGCAACGCTGGGGAGGAACTTGCC
ACATCCTGGGTCCTGATTGCTGCATCGAACCGCATGACTGGACAAAGAACATCACCGACAA
AATCGACCAGATCATCCACGATTTCGTGGACAAGACCCTGCCTATCGAAGGGCGCGGATC
AGGTTACATCCCCGAGGCCCCGAGAGACGGCCAGGCCTACGTGCGGAAGGACGGCGAAT
GGGTCCTCCTGTCCACCTTCCTTGGGACTCACCATCACCACCACCATTGATAA
GP ΔTM-ΔMUC-X-GCN4-HIS protein
(SEQ ID NO: 7)
MEFWLSWVFLVAILKGVQCVPLGVIHNSTLQVSDVDKLVCRDKLSSTNQLRSVGLNL
EGNGVATDVPSATKRWGFRSGVPPKVVNYEAGEWAENCYNLEIKKPDGSECLPAAPDGIRGF
PRCRYVHKVSGTGPCAGDFAFHKEGAFFLYDRLASTVIYRGTTFAEGVVAFLILPQAKKDFFSS
HPLREPVNATEDPSSGYYSTTIRYQATGFGTNETEYLFEVDNLTYVQLESRFTPQFLLQLNETIY
TSGKRSNTTGKLIWKVNPEIDTTIGEWAFWETKKNLTRKIRSEELSFTVVNTHHQDTGEESASS
GKLGLITNTIAGVAGLITGGRRTRREAIVNAQPKCNPNLHYWTTQDEGAAIGLAWIPYFGPAAE
GIYIEGLMHNQDGLICGLRQLANETTQALQLFLRATTELRTFSILNRKAIDFLLQRWGGTCHILGP
DCCIEPHDWTKNITDKIDQIIHDFVDKTLPIEGRGSGKQIEDKIEEILSKIYHIENEIARIKKLIGHHH
HHH
GP ΔTM-ΔMUC-X-GCN4-HIS DNA
(SEQ ID NO: 8)
ATGGAGTTCTGGTTGTCTTGGGTGTTTCTCGTCGCTATTCTGAAGGGGGTGCAGT
GCGTGCCACTGGGAGTGATCCACAACAGCACTCTCCAAGTCTCCGACGTGGACAAGCTCG
TGTGCCGAGACAAGCTTTCCAGCACAAACCAGCTGCGGTCCGTGGGACTGAACCTGGAGG
GAAATGGCGTGGCCACCGATGTGCCGTCTGCAACCAAGAGATGGGGCTTCCGCTCGGGC
GTGCCCCCGAAAGTGGTCAACTACGAGGCCGGAGAATGGGCCGAAAACTGTTACAACCTG
GAGATCAAGAAGCCCGACGGTTCCGAATGCCTCCCTGCCGCCCCTGACGGAATCAGGGG
ATTCCCGAGATGCCGCTACGTGCACAAAGTGTCCGGGACTGGACCTTGTGCTGGAGATTT
CGCCTTCCACAAGGAGGGCGCCTTTTTCCTGTACGACCGGCTCGCATCCACCGTGATCTA
TAGAGGAACCACCTTCGCGGAAGGAGTGGTCGCCTTCCTGATCCTGCCGCAAGCCAAGAA
GGACTTCTTTAGCTCCCATCCACTGCGCGAGCCCGTGAATGCCACCGAAGATCCCTCGTC
CGGCTATTACTCCACAACCATCCGGTACCAAGCCACCGGGTTCGGTACCAACGAGACTGA
GTACCTGTTCGAAGTCGACAACCTGACTTACGTGCAGCTCGAATCGCGGTTCACGCCACA
GTTCCTCCTGCAACTCAACGAAACTATCTACACTAGCGGGAAACGCTCGAACACCACCGG
GAAGCTGATTTGGAAGGTCAACCCTGAAATCGACACCACCATCGGAGAGTGGGCTTTTTG
GGAGACTAAGAAGAACCTCACCCGGAAGATCAGATCCGAGGAATTGTCCTTCACCGTGGT
CAACACCCACCACCAGGATACTGGAGAGGAGTCCGCATCAAGCGGAAAGCTCGGCCTGAT
TACTAACACTATCGCGGGGGTCGCTGGATTGATTACCGGTGGTAGAAGGACCAGGAGAGA
GGCCATTGTGAACGCGCAGCCGAAGTGCAACCCTAATCTGCATTACTGGACCACTCAGGA
CGAGGGCGCCGCCATCGGCCTGGCCTGGATTCCGTACTTCGGCCCGGCCGCGGAAGGC
ATATACATTGAGGGACTTATGCACAATCAGGATGGACTGATTTGTGGTCTGCGCCAGCTGG
CCAACGAAACCACCCAGGCGCTGCAGCTGTTCCTCCGGGCAACCACCGAACTTCGCACTT
TCTCCATCCTGAACCGGAAGGCCATTGACTTCCTCTTGCAACGCTGGGGAGGAACTTGCC
ACATCCTGGGTCCTGATTGCTGCATCGAACCGCATGACTGGACAAAGAACATCACCGACAA
AATCGACCAGATCATCCACGATTTCGTGGACAAGACCCTGCCTATCGAGGGCCGGGGGAG
CGGCAAGCAAATCGAAGATAAAATCGAGGAAATTCTGTCAAAGATTTACCACATTGAGAAC
GAAATCGCCCGGATCAAGAAGCTGATAGGTCATCACCACCACCATCATTGATAA
GP ΔTM-ΔFUR-X-HIS protein
(SEQ ID NO: 9)
MEFWLSWVFLVAILKGVQCVPLGVIHNSTLQVSDVDKLVCRDKLSSTNQLRSVGLNL
EGNGVATDVPSATKRWGFRSGVPPKVVNYEAGEWAENCYNLEIKKPDGSECLPAAPDGIRGF
PRCRYVHKVSGTGPCAGDFAFHKEGAFFLYDRLASTVIYRGTTFAEGVVAFLILPQAKKDFFSS
HPLREPVNATEDPSSGYYSTTIRYQATGFGTNETEYLFEVDNLTYVQLESRFTPQFLLQLNETIY
TSGKRSNTTGKLIWKVNPEIDTTIGEWAFWETKKNLTRKIRSEELSFTVVSNGAKNISGQSPART
SSDPGTNTTTEDHKIMASENSSAMVQVHSQGREAAVSHLTTLATISTSPQSLTTKPGPDNSTH
NTPVYKLDISEATQVEQHHRRTDNDSTASDTPSATTAAGPPKAENTNTSKSTDFLDPATTTSPQ
NHSETAGNNNTHHQDTGEESASSGKLGLITNTIAGVAGLITGGAGTAAEAIVNAQPKCNPNLHY
WTTQDEGAAIGLAWIPYFGPAAEGIYIEGLMHNQDGLICGLRQLANETTQALQLFLRATTELRTF
SILNRKAIDFLLQRWGGTCHILGPDCCIEPHDWTKNITDKIDQIIHDFVDKTLPIEGRHHHHHH
GP ΔTM-ΔFUR-X-HIS DNA
(SEQ ID NO: 10)
ATGGAGTTCTGGTTGTCTTGGGTGTTTCTCGTCGCTATTCTGAAGGGGGTGCAGT
GCGTGCCACTGGGAGTGATCCACAACAGCACTCTCCAAGTCTCCGACGTGGACAAGCTCG
TGTGCCGAGACAAGCTTTCCAGCACAAACCAGCTGCGGTCCGTGGGACTGAACCTGGAGG
GAAATGGCGTGGCCACCGATGTGCCGTCTGCAACCAAGAGATGGGGCTTCCGCTCGGGC
GTGCCCCCGAAAGTGGTCAACTACGAGGCCGGAGAATGGGCCGAAAACTGTTACAACCTG
GAGATCAAGAAGCCCGACGGTTCCGAATGCCTCCCTGCCGCCCCTGACGGAATCAGGGG
ATTCCCGAGATGCCGCTACGTGCACAAAGTGTCCGGGACTGGACCTTGTGCTGGAGATTT
CGCCTTCCACAAGGAGGGCGCCTTTTTCCTGTACGACCGGCTCGCATCCACCGTGATCTA
TAGAGGAACCACCTTCGCGGAAGGAGTGGTCGCCTTCCTGATCCTGCCGCAAGCCAAGAA
GGACTTCTTTAGCTCCCATCCACTGCGCGAGCCCGTGAATGCCACCGAAGATCCCTCGTC
CGGCTATTACTCCACAACCATCCGGTACCAAGCCACCGGGTTCGGTACCAACGAGACTGA
GTACCTGTTCGAAGTCGACAACCTGACTTACGTGCAGCTCGAATCGCGGTTCACGCCACA
GTTCCTCCTGCAACTCAACGAAACTATCTACACTAGCGGGAAACGCTCGAACACCACCGG
GAAGCTGATTTGGAAGGTCAACCCTGAAATCGACACCACCATCGGAGAGTGGGCTTTTTG
GGAGACTAAGAAGAACCTCACCCGGAAGATCAGATCCGAGGAATTGTCCTTCACTGTGGT
GTCCAACGGAGCGAAGAACATCAGCGGACAGTCCCCCGCACGGACCTCATCGGACCCGG
GAACCAACACCACCACCGAGGACCACAAGATCATGGCCTCGGAGAACTCATCCGCCATGG
TGCAAGTGCATAGCCAGGGCCGCGAAGCCGCCGTGTCCCACCTGACTACCCTGGCGACC
ATCAGCACCAGCCCGCAGTCGCTGACTACTAAGCCTGGGCCAGACAACAGCACCCACAAC
ACCCCTGTGTACAAGCTGGACATTTCCGAAGCTACTCAGGTCGAGCAGCATCACCGGCGG
ACCGACAACGATTCCACCGCTTCCGACACCCCCTCCGCCACCACCGCCGCCGGCCCGCC
GAAGGCCGAAAACACCAATACGTCAAAGTCGACCGATTTCCTTGACCCCGCCACCACTAC
GAGCCCCCAGAACCACTCAGAAACTGCGGGCAACAACAACACCCACCACCAGGATACTGG
AGAGGAGTCCGCATCAAGCGGAAAGCTCGGCCTGATTACTAACACTATCGCGGGGGTCGC
TGGATTGATTACCGGTGGTGCCGGGACCGCGGCAGAGGCCATTGTGAACGCGCAGCCGA
AGTGCAACCCTAATCTGCATTACTGGACCACTCAGGACGAGGGCGCCGCCATCGGCCTGG
CCTGGATTCCGTACTTCGGCCCGGCCGCGGAAGGCATATACATTGAGGGACTTATGCACA
ATCAGGATGGACTGATTTGTGGTCTGCGCCAGCTGGCCAACGAAACCACCCAGGCGCTGC
AGCTGTTCCTCCGGGCAACCACCGAACTTCGCACTTTCTCCATCCTGAACCGGAAGGCCA
TTGACTTCCTCTTGCAACGCTGGGGAGGAACTTGCCACATCCTGGGTCCTGATTGCTGCAT
CGAACCGCATGACTGGACAAAGAACATCACCGACAAAATCGACCAGATCATCCACGATTTC
GTGGACAAGACCCTGCCTATCGAGGGCCGGCACCATCACCACCACCATTAGTGA
GP ΔTM-ΔMUC-ΔFUR-X-HIS protein
(SEQ ID NO: 11)
MEFWLSWVFLVAILKGVQCVPLGVIHNSTLQVSDVDKLVCRDKLSSTNQLRSVGLNL
EGNGVATDVPSATKRWGFRSGVPPKVVNYEAGEWAENCYNLEIKKPDGSECLPAAPDGIRGF
PRCRYVHKVSGTGPCAGDFAFHKEGAFFLYDRLASTVIYRGTTFAEGVVAFLILPQAKKDFFSS
HPLREPVNATEDPSSGYYSTTIRYQATGFGTNETEYLFEVDNLTYVQLESRFTPQFLLQLNETIY
TSGKRSNTTGKLIWKVNPEIDTTIGEWAFWETKKNLTRKIRSEELSFTVVNTHHQDTGEESASS
GKLGLITNTIAGVAGLITGGAGTAAAEAIVNAQPKCNPNLHYWTTQDEGAAIGLAWIPYFGPAAE
GIYIEGLMHNQDGLICGLRQLANETTQALQLFLRATTELRTFSILNRKAIDFLLQRWGGTCHILGP
DCCIEPHDWTKNITDKIDQIIHDFVDKTLPIEGRHHHHHH
GP ΔTM-ΔMUC-ΔFUR-X-HIS DNA
(SEQ ID NO: 12)
ATGGAGTTCTGGTTGTCTTGGGTGTTTCTCGTCGCTATTCTGAAGGGGGTGCAGT
GCGTGCCACTGGGAGTGATCCACAACAGCACTCTCCAAGTCTCCGACGTGGACAAGCTCG
TGTGCCGAGACAAGCTTTCCAGCACAAACCAGCTGCGGTCCGTGGGACTGAACCTGGAGG
GAAATGGCGTGGCCACCGATGTGCCGTCTGCAACCAAGAGATGGGGCTTCCGCTCGGGC
GTGCCCCCGAAAGTGGTCAACTACGAGGCCGGAGAATGGGCCGAAAACTGTTACAACCTG
GAGATCAAGAAGCCCGACGGTTCCGAATGCCTCCCTGCCGCCCCTGACGGAATCAGGGG
ATTCCCGAGATGCCGCTACGTGCACAAAGTGTCCGGGACTGGACCTTGTGCTGGAGATTT
CGCCTTCCACAAGGAGGGCGCCTTTTTCCTGTACGACCGGCTCGCATCCACCGTGATCTA
TAGAGGAACCACCTTCGCGGAAGGAGTGGTCGCCTTCCTGATCCTGCCGCAAGCCAAGAA
GGACTTCTTTAGCTCCCATCCACTGCGCGAGCCCGTGAATGCCACCGAAGATCCCTCGTC
CGGCTATTACTCCACAACCATCCGGTACCAAGCCACCGGGTTCGGTACCAACGAGACTGA
GTACCTGTTCGAAGTCGACAACCTGACTTACGTGCAGCTCGAATCGCGGTTCACGCCACA
GTTCCTCCTGCAACTCAACGAAACTATCTACACTAGCGGGAAACGCTCGAACACCACCGG
GAAGCTGATTTGGAAGGTCAACCCTGAAATCGACACCACCATCGGAGAGTGGGCTTTTTG
GGAGACTAAGAAGAACCTCACCCGGAAGATCAGATCCGAGGAATTGTCCTTCACCGTGGT
CAACACCCACCACCAGGATACTGGAGAGGAGTCCGCATCAAGCGGAAAGCTCGGCCTGAT
TACTAACACTATCGCGGGGGTCGCTGGATTGATTACCGGTGGTGCCGGGACCGCCGCGG
CAGAGGCCATTGTGAACGCGCAGCCGAAGTGCAACCCTAATCTGCATTACTGGACCACTC
AGGACGAGGGCGCCGCCATCGGCCTGGCCTGGATTCCGTACTTCGGCCCGGCCGCGGAA
GGCATATACATTGAGGGACTTATGCACAATCAGGATGGACTGATTTGTGGTCTGCGCCAGC
TGGCCAACGAAACCACCCAGGCGCTGCAGCTGTTCCTCCGGGCAACCACCGAACTTCGCA
CTTTCTCCATCCTGAACCGGAAGGCCATTGACTTCCTCTTGCAACGCTGGGGAGGAACTTG
CCACATCCTGGGTCCTGATTGCTGCATCGAACCGCATGACTGGACAAAGAACATCACCGA
CAAAATCGACCAGATCATCCACGATTTCGTGGACAAGACCCTGCCTATCGAGGGCCGGCA
CCATCACCACCACCATTAGTGA
GP ΔTM protein
(SEQ ID NO: 13)
MEFWLSWVFLVAILKGVQCVPLGVIHNSTLQVSDVDKLVCRDKLSSTNQLRSVGLNL
EGNGVATDVPSATKRWGFRSGVPPKVVNYEAGEWAENCYNLEIKKPDGSECLPAAPDGIRGF
PRCRYVHKVSGTGPCAGDFAFHKEGAFFLYDRLASTVIYRGTTFAEGVVAFLILPQAKKDFFSS
HPLREPVNATEDPSSGYYSTTIRYQATGFGTNETEYLFEVDNLTYVQLESRFTPQFLLQLNETIY
TSGKRSNTTGKLIWKVNPEIDTTIGEWAFWETKKNLTRKIRSEELSFTVVSNGAKNISGQSPART
SSDPGTNTTTEDHKIMASENSSAMVQVHSQGREAAVSHLTTLATISTSPQSLTTKPGPDNSTH
NTPVYKLDISEATQVEQHHRRTDNDSTASDTPSATTAAGPPKAENTNTSKSTDFLDPATTTSPQ
NHSETAGNNNTHHQDTGEESASSGKLGLITNTIAGVAGLITGGRRTRREAIVNAQPKCNPNLHY
WTTQDEGAAIGLAWIPYFGPAAEGIYIEGLMHNQDGLICGLRQLANETTQALQLFLRATTELRTF
SILNRKAIDFLLQRWGGTCHILGPDCCIEPHDWTKNITDKIDQIIHDFVDKTLP
GP ΔTM DNA
(SEQ ID NO: 14)
ATGGAGTTCTGGTTGTCTTGGGTGTTTCTCGTCGCTATTCTGAAGGGGGTGCAGT
GCGTGCCACTGGGAGTGATCCACAACAGCACTCTCCAAGTCTCCGACGTGGACAAGCTCG
TGTGCCGAGACAAGCTTTCCAGCACAAACCAGCTGCGGTCCGTGGGACTGAACCTGGAGG
GAAATGGCGTGGCCACCGATGTGCCGTCTGCAACCAAGAGATGGGGCTTCCGCTCGGGC
GTGCCCCCGAAAGTGGTCAACTACGAGGCCGGAGAATGGGCCGAAAACTGTTACAACCTG
GAGATCAAGAAGCCCGACGGTTCCGAATGCCTCCCTGCCGCCCCTGACGGAATCAGGGG
ATTCCCGAGATGCCGCTACGTGCACAAAGTGTCCGGGACTGGACCTTGTGCTGGAGATTT
CGCCTTCCACAAGGAGGGCGCCTTTTTCCTGTACGACCGGCTCGCATCCACCGTGATCTA
TAGAGGAACCACCTTCGCGGAAGGAGTGGTCGCCTTCCTGATCCTGCCGCAAGCCAAGAA
GGACTTCTTTAGCTCCCATCCACTGCGCGAGCCCGTGAATGCCACCGAAGATCCCTCGTC
CGGCTATTACTCCACAACCATCCGGTACCAAGCCACCGGGTTCGGTACCAACGAGACTGA
GTACCTGTTCGAAGTCGACAACCTGACTTACGTGCAGCTCGAATCGCGGTTCACGCCACA
GTTCCTCCTGCAACTCAACGAAACTATCTACACTAGCGGGAAACGCTCGAACACCACCGG
GAAGCTGATTTGGAAGGTCAACCCTGAAATCGACACCACCATCGGAGAGTGGGCTTTTTG
GGAGACTAAGAAGAACCTCACCCGGAAGATCAGATCCGAGGAATTGTCCTTCACTGTGGT
GTCCAACGGAGCGAAGAACATCAGCGGACAGTCCCCCGCACGGACCTCATCGGACCCGG
GAACCAACACCACCACCGAGGACCACAAGATCATGGCCTCGGAGAACTCATCCGCCATGG
TGCAAGTGCATAGCCAGGGCCGCGAAGCCGCCGTGTCCCACCTGACTACCCTGGCGACC
ATCAGCACCAGCCCGCAGTCGCTGACTACTAAGCCTGGGCCAGACAACAGCACCCACAAC
ACCCCTGTGTACAAGCTGGACATTTCCGAAGCTACTCAGGTCGAGCAGCATCACCGGCGG
ACCGACAACGATTCCACCGCTTCCGACACCCCCTCCGCCACCACCGCCGCCGGCCCGCC
GAAGGCCGAAAACACCAATACGTCAAAGTCGACCGATTTCCTTGACCCCGCCACCACTAC
GAGCCCCCAGAACCACTCAGAAACTGCGGGCAACAACAACACCCACCACCAGGATACTGG
AGAGGAGTCCGCATCAAGCGGAAAGCTCGGCCTGATTACTAACACTATCGCGGGGGTCGC
TGGATTGATTACCGGTGGTAGAAGGACCAGGAGAGAGGCCATTGTGAACGCGCAGCCGAA
GTGCAACCCTAATCTGCATTACTGGACCACTCAGGACGAGGGCGCCGCCATCGGCCTGGC
CTGGATTCCGTACTTCGGCCCGGCCGCGGAAGGCATATACATTGAGGGACTTATGCACAA
TCAGGATGGACTGATTTGTGGTCTGCGCCAGCTGGCCAACGAAACCACCCAGGCGCTGCA
GCTGTTCCTCCGGGCAACCACCGAACTTCGCACTTTCTCCATCCTGAACCGGAAGGCCATT
GACTTCCTCTTGCAACGCTGGGGAGGAACTTGCCACATCCTGGGTCCTGATTGCTGCATC
GAACCGCATGACTGGACAAAGAACATCACCGACAAAATCGACCAGATCATCCACGATTTCG
TGGACAAGACCCTGCCTTAGTGA
GP ΔTM-ΔMUC protein
(SEQ ID NO: 15)
MEFWLSWVFLVAILKGVQCVPLGVIHNSTLQVSDVDKLVCRDKLSSTNQLRSVGLNL
EGNGVATDVPSATKRWGFRSGVPPKVVNYEAGEWAENCYNLEIKKPDGSECLPAAPDGIRGF
PRCRYVHKVSGTGPCAGDFAFHKEGAFFLYDRLASTVIYRGTTFAEGVVAFLILPQAKKDFFSS
HPLREPVNATEDPSSGYYSTTIRYQATGFGTNETEYLFEVDNLTYVQLESRFTPQFLLQLNETIY
TSGKRSNTTGKLIWKVNPEIDTTIGEWAFWETKKNLTRKIRSEELSFTVVNTHHQDTGEESASS
GKLGLITNTIAGVAGLITGGRRTRREAIVNAQPKCNPNLHYWTTQDEGAAIGLAWIPYFGPAAE
GIYIEGLMHNQDGLICGLRQLANETTQALQLFLRATTELRTFSILNRKAIDFLLQRWGGTCHILGP
DCCIEPHDWTKNITDKIDQIIHDFVDKTLP
GP ΔTM-ΔMUC DNA
(SEQ ID NO: 16)
ATGGAGTTCTGGTTGTCTTGGGTGTTTCTCGTCGCTATTCTGAAGGGGGTGCAGT
GCGTGCCACTGGGAGTGATCCACAACAGCACTCTCCAAGTCTCCGACGTGGACAAGCTCG
TGTGCCGAGACAAGCTTTCCAGCACAAACCAGCTGCGGTCCGTGGGACTGAACCTGGAGG
GAAATGGCGTGGCCACCGATGTGCCGTCTGCAACCAAGAGATGGGGCTTCCGCTCGGGC
GTGCCCCCGAAAGTGGTCAACTACGAGGCCGGAGAATGGGCCGAAAACTGTTACAACCTG
GAGATCAAGAAGCCCGACGGTTCCGAATGCCTCCCTGCCGCCCCTGACGGAATCAGGGG
ATTCCCGAGATGCCGCTACGTGCACAAAGTGTCCGGGACTGGACCTTGTGCTGGAGATTT
CGCCTTCCACAAGGAGGGCGCCTTTTTCCTGTACGACCGGCTCGCATCCACCGTGATCTA
TAGAGGAACCACCTTCGCGGAAGGAGTGGTCGCCTTCCTGATCCTGCCGCAAGCCAAGAA
GGACTTCTTTAGCTCCCATCCACTGCGCGAGCCCGTGAATGCCACCGAAGATCCCTCGTC
CGGCTATTACTCCACAACCATCCGGTACCAAGCCACCGGGTTCGGTACCAACGAGACTGA
GTACCTGTTCGAAGTCGACAACCTGACTTACGTGCAGCTCGAATCGCGGTTCACGCCACA
GTTCCTCCTGCAACTCAACGAAACTATCTACACTAGCGGGAAACGCTCGAACACCACCGG
GAAGCTGATTTGGAAGGTCAACCCTGAAATCGACACCACCATCGGAGAGTGGGCTTTTTG
GGAGACTAAGAAGAACCTCACCCGGAAGATCAGATCCGAGGAATTGTCCTTCACCGTGGT
CAACACCCACCACCAGGATACTGGAGAGGAGTCCGCATCAAGCGGAAAGCTCGGCCTGAT
TACTAACACTATCGCGGGGGTCGCTGGATTGATTACCGGTGGTAGAAGGACCAGGAGAGA
GGCCATTGTGAACGCGCAGCCGAAGTGCAACCCTAATCTGCATTACTGGACCACTCAGGA
CGAGGGCGCCGCCATCGGCCTGGCCTGGATTCCGTACTTCGGCCCGGCCGCGGAAGGC
ATATACATTGAGGGACTTATGCACAATCAGGATGGACTGATTTGTGGTCTGCGCCAGCTGG
CCAACGAAACCACCCAGGCGCTGCAGCTGTTCCTCCGGGCAACCACCGAACTTCGCACTT
TCTCCATCCTGAACCGGAAGGCCATTGACTTCCTCTTGCAACGCTGGGGAGGAACTTGCC
ACATCCTGGGTCCTGATTGCTGCATCGAACCGCATGACTGGACAAAGAACATCACCGACAA
AATCGACCAGATCATCCACGATTTCGTGGACAAGACCCTGCCTTAGTGA
GP ΔTM-CC1-X-HIS
(SEQ ID NO: 17)
MEFWLSWVFLVAILKGVQCVPLGVIHNSTLQVSDVDKLVCRDKLSSTNQLRSVGLNL
EGNGVATDVPSATKRWGFRSGVPPKVVNYEAGEWAENCYNLEIKKPDGSECLPAAPDGIRGF
PRCRYVHKVSGTGPCAGDFAFHKEGAFFLYDRLASTVIYRGTTFAEGCVAFLILPQAKKDFFSS
HPLREPVNATEDPSSGYYSTTIRYQATGFGTNETEYLFEVDNLTYVQLESRFTPQFLLQLNETIY
TSGKRSNTTGKLIWKVNPEIDTTIGEWAFWETKKNLTRKIRSEELSFTVVNTHHQDTGEESASS
GKLGLITNTIAGVAGLITGGRRTRREAIVNAQPKCNPNLHYWTTQDEGAAIGLAWIPYFGPAAE
GIYIEGLMHNQDGLICGLRQLCNETTQALQLFLRATTELRTFSILNRKAIDFLLQRWGGTCHILGP
DCCIEPHDWTKNITDKIDQIIHDFVDKTLPIEGRHHHHHH
GP ΔTM-CC1-X-HIS
(SEQ ID NO: 18)
ATGGAGTTCTGGTTGTCTTGGGTGTTTCTCGTCGCTATTCTGAAGGGGGTGCAGT
GCGTGCCACTGGGAGTGATCCACAACAGCACTCTCCAAGTCTCCGACGTGGACAAGCTCG
TGTGCCGAGACAAGCTTTCCAGCACAAACCAGCTGCGGTCCGTGGGACTGAACCTGGAGG
GAAATGGCGTGGCCACCGATGTGCCGTCTGCAACCAAGAGATGGGGCTTCCGCTCGGGC
GTGCCCCCGAAAGTGGTCAACTACGAGGCCGGAGAATGGGCCGAAAACTGTTACAACCTG
GAGATCAAGAAGCCCGACGGTTCCGAATGCCTCCCTGCCGCCCCTGACGGAATCAGGGG
ATTCCCGAGATGCCGCTACGTGCACAAAGTGTCCGGGACTGGACCTTGTGCTGGAGATTT
CGCCTTCCACAAGGAGGGCGCCTTTTTCCTGTACGACCGGCTCGCATCCACCGTGATCTA
TAGAGGAACCACCTTCGCGGAAGGATGCGTCGCCTTCCTGATCCTGCCGCAAGCCAAGAA
GGACTTCTTTAGCTCCCATCCACTGCGCGAGCCCGTGAATGCCACCGAAGATCCCTCGTC
CGGCTATTACTCCACAACCATCCGGTACCAAGCCACCGGGTTCGGTACCAACGAGACTGA
GTACCTGTTCGAAGTCGACAACCTGACTTACGTGCAGCTCGAATCGCGGTTCACGCCACA
GTTCCTCCTGCAACTCAACGAAACTATCTACACTAGCGGGAAACGCTCGAACACCACCGG
GAAGCTGATTTGGAAGGTCAACCCTGAAATCGACACCACCATCGGAGAGTGGGCTTTTTG
GGAGACTAAGAAGAACCTCACCCGGAAGATCAGATCCGAGGAATTGTCCTTCACCGTGGT
CAACACCCACCACCAGGATACTGGAGAGGAGTCCGCATCAAGCGGAAAGCTCGGCCTGAT
TACTAACACTATCGCGGGGGTCGCTGGATTGATTACCGGTGGTAGAAGGACCAGGAGAGA
GGCCATTGTGAACGCGCAGCCGAAGTGCAACCCTAATCTGCATTACTGGACCACTCAGGA
CGAGGGCGCCGCCATCGGCCTGGCCTGGATTCCGTACTTCGGCCCGGCCGCGGAAGGC
ATATACATTGAGGGACTTATGCACAATCAGGATGGACTGATTTGTGGTCTGCGCCAGCTGT
GCAACGAAACCACCCAGGCGCTGCAGCTGTTCCTCCGGGCAACCACCGAACTTCGCACTT
TCTCCATCCTGAACCGGAAGGCCATTGACTTCCTCTTGCAACGCTGGGGAGGAACTTGCC
ACATCCTGGGTCCTGATTGCTGCATCGAACCGCATGACTGGACAAAGAACATCACCGACAA
AATCGACCAGATCATCCACGATTTCGTGGACAAGACCCTGCCTATCGAGGGCCGGCACCA
TCACCACCACCATTGATAA
GP ΔTM-CC2-X-HIS protein
(SEQ ID NO: 19)
MEFWLSWVFLVAILKGVQCVPLGVIHNSTLQVSDVDKLVCRDKLSSTNQLRSVGLNL
EGNGVATDVPSATKRWGFRSGVPPKVVNYEAGEWAENCYNLEIKKPDGSECLPAAPDGIRGF
PRCRYVHKVSGTGPCAGDFAFHKEGAFFLYDRLASTVIYRGTTFAEGVVAFLILPQAKKDFFSS
HPLREPVNATEDPSSGYYSTTIRYQATGFGTNETEYLFEVDNLTYVQLESRFTPQFLLQLNETIY
TSGKRSNTTGKLIWKVNPEIDTTIGEWAFCETKKNLTRKIRSEELSFTVVNTHHQDTGEESASS
GKLGLITNTIAGVAGLITGGRRTRREAIVNAQPKCCPNLHYWTTQDEGAAIGLAWIPYFGPAAE
GIYIEGLMHNQDGLICGLRQLANETTQALQLFLRATTELRTFSILNRKAIDFLLQRWGGTCHILGP
DCCIEPHDWTKNITDKIDQIIHDFVDKTLPIEGRHHHHHH
GP ΔTM-CC2-X-HIS DNA
(SEQ ID NO: 20)
ATGGAGTTCTGGTTGTCTTGGGTGTTTCTCGTCGCTATTCTGAAGGGGGTGCAGT
GCGTGCCACTGGGAGTGATCCACAACAGCACTCTCCAAGTCTCCGACGTGGACAAGCTCG
TGTGCCGAGACAAGCTTTCCAGCACAAACCAGCTGCGGTCCGTGGGACTGAACCTGGAGG
GAAATGGCGTGGCCACCGATGTGCCGTCTGCAACCAAGAGATGGGGCTTCCGCTCGGGC
GTGCCCCCGAAAGTGGTCAACTACGAGGCCGGAGAATGGGCCGAAAACTGTTACAACCTG
GAGATCAAGAAGCCCGACGGTTCCGAATGCCTCCCTGCCGCCCCTGACGGAATCAGGGG
ATTCCCGAGATGCCGCTACGTGCACAAAGTGTCCGGGACTGGACCTTGTGCTGGAGATTT
CGCCTTCCACAAGGAGGGCGCCTTTTTCCTGTACGACCGGCTCGCATCCACCGTGATCTA
TAGAGGAACCACCTTCGCGGAAGGAGTGGTCGCCTTCCTGATCCTGCCGCAAGCCAAGAA
GGACTTCTTTAGCTCCCATCCACTGCGCGAGCCCGTGAATGCCACCGAAGATCCCTCGTC
CGGCTATTACTCCACAACCATCCGGTACCAAGCCACCGGGTTCGGTACCAACGAGACTGA
GTACCTGTTCGAAGTCGACAACCTGACTTACGTGCAGCTCGAATCGCGGTTCACGCCACA
GTTCCTCCTGCAACTCAACGAAACTATCTACACTAGCGGGAAACGCTCGAACACCACCGG
GAAGCTGATTTGGAAGGTCAACCCTGAAATCGACACCACCATCGGAGAGTGGGCTTTTTG
CGAGACTAAGAAGAACCTCACCCGGAAGATCAGATCCGAGGAATTGTCCTTCACCGTGGT
CAACACCCACCACCAGGATACTGGAGAGGAGTCCGCATCAAGCGGAAAGCTCGGCCTGAT
TACTAACACTATCGCGGGGGTCGCTGGATTGATTACCGGTGGTAGAAGGACCAGGAGAGA
GGCCATTGTGAACGCGCAGCCGAAGTGCTGCCCTAATCTGCATTACTGGACCACTCAGGA
CGAGGGCGCCGCCATCGGCCTGGCCTGGATTCCGTACTTCGGCCCGGCCGCGGAAGGC
ATATACATTGAGGGACTTATGCACAATCAGGATGGACTGATTTGTGGTCTGCGCCAGCTGG
CCAACGAAACCACCCAGGCGCTGCAGCTGTTCCTCCGGGCAACCACCGAACTTCGCACTT
TCTCCATCCTGAACCGGAAGGCCATTGACTTCCTCTTGCAACGCTGGGGAGGAACTTGCC
ACATCCTGGGTCCTGATTGCTGCATCGAACCGCATGACTGGACAAAGAACATCACCGACAA
AATCGACCAGATCATCCACGATTTCGTGGACAAGACCCTGCCTATCGAGGGCCGGCACCA
TCACCACCACCATTGATAA
GP ΔTM-CC3-X-HIS protein
(SEQ ID NO: 21)
MEFWLSWVFLVAILKGVQCVPLGVIHNSTLQVSDVDKLVCRDKLSSTNQLRSVGLNL
EGNGVATDVPSATKRWGFRSGVPPKVVNYECGEWAENCYNLEIKKPDGSECLPAAPDGIRGF
PRCRYVHKVSGTGPCAGDFAFHKEGAFFLYDRLASTVIYRGTTFAEGVVAFLILPQAKKDFFSS
HPLREPVNATEDPSSGYYSTTIRYQATGFGTNETEYLFEVDNLTYVQLESRFTPQFLLQLNETIY
TSGKRSNTTGKLIWKVNPEIDTTIGEWAFWETKKNLTRKIRSEELSFTVVNTHHQDTGEESASS
GKLGLITNTIAGVAGLITGGRRTRREAIVNAQPKCNPNLHYWCTQDEGAAIGLAWIPYFGPAAE
GIYIEGLMHNQDGLICGLRQLANETTQALQLFLRATTELRTFSILNRKAIDFLLQRWGGTCHILGP
DCCIEPHDWTKNITDKIDQIIHDFVDKTLPIEGRHHHHHH
GP ΔTM-CC3-X-HIS DNA
(SEQ ID NO: 22)
ATGGAGTTCTGGTTGTCTTGGGTGTTTCTCGTCGCTATTCTGAAGGGGGTGCAGT
GCGTGCCACTGGGAGTGATCCACAACAGCACTCTCCAAGTCTCCGACGTGGACAAGCTCG
TGTGCCGAGACAAGCTTTCCAGCACAAACCAGCTGCGGTCCGTGGGACTGAACCTGGAGG
GAAATGGCGTGGCCACCGATGTGCCGTCTGCAACCAAGAGATGGGGCTTCCGCTCGGGC
GTGCCCCCGAAAGTGGTCAACTACGAGTGCGGAGAATGGGCCGAAAACTGTTACAACCTG
GAGATCAAGAAGCCCGACGGTTCCGAATGCCTCCCTGCCGCCCCTGACGGAATCAGGGG
ATTCCCGAGATGCCGCTACGTGCACAAAGTGTCCGGGACTGGACCTTGTGCTGGAGATTT
CGCCTTCCACAAGGAGGGCGCCTTTTTCCTGTACGACCGGCTCGCATCCACCGTGATCTA
TAGAGGAACCACCTTCGCGGAAGGAGTGGTCGCCTTCCTGATCCTGCCGCAAGCCAAGAA
GGACTTCTTTAGCTCCCATCCACTGCGCGAGCCCGTGAATGCCACCGAAGATCCCTCGTC
CGGCTATTACTCCACAACCATCCGGTACCAAGCCACCGGGTTCGGTACCAACGAGACTGA
GTACCTGTTCGAAGTCGACAACCTGACTTACGTGCAGCTCGAATCGCGGTTCACGCCACA
GTTCCTCCTGCAACTCAACGAAACTATCTACACTAGCGGGAAACGCTCGAACACCACCGG
GAAGCTGATTTGGAAGGTCAACCCTGAAATCGACACCACCATCGGAGAGTGGGCTTTTTG
GGAGACTAAGAAGAACCTCACCCGGAAGATCAGATCCGAGGAATTGTCCTTCACCGTGGT
CAACACCCACCACCAGGATACTGGAGAGGAGTCCGCATCAAGCGGAAAGCTCGGCCTGAT
TACTAACACTATCGCGGGGGTCGCTGGATTGATTACCGGTGGTAGAAGGACCAGGAGAGA
GGCCATTGTGAACGCGCAGCCGAAGTGCAACCCTAATCTGCATTACTGGTGCACTCAGGA
CGAGGGCGCCGCCATCGGCCTGGCCTGGATTCCGTACTTCGGCCCGGCCGCGGAAGGC
ATATACATTGAGGGACTTATGCACAATCAGGATGGACTGATTTGTGGTCTGCGCCAGCTGG
CCAACGAAACCACCCAGGCGCTGCAGCTGTTCCTCCGGGCAACCACCGAACTTCGCACTT
TCTCCATCCTGAACCGGAAGGCCATTGACTTCCTCTTGCAACGCTGGGGAGGAACTTGCC
ACATCCTGGGTCCTGATTGCTGCATCGAACCGCATGACTGGACAAAGAACATCACCGACAA
AATCGACCAGATCATCCACGATTTCGTGGACAAGACCCTGCCTATCGAGGGCCGGCACCA
TCACCACCACCATTGATAA
GP ΔTM-CC4-X-HIS protein
(SEQ ID NO: 23)
MEFWLSWVFLVAILKGVQCVPLGVIHNSTLQVSDVDKLVCRDKLSSTNQLRSVGLNL
EGNGVATDVPSATKRWGFRSGVPPKVVNYECGEWAENCYNLEIKKPDGSECLPAAPDGIRGF
PRCRYVHKVSGTGPCAGDFAFHKEGAFFLYDRLASTVIYRGTTFAEGVVAFLILPQAKKDFCSS
HPLREPVNATEDPSSGYYSTTIRYQATGFGTNETEYLFEVDNLTYVQLESRFTPQFLLQLNETIY
TSGKRSNTTGKLIWKVNPEIDTTIGEWAFWETKKNLTRKIRSEELSFTVVNTHHQDTGEESASS
GKLGLITNTIAGVAGLITGGRRTRREAIVNAQPKCNPNLHYWTTQDEGAAIGLAWIPYFGPAAE
GIYIEGLMHNQDGLICGLRQLANETTQALQLFLRATTELRTFSILNRKAIDFLLQRWGGTCHILGP
DCCIEPHDWTKNITDKIDQIIHDFVDKTLPIEGRHHHHHH
GP ΔTM-CC4-X-HIS DNA
(SEQ ID NO: 24)
ATGGAGTTCTGGTTGTCTTGGGTGTTTCTCGTCGCTATTCTGAAGGGGGTGCAGT
GCGTGCCACTGGGAGTGATCCACAACAGCACTCTCCAAGTCTCCGACGTGGACAAGCTCG
TGTGCCGAGACAAGCTTTCCAGCACAAACCAGCTGCGGTCCGTGGGACTGAACCTGGAGG
GAAATGGCGTGGCCACCGATGTGCCGTCTGCAACCAAGAGATGGGGCTTCCGCTCGGGC
GTGCCCCCGAAAGTGGTCAACTACGAGTGCGGAGAATGGGCCGAAAACTGTTACAACCTG
GAGATCAAGAAGCCCGACGGTTCCGAATGCCTCCCTGCCGCCCCTGACGGAATCAGGGG
ATTCCCGAGATGCCGCTACGTGCACAAAGTGTCCGGGACTGGACCTTGTGCTGGAGATTT
CGCCTTCCACAAGGAGGGCGCCTTTTTCCTGTACGACCGGCTCGCATCCACCGTGATCTA
TAGAGGAACCACCTTCGCGGAAGGAGTGGTCGCCTTCCTGATCCTGCCGCAAGCCAAGAA
GGACTTCTGCAGCTCCCATCCACTGCGCGAGCCCGTGAATGCCACCGAAGATCCCTCGTC
CGGCTATTACTCCACAACCATCCGGTACCAAGCCACCGGGTTCGGTACCAACGAGACTGA
GTACCTGTTCGAAGTCGACAACCTGACTTACGTGCAGCTCGAATCGCGGTTCACGCCACA
GTTCCTCCTGCAACTCAACGAAACTATCTACACTAGCGGGAAACGCTCGAACACCACCGG
GAAGCTGATTTGGAAGGTCAACCCTGAAATCGACACCACCATCGGAGAGTGGGCTTTTTG
GGAGACTAAGAAGAACCTCACCCGGAAGATCAGATCCGAGGAATTGTCCTTCACCGTGGT
CAACACCCACCACCAGGATACTGGAGAGGAGTCCGCATCAAGCGGAAAGCTCGGCCTGAT
TACTAACACTATCGCGGGGGTCGCTGGATTGATTACCGGTGGTAGAAGGACCAGGAGAGA
GGCCATTGTGAACGCGCAGCCGAAGTGCAACCCTAATCTGCATTACTGGACCACTCAGGA
CGAGGGCGCCGCCATCGGCCTGGCCTGGATTCCGTACTTCGGCCCGGCCGCGGAAGGC
ATATACATTGAGGGACTTATGCACAATCAGGATGGACTGATTTGTGGTCTGCGCCAGCTGG
CCAACGAAACCACCCAGGCGCTGCAGCTGTTCCTCCGGGCAACCACCGAACTTCGCACTT
TCTCCATCCTGAACCGGAAGGCCATTGACTTCCTCTTGCAACGCTGGGGAGGAACTTGCC
ACATCCTGGGTCCTGATTGCTGCATCGAACCGCATGACTGGACAAAGAACATCACCGACAA
AATCGACCAGATCATCCACGATTTCGTGGACAAGACCCTGCCTATCGAGGGCCGGCACCA
TCACCACCACCATTGATAA
GP ΔTM-CC5-X-HIS protein
(SEQ ID NO: 25)
MEFWLSWVFLVAILKGVQCVPLGVIHNSTLQVSDVDKLVCRDKLSSTNQLRSVGLN
LEGNGVATDVPSATKRWGFRSGVPPKVCNYEAGEWAENCYNLEIKKPDGSECLPAAPDGIRG
FPRCRYVHKVSGTGPCAGDFAFHKEGAFFLYDRLASTVIYRGTTFAEGVVAFLILPQAKKDFFS
SHPLREPVNATEDPSSGYYSTTIRYQATGFGTNETEYLFEVDNLTYVQLESRFTPQFLLQLNETI
YTSGKRSNTTGKLIWKVNPEIDTTIGEWAFWETKKNLTRKIRSEELSFTVVNTHHQDTGEESAS
SGKLGLITNTIAGVAGLITGGRRTRREAIVNAQPKCNPNLHYWTTQDEGAAIGLAWIPYFGPAAE
GIYIEGLMHNQDGLICGLRQLANETTQALQLFLRATTELRCFSILNRKAIDFLLQRWGGTCHILGP
DCCIEPHDWTKNITDKIDQIIHDFVDKTLPIEGRHHHHHH
GP ΔTM-CC5-X-HIS DNA
(SEQ ID NO: 26)
ATGGAGTTCTGGTTGTCTTGGGTGTTTCTCGTCGCTATTCTGAAGGGGGTGCAG
TGCGTGCCACTGGGAGTGATCCACAACAGCACTCTCCAAGTCTCCGACGTGGACAAGCTC
GTGTGCCGAGACAAGCTTTCCAGCACAAACCAGCTGCGGTCCGTGGGACTGAACCTGGAG
GGAAATGGCGTGGCCACCGATGTGCCGTCTGCAACCAAGAGATGGGGCTTCCGCTCGGG
CGTGCCCCCGAAAGTGTGTAACTACGAGGCCGGAGAATGGGCCGAAAACTGTTACAACCT
GGAGATCAAGAAGCCCGACGGTTCCGAATGCCTCCCTGCCGCCCCTGACGGAATCAGGG
GATTCCCGAGATGCCGCTACGTGCACAAAGTGTCCGGGACTGGACCTTGTGCTGGAGATT
TCGCCTTCCACAAGGAGGGCGCCTTTTTCCTGTACGACCGGCTCGCATCCACCGTGATCT
ATAGAGGAACCACCTTCGCGGAAGGAGTGGTCGCCTTCCTGATCCTGCCGCAAGCCAAGA
AGGACTTCTTTAGCTCCCATCCACTGCGCGAGCCCGTGAATGCCACCGAAGATCCCTCGT
CCGGCTATTACTCCACAACCATCCGGTACCAAGCCACCGGGTTCGGTACCAACGAGACTG
AGTACCTGTTCGAAGTCGACAACCTGACTTACGTGCAGCTCGAATCGCGGTTCACGCCAC
AGTTCCTCCTGCAACTCAACGAAACTATCTACACTAGCGGGAAACGCTCGAACACCACCGG
GAAGCTGATTTGGAAGGTCAACCCTGAAATCGACACCACCATCGGAGAGTGGGCTTTTTG
GGAGACTAAGAAGAACCTCACCCGGAAGATCAGATCCGAGGAATTGTCCTTCACCGTGGT
CAACACCCACCACCAGGATACTGGAGAGGAGTCCGCATCAAGCGGAAAGCTCGGCCTGAT
TACTAACACTATCGCGGGGGTCGCTGGATTGATTACCGGTGGTAGAAGGACCAGGAGAGA
GGCCATTGTGAACGCGCAGCCGAAGTGCAACCCTAATCTGCATTACTGGACCACTCAGGA
CGAGGGCGCCGCCATCGGCCTGGCCTGGATTCCGTACTTCGGCCCGGCCGCGGAAGGC
ATATACATTGAGGGACTTATGCACAATCAGGATGGACTGATTTGTGGTCTGCGCCAGCTGG
CCAACGAAACCACCCAGGCGCTGCAGCTGTTCCTCCGGGCAACCACCGAACTTCGCTGTT
TCTCCATCCTGAACCGGAAGGCCATTGACTTCCTCTTGCAACGCTGGGGAGGAACTTGCC
ACATCCTGGGTCCTGATTGCTGCATCGAACCGCATGACTGGACAAAGAACATCACCGACAA
AATCGACCAGATCATCCACGATTTCGTGGACAAGACCCTGCCTATCGAGGGCCGGCACCA
TCACCACCACCATTGATAA
GP ΔTM-CC6-X-HIS protein
(SEQ ID NO: 27)
MEFWLSWVFLVAILKGVQCVPLGVIHNSTLQVSDVDKLVCRDKLSSTNQCRSVGLN
LEGNGVATDVPSATKRWGFRSGVPPKVVNYEAGEWAENCYNLEIKKPDGSECLPAAPDGIRG
FPRCRYVHKVSGTGPCAGDFAFHKEGAFFLYDRLASTVIYRGTTFAEGVVAFLILPQAKKDFFS
SHPLREPVNATEDPSSGYYSTTIRYQATGFGTNETEYLFEVDNLTYVQLESRFTPQFLLQLNETI
YTSGKRSNTTGKLIWKVNPEIDTTIGEWAFWETKKNLTRKIRSEELSFTVVNTHHQDTGEESAS
SGKLGLITNTIAGVAGLITGGRRTRREAIVNAQPKCNPNLHYWTTQDEGAAIGLAWIPYFGPAAE
GIYIEGLMHNQDGLICGLRQLANETTQALQLFLRATTELRTFSICNRKAIDFLLQRWGGTCHILG
PDCCIEPHDWTKNITDKIDQIIHDFVDKTLPIEGRHHHHHH
GP ΔTM-CC6-X-HIS DNA
(SEQ ID NO: 28)
ATGGAGTTCTGGTTGTCTTGGGTGTTTCTCGTCGCTATTCTGAAGGGGGTGCAG
TGCGTGCCACTGGGAGTGATCCACAACAGCACTCTCCAAGTCTCCGACGTGGACAAGCTC
GTGTGCCGAGACAAGCTTTCCAGCACAAACCAGTGCCGGTCCGTGGGACTGAACCTGGAG
GGAAATGGCGTGGCCACCGATGTGCCGTCTGCAACCAAGAGATGGGGCTTCCGCTCGGG
CGTGCCCCCGAAAGTGGTCAACTACGAGGCCGGAGAATGGGCCGAAAACTGTTACAACCT
GGAGATCAAGAAGCCCGACGGTTCCGAATGCCTCCCTGCCGCCCCTGACGGAATCAGGG
GATTCCCGAGATGCCGCTACGTGCACAAAGTGTCCGGGACTGGACCTTGTGCTGGAGATT
TCGCCTTCCACAAGGAGGGCGCCTTTTTCCTGTACGACCGGCTCGCATCCACCGTGATCT
ATAGAGGAACCACCTTCGCGGAAGGAGTGGTCGCCTTCCTGATCCTGCCGCAAGCCAAGA
AGGACTTCTTTAGCTCCCATCCACTGCGCGAGCCCGTGAATGCCACCGAAGATCCCTCGT
CCGGCTATTACTCCACAACCATCCGGTACCAAGCCACCGGGTTCGGTACCAACGAGACTG
AGTACCTGTTCGAAGTCGACAACCTGACTTACGTGCAGCTCGAATCGCGGTTCACGCCAC
AGTTCCTCCTGCAACTCAACGAAACTATCTACACTAGCGGGAAACGCTCGAACACCACCGG
GAAGCTGATTTGGAAGGTCAACCCTGAAATCGACACCACCATCGGAGAGTGGGCTTTTTG
GGAGACTAAGAAGAACCTCACCCGGAAGATCAGATCCGAGGAATTGTCCTTCACCGTGGT
CAACACCCACCACCAGGATACTGGAGAGGAGTCCGCATCAAGCGGAAAGCTCGGCCTGAT
TACTAACACTATCGCGGGGGTCGCTGGATTGATTACCGGTGGTAGAAGGACCAGGAGAGA
GGCCATTGTGAACGCGCAGCCGAAGTGCAACCCTAATCTGCATTACTGGACCACTCAGGA
CGAGGGCGCCGCCATCGGCCTGGCCTGGATTCCGTACTTCGGCCCGGCCGCGGAAGGC
ATATACATTGAGGGACTTATGCACAATCAGGATGGACTGATTTGTGGTCTGCGCCAGCTGG
CCAACGAAACCACCCAGGCGCTGCAGCTGTTCCTCCGGGCAACCACCGAACTTCGCACTT
TCTCCATCTGCAACCGGAAGGCCATTGACTTCCTCTTGCAACGCTGGGGAGGAACTTGCC
ACATCCTGGGTCCTGATTGCTGCATCGAACCGCATGACTGGACAAAGAACATCACCGACAA
AATCGACCAGATCATCCACGATTTCGTGGACAAGACCCTGCCTATCGAGGGCCGGCACCA
TCACCACCACCATTGATAA
GP ΔTM-CC7-X-HIS protein
(SEQ ID NO: 29)
MEFWLSWVFLVAILKGVQCVPLGVIHNCTLQVSDVDKLVCRDKLSSTNQLRSVGLN
LEGNGVATDVPSATKRWGFRSGVPPKVVNYEAGEWAENCYNLEIKKPDGSECLPAAPDGIRG
FPRCRYVHKVSGTGPCAGDFAFHKEGAFFLYDRLASTVIYRGTTFAEGVVAFLILPQAKKDFFS
SHPLREPVNATEDPSSGYYSTTIRYQATGFGTNETEYLFEVDNLTYVQLESRFTPQFLLQLNETI
YTSGKRSNTTGKLIWKVNPEIDTTIGEWAFWETKKNLTRKIRSEELSFTVVNTHHQDTGEESAS
SGKLGLITNTIAGVAGLITGGRRTRREAIVNAQPKCNPNLHYWTTQDEGAAIGLAWIPYFGPAAE
GIYIEGLMHNQCGLICGLRQLANETTQALQLFLRATTELRTFSILNRKAIDFLLQRWGGTCHILGP
DCCIEPHDWTKNITDKIDQIIHDFVDKTLPIEGRHHHHHH
GP ΔTM-CC7-X-HIS DNA
(SEQ ID NO: 30)
ATGGAGTTCTGGTTGTCTTGGGTGTTTCTCGTCGCTATTCTGAAGGGGGTGCAG
TGCGTGCCACTGGGAGTGATCCACAACTGCACTCTCCAAGTCTCCGACGTGGACAAGCTC
GTGTGCCGAGACAAGCTTTCCAGCACAAACCAGCTGCGGTCCGTGGGACTGAACCTGGAG
GGAAATGGCGTGGCCACCGATGTGCCGTCTGCAACCAAGAGATGGGGCTTCCGCTCGGG
CGTGCCCCCGAAAGTGGTCAACTACGAGGCCGGAGAATGGGCCGAAAACTGTTACAACCT
GGAGATCAAGAAGCCCGACGGTTCCGAATGCCTCCCTGCCGCCCCTGACGGAATCAGGG
GATTCCCGAGATGCCGCTACGTGCACAAAGTGTCCGGGACTGGACCTTGTGCTGGAGATT
TCGCCTTCCACAAGGAGGGCGCCTTTTTCCTGTACGACCGGCTCGCATCCACCGTGATCT
ATAGAGGAACCACCTTCGCGGAAGGAGTGGTCGCCTTCCTGATCCTGCCGCAAGCCAAGA
AGGACTTCTTTAGCTCCCATCCACTGCGCGAGCCCGTGAATGCCACCGAAGATCCCTCGT
CCGGCTATTACTCCACAACCATCCGGTACCAAGCCACCGGGTTCGGTACCAACGAGACTG
AGTACCTGTTCGAAGTCGACAACCTGACTTACGTGCAGCTCGAATCGCGGTTCACGCCAC
AGTTCCTCCTGCAACTCAACGAAACTATCTACACTAGCGGGAAACGCTCGAACACCACCGG
GAAGCTGATTTGGAAGGTCAACCCTGAAATCGACACCACCATCGGAGAGTGGGCTTTTTG
GGAGACTAAGAAGAACCTCACCCGGAAGATCAGATCCGAGGAATTGTCCTTCACCGTGGT
CAACACCCACCACCAGGATACTGGAGAGGAGTCCGCATCAAGCGGAAAGCTCGGCCTGAT
TACTAACACTATCGCGGGGGTCGCTGGATTGATTACCGGTGGTAGAAGGACCAGGAGAGA
GGCCATTGTGAACGCGCAGCCGAAGTGCAACCCTAATCTGCATTACTGGACCACTCAGGA
CGAGGGCGCCGCCATCGGCCTGGCCTGGATTCCGTACTTCGGCCCGGCCGCGGAAGGC
ATATACATTGAGGGACTTATGCACAATCAGTGCGGACTGATTTGTGGTCTGCGCCAGCTGG
CCAACGAAACCACCCAGGCGCTGCAGCTGTTCCTCCGGGCAACCACCGAACTTCGCACTT
TCTCCATCCTGAACCGGAAGGCCATTGACTTCCTCTTGCAACGCTGGGGAGGAACTTGCC
ACATCCTGGGTCCTGATTGCTGCATCGAACCGCATGACTGGACAAAGAACATCACCGACAA
AATCGACCAGATCATCCACGATTTCGTGGACAAGACCCTGCCTATCGAGGGCCGGCACCA
TCACCACCACCATTGATAA
GP ΔTM-CC8-X-HIS protein
(SEQ ID NO: 31)
MEFWLSWVFLVAILKGVQCVPLGVIHNSTLQVSDVDKLVCRDKLSSTNQLRSVGLN
LEGNGVATDVPSATKRWGFRSGVPPKVCNYEAGEWAENCYNLEIKKPDGSECLPAAPDGIRG
FPRCRYVHKVSGTGPCAGDFAFHKEGAFFLYDRLASTVIYRGTTFAEGVVAFLILPQAKKDFFS
SHPLREPVNATEDPSSGYYSTTIRYQATGFGTNETEYLFEVDNLTYVQLESRFTPQFLLQLNETI
YTSGKRSNTTGKLIWKVNPEIDTTIGEWAFCETKKNLTRKIRSEELSFTVVNTHHQDTGEESAS
SGKLGLITNTIAGVAGLITGGRRTRREAIVNAQPKCCPNLHYWTTQDEGAAIGLAWIPYFGPAAE
GIYIEGLMHNQDGLICGLRQLANETTQALQLFLRATTELRCFSILNRKAIDFLLQRWGGTCHILGP
DCCIEPHDWTKNITDKIDQIIHDFVDKTLPIEGRHHHHHH
GP ΔTM-CC8-X-HIS DNA
(SEQ ID NO: 32)
ATGGAGTTCTGGTTGTCTTGGGTGTTTCTCGTCGCTATTCTGAAGGGGGTGCAG
TGCGTGCCACTGGGAGTGATCCACAACAGCACTCTCCAAGTCTCCGACGTGGACAAGCTC
GTGTGCCGAGACAAGCTTTCCAGCACAAACCAGCTGCGGTCCGTGGGACTGAACCTGGAG
GGAAATGGCGTGGCCACCGATGTGCCGTCTGCAACCAAGAGATGGGGCTTCCGCTCGGG
CGTGCCCCCGAAAGTGTGCAACTACGAGGCCGGAGAATGGGCCGAAAACTGTTACAACCT
GGAGATCAAGAAGCCCGACGGTTCCGAATGCCTCCCTGCCGCCCCTGACGGAATCAGGG
GATTCCCGAGATGCCGCTACGTGCACAAAGTGTCCGGGACTGGACCTTGTGCTGGAGATT
TCGCCTTCCACAAGGAGGGCGCCTTTTTCCTGTACGACCGGCTCGCATCCACCGTGATCT
ATAGAGGAACCACCTTCGCGGAAGGAGTGGTCGCCTTCCTGATCCTGCCGCAAGCCAAGA
AGGACTTCTTTAGCTCCCATCCACTGCGCGAGCCCGTGAATGCCACCGAAGATCCCTCGT
CCGGCTATTACTCCACAACCATCCGGTACCAAGCCACCGGGTTCGGTACCAACGAGACTG
AGTACCTGTTCGAAGTCGACAACCTGACTTACGTGCAGCTCGAATCGCGGTTCACGCCAC
AGTTCCTCCTGCAACTCAACGAAACTATCTACACTAGCGGGAAACGCTCGAACACCACCGG
GAAGCTGATTTGGAAGGTCAACCCTGAAATCGACACCACCATCGGAGAGTGGGCTTTTTG
CGAGACTAAGAAGAACCTCACCCGGAAGATCAGATCCGAGGAATTGTCCTTCACCGTGGT
CAACACCCACCACCAGGATACTGGAGAGGAGTCCGCATCAAGCGGAAAGCTCGGCCTGAT
TACTAACACTATCGCGGGGGTCGCTGGATTGATTACCGGTGGTAGAAGGACCAGGAGAGA
GGCCATTGTGAACGCGCAGCCGAAGTGCTGCCCTAATCTGCATTACTGGACCACTCAGGA
CGAGGGCGCCGCCATCGGCCTGGCCTGGATTCCGTACTTCGGCCCGGCCGCGGAAGGC
ATATACATTGAGGGACTTATGCACAATCAGGATGGACTGATTTGTGGTCTGCGCCAGCTGG
CCAACGAAACCACCCAGGCGCTGCAGCTGTTCCTCCGGGCAACCACCGAACTTCGCTGTT
TCTCCATCCTGAACCGGAAGGCCATTGACTTCCTCTTGCAACGCTGGGGAGGAACTTGCC
ACATCCTGGGTCCTGATTGCTGCATCGAACCGCATGACTGGACAAAGAACATCACCGACAA
AATCGACCAGATCATCCACGATTTCGTGGACAAGACCCTGCCTATCGAGGGCCGGCACCA
TCACCACCACCATTGATAA
GP ΔTM-CC9-X-HIS protein
(SEQ ID NO: 33)
MEFWLSWVFLVAILKGVQCVPLGVIHNCTLQVSDVDKLVCRDKLSSTNQLRSVGLN
LEGNGVATDVPSATKRWGFRSGVPPKVVNYECGEWAENCYNLEIKKPDGSECLPAAPDGIRG
FPRCRYVHKVSGTGPCAGDFAFHKEGAFFLYDRLASTVIYRGTTFAEGVVAFLILPQAKKDFFS
SHPLREPVNATEDPSSGYYSTTIRYQATGFGTNETEYLFEVDNLTYVQLESRFTPQFLLQLNETI
YTSGKRSNTTGKLIWKVNPEIDTTIGEWAFWETKKNLTRKIRSEELSFTVVNTHHQDTGEESAS
SGKLGLITNTIAGVAGLITGGRRTRREAIVNAQPKCNPNLHYWCTQDEGAAIGLAWIPYFGPAA
EGIYIEGLMHNQCGLICGLRQLANETTQALQLFLRATTELRTFSILNRKAIDFLLQRWGGTCHILG
PDCCIEPHDWTKNITDKIDQIIHDFVDKTLPIEGRHHHHHH
GP ΔTM-CC9-X-HIS DNA
(SEQ ID NO: 34)
ATGGAGTTCTGGTTGTCTTGGGTGTTTCTCGTCGCTATTCTGAAGGGGGTGCAG
TGCGTGCCACTGGGAGTGATCCACAACTGCACTCTCCAAGTCTCCGACGTGGACAAGCTC
GTGTGCCGAGACAAGCTTTCCAGCACAAACCAGCTGCGGTCCGTGGGACTGAACCTGGAG
GGAAATGGCGTGGCCACCGATGTGCCGTCTGCAACCAAGAGATGGGGCTTCCGCTCGGG
CGTGCCCCCGAAAGTGGTCAACTACGAGTGCGGAGAATGGGCCGAAAACTGTTACAACCT
GGAGATCAAGAAGCCCGACGGTTCCGAATGCCTCCCTGCCGCCCCTGACGGAATCAGGG
GATTCCCGAGATGCCGCTACGTGCACAAAGTGTCCGGGACTGGACCTTGTGCTGGAGATT
TCGCCTTCCACAAGGAGGGCGCCTTTTTCCTGTACGACCGGCTCGCATCCACCGTGATCT
ATAGAGGAACCACCTTCGCGGAAGGAGTGGTCGCCTTCCTGATCCTGCCGCAAGCCAAGA
AGGACTTCTTTAGCTCCCATCCACTGCGCGAGCCCGTGAATGCCACCGAAGATCCCTCGT
CCGGCTATTACTCCACAACCATCCGGTACCAAGCCACCGGGTTCGGTACCAACGAGACTG
AGTACCTGTTCGAAGTCGACAACCTGACTTACGTGCAGCTCGAATCGCGGTTCACGCCAC
AGTTCCTCCTGCAACTCAACGAAACTATCTACACTAGCGGGAAACGCTCGAACACCACCGG
GAAGCTGATTTGGAAGGTCAACCCTGAAATCGACACCACCATCGGAGAGTGGGCTTTTTG
GGAGACTAAGAAGAACCTCACCCGGAAGATCAGATCCGAGGAATTGTCCTTCACCGTGGT
CAACACCCACCACCAGGATACTGGAGAGGAGTCCGCATCAAGCGGAAAGCTCGGCCTGAT
TACTAACACTATCGCGGGGGTCGCTGGATTGATTACCGGTGGTAGAAGGACCAGGAGAGA
GGCCATTGTGAACGCGCAGCCGAAGTGCAACCCTAATCTGCATTACTGGTGTACTCAGGA
CGAGGGCGCCGCCATCGGCCTGGCCTGGATTCCGTACTTCGGCCCGGCCGCGGAAGGC
ATATACATTGAGGGACTTATGCACAATCAGTGTGGACTGATTTGTGGTCTGCGCCAGCTGG
CCAACGAAACCACCCAGGCGCTGCAGCTGTTCCTCCGGGCAACCACCGAACTTCGCACTT
TCTCCATCCTGAACCGGAAGGCCATTGACTTCCTCTTGCAACGCTGGGGAGGAACTTGCC
ACATCCTGGGTCCTGATTGCTGCATCGAACCGCATGACTGGACAAAGAACATCACCGACAA
AATCGACCAGATCATCCACGATTTCGTGGACAAGACCCTGCCTATCGAGGGCCGGCACCA
TCACCACCACCATTGATAA
GP ΔTM-CC10-X-HIS protein
(SEQ ID NO: 35)
MEFWLSWVFLVAILKGVQCVPLGVIHNSTLQVSDVDKLVCRDKLSSTNQLRSVGLN
LEGNGVATDVPSATKRWGFRSGVPPKVVNYEAGEWAENCYNLEIKKPDGSECLPAAPDGIRG
FPRCRYVHKVSGTGPCAGDFAFHKEGAFFLYDRLASTVIYRGTTFAEGVVAFLILPQAKKDFFS
SHPLREPVNATEDPSSGYYSTTIRYQATGFGTNETEYLFEVDNLCYVQLESRFTPQFLLQLNETI
YTSGKRCNTTGKLIWKVNPEIDTTIGEWAFWETKKNLTRKIRSEELSFTVVNTHHQDTGEESAS
SGKLGLITNTIAGVAGLITGGRRTRREAIVNAQPKCNPNLHYWTTQDEGAAIGLAWIPYFGPAAE
GIYIEGLMHNQDGLICGLRQLANETTQALQLFLRATTELRTFSILNRKAIDFLLQRWGGTCHILGP
DCCIEPHDWTKNITDKIDQIIHDFVDKTLPIEGRHHHHHH
GP ΔTM-CC10-X-HIS DNA
(SEQ ID NO: 36)
ATGGAGTTCTGGTTGTCTTGGGTGTTTCTCGTCGCTATTCTGAAGGGGGTGCAG
TGCGTGCCACTGGGAGTGATCCACAACAGCACTCTCCAAGTCTCCGACGTGGACAAGCTC
GTGTGCCGAGACAAGCTTTCCAGCACAAACCAGCTGCGGTCCGTGGGACTGAACCTGGAG
GGAAATGGCGTGGCCACCGATGTGCCGTCTGCAACCAAGAGATGGGGCTTCCGCTCGGG
CGTGCCCCCGAAAGTGGTCAACTACGAGGCCGGAGAATGGGCCGAAAACTGTTACAACCT
GGAGATCAAGAAGCCCGACGGTTCCGAATGCCTCCCTGCCGCCCCTGACGGAATCAGGG
GATTCCCGAGATGCCGCTACGTGCACAAAGTGTCCGGGACTGGACCTTGTGCTGGAGATT
TCGCCTTCCACAAGGAGGGCGCCTTTTTCCTGTACGACCGGCTCGCATCCACCGTGATCT
ATAGAGGAACCACCTTCGCGGAAGGAGTGGTCGCCTTCCTGATCCTGCCGCAAGCCAAGA
AGGACTTCTTTAGCTCCCATCCACTGCGCGAGCCCGTGAATGCCACCGAAGATCCCTCGT
CCGGCTATTACTCCACAACCATCCGGTACCAAGCCACCGGGTTCGGTACCAACGAGACTG
AGTACCTGTTCGAAGTCGACAACCTGTGCTACGTGCAGCTCGAATCGCGGTTCACGCCAC
AGTTCCTCCTGCAACTCAACGAAACTATCTACACTAGCGGGAAACGCTGCAACACCACCGG
GAAGCTGATTTGGAAGGTCAACCCTGAAATCGACACCACCATCGGAGAGTGGGCTTTTTG
GGAGACTAAGAAGAACCTCACCCGGAAGATCAGATCCGAGGAATTGTCCTTCACCGTGGT
CAACACCCACCACCAGGATACTGGAGAGGAGTCCGCATCAAGCGGAAAGCTCGGCCTGAT
TACTAACACTATCGCGGGGGTCGCTGGATTGATTACCGGTGGTAGAAGGACCAGGAGAGA
GGCCATTGTGAACGCGCAGCCGAAGTGCAACCCTAATCTGCATTACTGGACCACTCAGGA
CGAGGGCGCCGCCATCGGCCTGGCCTGGATTCCGTACTTCGGCCCGGCCGCGGAAGGC
ATATACATTGAGGGACTTATGCACAATCAGGATGGACTGATTTGTGGTCTGCGCCAGCTGG
CCAACGAAACCACCCAGGCGCTGCAGCTGTTCCTCCGGGCAACCACCGAACTTCGCACTT
TCTCCATCCTGAACCGGAAGGCCATTGACTTCCTCTTGCAACGCTGGGGAGGAACTTGCC
ACATCCTGGGTCCTGATTGCTGCATCGAACCGCATGACTGGACAAAGAACATCACCGACAA
AATCGACCAGATCATCCACGATTTCGTGGACAAGACCCTGCCTATCGAGGGCCGGCACCA
TCACCACCACCATTGATAA
GP ΔTM-ΔMUC-T4-X-HIS protein
(SEQ ID NO: 37)
MEFWLSWVFLVAILKGVQCVPLGVIHNSTLQVSDVDKLVCRDKLSSTNQLRSVGLN
LEGNGVATDVPSATKRWGFRSGVPPKVVNYEAGEWAENCYNLEIKKPDGSECLPAAPDGIRG
FPRCRYVHKVSGTGPCAGDFAFHKEGAFFLYDRLASTVIYRGTTFAEGVVAFLILPQAKKDFFS
SHPLREPVNATEDPSSGYYSTTIRYQATGFGTNETEYLFEVDNLTYVQLESRFTPQFLLQLNETI
YTSGKRSNTTGKLIWKVNPEIDTTIGEWAFWETKKNLTRKIRSEELSFTVVNTHHQDTGEESAS
SGKLGLITNTIAGVAGLITGGRRTRREAIVNAQPKCNPNLHYWTTQDEGAAIGLAWIPYFGPAAE
GIYIEGLMHNQDGLICGLRQLANETTQALQLFLRATTELRTFSILNRKAIDFLLQRWGGTCHILGP
DCCIEPHDWTKNITDKIDQIIHDFVDKTLPGSGYIPEAPRDGQAYVRKDGEWVLLSTFLGTIEGR
HHHHHH
GP ΔTM-ΔMUC-T4-X-HIS DNA
(SEQ ID NO: 38)
ATGGAGTTCTGGTTGTCTTGGGTGTTTCTCGTCGCTATTCTGAAGGGGGTGCAG
TGCGTGCCACTGGGAGTGATCCACAACAGCACTCTCCAAGTCTCCGACGTGGACAAGCTC
GTGTGCCGAGACAAGCTTTCCAGCACAAACCAGCTGCGGTCCGTGGGACTGAACCTGGAG
GGAAATGGCGTGGCCACCGATGTGCCGTCTGCAACCAAGAGATGGGGCTTCCGCTCGGG
CGTGCCCCCGAAAGTGGTCAACTACGAGGCCGGAGAATGGGCCGAAAACTGTTACAACCT
GGAGATCAAGAAGCCCGACGGTTCCGAATGCCTCCCTGCCGCCCCTGACGGAATCAGGG
GATTCCCGAGATGCCGCTACGTGCACAAAGTGTCCGGGACTGGACCTTGTGCTGGAGATT
TCGCCTTCCACAAGGAGGGCGCCTTTTTCCTGTACGACCGGCTCGCATCCACCGTGATCT
ATAGAGGAACCACCTTCGCGGAAGGAGTGGTCGCCTTCCTGATCCTGCCGCAAGCCAAGA
AGGACTTCTTTAGCTCCCATCCACTGCGCGAGCCCGTGAATGCCACCGAAGATCCCTCGT
CCGGCTATTACTCCACAACCATCCGGTACCAAGCCACCGGGTTCGGTACCAACGAGACTG
AGTACCTGTTCGAAGTCGACAACCTGACTTACGTGCAGCTCGAATCGCGGTTCACGCCAC
AGTTCCTCCTGCAACTCAACGAAACTATCTACACTAGCGGGAAACGCTCGAACACCACCGG
GAAGCTGATTTGGAAGGTCAACCCTGAAATCGACACCACCATCGGAGAGTGGGCTTTTTG
GGAGACTAAGAAGAACCTCACCCGGAAGATCAGATCCGAGGAATTGTCCTTCACCGTGGT
CAACACCCACCACCAGGATACTGGAGAGGAGTCCGCATCAAGCGGAAAGCTCGGCCTGAT
TACTAACACTATCGCGGGGGTCGCTGGATTGATTACCGGTGGTAGAAGGACCAGGAGAGA
GGCCATTGTGAACGCGCAGCCGAAGTGCAACCCTAATCTGCATTACTGGACCACTCAGGA
CGAGGGCGCCGCCATCGGCCTGGCCTGGATTCCGTACTTCGGCCCGGCCGCGGAAGGC
ATATACATTGAGGGACTTATGCACAATCAGGATGGACTGATTTGTGGTCTGCGCCAGCTGG
CCAACGAAACCACCCAGGCGCTGCAGCTGTTCCTCCGGGCAACCACCGAACTTCGCACTT
TCTCCATCCTGAACCGGAAGGCCATTGACTTCCTCTTGCAACGCTGGGGAGGAACTTGCC
ACATCCTGGGTCCTGATTGCTGCATCGAACCGCATGACTGGACAAAGAACATCACCGACAA
AATCGACCAGATCATCCACGATTTCGTGGACAAGACCCTGCCTGGATCAGGTTACATCCCC
GAGGCCCCGAGAGACGGCCAGGCCTACGTGCGGAAGGACGGCGAATGGGTCCTCCTGTC
CACCTTCCTTGGGACTATCGAAGGGCGCCACCATCACCACCACCATTGATAA
GP ΔTM-ΔMUC-T4 protein
(SEQ ID NO: 39)
MEFWLSWVFLVAILKGVQCVPLGVIHNSTLQVSDVDKLVCRDKLSSTNQLRSVGLN
LEGNGVATDVPSATKRWGFRSGVPPKVVNYEAGEWAENCYNLEIKKPDGSECLPAAPDGIRG
FPRCRYVHKVSGTGPCAGDFAFHKEGAFFLYDRLASTVIYRGTTFAEGVVAFLILPQAKKDFFS
SHPLREPVNATEDPSSGYYSTTIRYQATGFGTNETEYLFEVDNLTYVQLESRFTPQFLLQLNETI
YTSGKRSNTTGKLIWKVNPEIDTTIGEWAFWETKKNLTRKIRSEELSFTVVNTHHQDTGEESAS
SGKLGLITNTIAGVAGLITGGRRTRREAIVNAQPKCNPNLHYWTTQDEGAAIGLAWIPYFGPAAE
GIYIEGLMHNQDGLICGLRQLANETTQALQLFLRATTELRTFSILNRKAIDFLLQRWGGTCHILGP
DCCIEPHDWTKNITDKIDQIIHDFVDKTLPGSGYIPEAPRDGQAYVRKDGEWVLLSTFLGT
GP ΔTM-ΔMUC-T4 DNA
(SEQ ID NO: 40)
ATGGAGTTCTGGTTGTCTTGGGTGTTTCTCGTCGCTATTCTGAAGGGGGTGCAG
TGCGTGCCACTGGGAGTGATCCACAACAGCACTCTCCAAGTCTCCGACGTGGACAAGCTC
GTGTGCCGAGACAAGCTTTCCAGCACAAACCAGCTGCGGTCCGTGGGACTGAACCTGGAG
GGAAATGGCGTGGCCACCGATGTGCCGTCTGCAACCAAGAGATGGGGCTTCCGCTCGGG
CGTGCCCCCGAAAGTGGTCAACTACGAGGCCGGAGAATGGGCCGAAAACTGTTACAACCT
GGAGATCAAGAAGCCCGACGGTTCCGAATGCCTCCCTGCCGCCCCTGACGGAATCAGGG
GATTCCCGAGATGCCGCTACGTGCACAAAGTGTCCGGGACTGGACCTTGTGCTGGAGATT
TCGCCTTCCACAAGGAGGGCGCCTTTTTCCTGTACGACCGGCTCGCATCCACCGTGATCT
ATAGAGGAACCACCTTCGCGGAAGGAGTGGTCGCCTTCCTGATCCTGCCGCAAGCCAAGA
AGGACTTCTTTAGCTCCCATCCACTGCGCGAGCCCGTGAATGCCACCGAAGATCCCTCGT
CCGGCTATTACTCCACAACCATCCGGTACCAAGCCACCGGGTTCGGTACCAACGAGACTG
AGTACCTGTTCGAAGTCGACAACCTGACTTACGTGCAGCTCGAATCGCGGTTCACGCCAC
AGTTCCTCCTGCAACTCAACGAAACTATCTACACTAGCGGGAAACGCTCGAACACCACCGG
GAAGCTGATTTGGAAGGTCAACCCTGAAATCGACACCACCATCGGAGAGTGGGCTTTTTG
GGAGACTAAGAAGAACCTCACCCGGAAGATCAGATCCGAGGAATTGTCCTTCACCGTGGT
CAACACCCACCACCAGGATACTGGAGAGGAGTCCGCATCAAGCGGAAAGCTCGGCCTGAT
TACTAACACTATCGCGGGGGTCGCTGGATTGATTACCGGTGGTAGAAGGACCAGGAGAGA
GGCCATTGTGAACGCGCAGCCGAAGTGCAACCCTAATCTGCATTACTGGACCACTCAGGA
CGAGGGCGCCGCCATCGGCCTGGCCTGGATTCCGTACTTCGGCCCGGCCGCGGAAGGC
ATATACATTGAGGGACTTATGCACAATCAGGATGGACTGATTTGTGGTCTGCGCCAGCTGG
CCAACGAAACCACCCAGGCGCTGCAGCTGTTCCTCCGGGCAACCACCGAACTTCGCACTT
TCTCCATCCTGAACCGGAAGGCCATTGACTTCCTCTTGCAACGCTGGGGAGGAACTTGCC
ACATCCTGGGTCCTGATTGCTGCATCGAACCGCATGACTGGACAAAGAACATCACCGACAA
AATCGACCAGATCATCCACGATTTCGTGGACAAGACCCTGCCTGGATCAGGTTACATCCCC
GAGGCCCCGAGAGACGGCCAGGCCTACGTGCGGAAGGACGGCGAATGGGTCCTCCTGTC
CACCTTCCTTGGGACTTGATAA
GP ΔTM-ΔMUC-GCN4-X-HIS protein
(SEQ ID NO: 41)
MEFWLSWVFLVAILKGVQCVPLGVIHNSTLQVSDVDKLVCRDKLSSTNQLRSVGLN
LEGNGVATDVPSATKRWGFRSGVPPKVVNYEAGEWAENCYNLEIKKPDGSECLPAAPDGIRG
FPRCRYVHKVSGTGPCAGDFAFHKEGAFFLYDRLASTVIYRGTTFAEGVVAFLILPQAKKDFFS
SHPLREPVNATEDPSSGYYSTTIRYQATGFGTNETEYLFEVDNLTYVQLESRFTPQFLLQLNETI
YTSGKRSNTTGKLIWKVNPEIDTTIGEWAFWETKKNLTRKIRSEELSFTVVNTHHQDTGEESAS
SGKLGLITNTIAGVAGLITGGRRTRREAIVNAQPKCNPNLHYWTTQDEGAAIGLAWIPYFGPAAE
GIYIEGLMHNQDGLICGLRQLANETTQALQLFLRATTELRTFSILNRKAIDFLLQRWGGTCHILGP
DCCIEPHDWTKNITDKIDQIIHDFVDKTLPGSGKQIEDKIEEILSKIYHIENEIARIKKLIGIEGRHHH
HHH
GP ΔTM-ΔMUC-GCN4-X-HIS DNA
(SEQ ID NO: 42)
ATGGAGTTCTGGTTGTCTTGGGTGTTTCTCGTCGCTATTCTGAAGGGGGTGCAG
TGCGTGCCACTGGGAGTGATCCACAACAGCACTCTCCAAGTCTCCGACGTGGACAAGCTC
GTGTGCCGAGACAAGCTTTCCAGCACAAACCAGCTGCGGTCCGTGGGACTGAACCTGGAG
GGAAATGGCGTGGCCACCGATGTGCCGTCTGCAACCAAGAGATGGGGCTTCCGCTCGGG
CGTGCCCCCGAAAGTGGTCAACTACGAGGCCGGAGAATGGGCCGAAAACTGTTACAACCT
GGAGATCAAGAAGCCCGACGGTTCCGAATGCCTCCCTGCCGCCCCTGACGGAATCAGGG
GATTCCCGAGATGCCGCTACGTGCACAAAGTGTCCGGGACTGGACCTTGTGCTGGAGATT
TCGCCTTCCACAAGGAGGGCGCCTTTTTCCTGTACGACCGGCTCGCATCCACCGTGATCT
ATAGAGGAACCACCTTCGCGGAAGGAGTGGTCGCCTTCCTGATCCTGCCGCAAGCCAAGA
AGGACTTCTTTAGCTCCCATCCACTGCGCGAGCCCGTGAATGCCACCGAAGATCCCTCGT
CCGGCTATTACTCCACAACCATCCGGTACCAAGCCACCGGGTTCGGTACCAACGAGACTG
AGTACCTGTTCGAAGTCGACAACCTGACTTACGTGCAGCTCGAATCGCGGTTCACGCCAC
AGTTCCTCCTGCAACTCAACGAAACTATCTACACTAGCGGGAAACGCTCGAACACCACCGG
GAAGCTGATTTGGAAGGTCAACCCTGAAATCGACACCACCATCGGAGAGTGGGCTTTTTG
GGAGACTAAGAAGAACCTCACCCGGAAGATCAGATCCGAGGAATTGTCCTTCACCGTGGT
CAACACCCACCACCAGGATACTGGAGAGGAGTCCGCATCAAGCGGAAAGCTCGGCCTGAT
TACTAACACTATCGCGGGGGTCGCTGGATTGATTACCGGTGGTAGAAGGACCAGGAGAGA
GGCCATTGTGAACGCGCAGCCGAAGTGCAACCCTAATCTGCATTACTGGACCACTCAGGA
CGAGGGCGCCGCCATCGGCCTGGCCTGGATTCCGTACTTCGGCCCGGCCGCGGAAGGC
ATATACATTGAGGGACTTATGCACAATCAGGATGGACTGATTTGTGGTCTGCGCCAGCTGG
CCAACGAAACCACCCAGGCGCTGCAGCTGTTCCTCCGGGCAACCACCGAACTTCGCACTT
TCTCCATCCTGAACCGGAAGGCCATTGACTTCCTCTTGCAACGCTGGGGAGGAACTTGCC
ACATCCTGGGTCCTGATTGCTGCATCGAACCGCATGACTGGACAAAGAACATCACCGACAA
AATCGACCAGATCATCCACGATTTCGTGGACAAGACCCTGCCTGGGAGCGGCAAGCAAAT
CGAAGATAAAATCGAGGAAATTCTGTCAAAGATTTACCACATTGAGAACGAAATCGCCCGG
ATCAAGAAGCTGATAGGTATCGAGGGCCGGCATCACCACCACCATCATTGATAA
GP ΔTM-ΔMUC-GCN4 protein
(SEQ ID NO: 43)
MEFWLSWVFLVAILKGVQCVPLGVIHNSTLQVSDVDKLVCRDKLSSTNQLRSVGLN
LEGNGVATDVPSATKRWGFRSGVPPKVVNYEAGEWAENCYNLEIKKPDGSECLPAAPDGIRG
FPRCRYVHKVSGTGPCAGDFAFHKEGAFFLYDRLASTVIYRGTTFAEGVVAFLILPQAKKDFFS
SHPLREPVNATEDPSSGYYSTTIRYQATGFGTNETEYLFEVDNLTYVQLESRFTPQFLLQLNETI
YTSGKRSNTTGKLIWKVNPEIDTTIGEWAFWETKKNLTRKIRSEELSFTVVNTHHQDTGEESAS
SGKLGLITNTIAGVAGLITGGRRTRREAIVNAQPKCNPNLHYWTTQDEGAAIGLAWIPYFGPAAE
GIYIEGLMHNQDGLICGLRQLANETTQALQLFLRATTELRTFSILNRKAIDFLLQRWGGTCHILGP
DCCIEPHDWTKNITDKIDQIIHDFVDKTLPGSGKQIEDKIEEILSKIYHIENEIARIKKLIG
GP ΔTM-ΔMUC-GCN4 DNA
(SEQ ID NO: 44)
ATGGAGTTCTGGTTGTCTTGGGTGTTTCTCGTCGCTATTCTGAAGGGGGTGCAG
TGCGTGCCACTGGGAGTGATCCACAACAGCACTCTCCAAGTCTCCGACGTGGACAAGCTC
GTGTGCCGAGACAAGCTTTCCAGCACAAACCAGCTGCGGTCCGTGGGACTGAACCTGGAG
GGAAATGGCGTGGCCACCGATGTGCCGTCTGCAACCAAGAGATGGGGCTTCCGCTCGGG
CGTGCCCCCGAAAGTGGTCAACTACGAGGCCGGAGAATGGGCCGAAAACTGTTACAACCT
GGAGATCAAGAAGCCCGACGGTTCCGAATGCCTCCCTGCCGCCCCTGACGGAATCAGGG
GATTCCCGAGATGCCGCTACGTGCACAAAGTGTCCGGGACTGGACCTTGTGCTGGAGATT
TCGCCTTCCACAAGGAGGGCGCCTTTTTCCTGTACGACCGGCTCGCATCCACCGTGATCT
ATAGAGGAACCACCTTCGCGGAAGGAGTGGTCGCCTTCCTGATCCTGCCGCAAGCCAAGA
AGGACTTCTTTAGCTCCCATCCACTGCGCGAGCCCGTGAATGCCACCGAAGATCCCTCGT
CCGGCTATTACTCCACAACCATCCGGTACCAAGCCACCGGGTTCGGTACCAACGAGACTG
AGTACCTGTTCGAAGTCGACAACCTGACTTACGTGCAGCTCGAATCGCGGTTCACGCCAC
AGTTCCTCCTGCAACTCAACGAAACTATCTACACTAGCGGGAAACGCTCGAACACCACCGG
GAAGCTGATTTGGAAGGTCAACCCTGAAATCGACACCACCATCGGAGAGTGGGCTTTTTG
GGAGACTAAGAAGAACCTCACCCGGAAGATCAGATCCGAGGAATTGTCCTTCACCGTGGT
CAACACCCACCACCAGGATACTGGAGAGGAGTCCGCATCAAGCGGAAAGCTCGGCCTGAT
TACTAACACTATCGCGGGGGTCGCTGGATTGATTACCGGTGGTAGAAGGACCAGGAGAGA
GGCCATTGTGAACGCGCAGCCGAAGTGCAACCCTAATCTGCATTACTGGACCACTCAGGA
CGAGGGCGCCGCCATCGGCCTGGCCTGGATTCCGTACTTCGGCCCGGCCGCGGAAGGC
ATATACATTGAGGGACTTATGCACAATCAGGATGGACTGATTTGTGGTCTGCGCCAGCTGG
CCAACGAAACCACCCAGGCGCTGCAGCTGTTCCTCCGGGCAACCACCGAACTTCGCACTT
TCTCCATCCTGAACCGGAAGGCCATTGACTTCCTCTTGCAACGCTGGGGAGGAACTTGCC
ACATCCTGGGTCCTGATTGCTGCATCGAACCGCATGACTGGACAAAGAACATCACCGACAA
AATCGACCAGATCATCCACGATTTCGTGGACAAGACCCTGCCTGGGAGCGGCAAGCAAAT
CGAAGATAAAATCGAGGAAATTCTGTCAAAGATTTACCACATTGAGAACGAAATCGCCCGG
ATCAAGAAGCTGATAGGTTGATAA
GP ΔTM-T4-X-HIS protein
(SEQ ID NO: 45)
MEFWLSWVFLVAILKGVQCVPLGVIHNSTLQVSDVDKLVCRDKLSSTNQLRSVGLN
LEGNGVATDVPSATKRWGFRSGVPPKVVNYEAGEWAENCYNLEIKKPDGSECLPAAPDGIRG
FPRCRYVHKVSGTGPCAGDFAFHKEGAFFLYDRLASTVIYRGTTFAEGVVAFLILPQAKKDFFS
SHPLREPVNATEDPSSGYYSTTIRYQATGFGTNETEYLFEVDNLTYVQLESRFTPQFLLQLNETI
YTSGKRSNTTGKLIWKVNPEIDTTIGEWAFWETKKNLTRKIRSEELSFTVVSNGAKNISGQSPA
RTSSDPGTNTTTEDHKIMASENSSAMVQVHSQGREAAVSHLTTLATISTSPQSLTTKPGPDNST
HNTPVYKLDISEATQVEQHHRRTDNDSTASDTPSATTAAGPPKAENTNTSKSTDFLDPATTTSP
QNHSETAGNNNTHHQDTGEESASSGKLGLITNTIAGVAGLITGGRRTRREAIVNAQPKCNPNLH
YWTTQDEGAAIGLAWIPYFGPAAEGIYIEGLMHNQDGLICGLRQLANETTQALQLFLRATTELRT
FSILNRKAIDFLLQRWGGTCHILGPDCCIEPHDWTKNITDKIDQIIHDFVDKTLPGSGYIPEAPRD
GQAYVRKDGEWVLLSTFLGTIEGRHHHHHH
GP ΔTM-T4-X-HIS DNA
(SEQ ID NO: 46)
ATGGAGTTCTGGTTGTCTTGGGTGTTTCTCGTCGCTATTCTGAAGGGGGTGCAG
TGCGTGCCACTGGGAGTGATCCACAACAGCACTCTCCAAGTCTCCGACGTGGACAAGCTC
GTGTGCCGAGACAAGCTTTCCAGCACAAACCAGCTGCGGTCCGTGGGACTGAACCTGGAG
GGAAATGGCGTGGCCACCGATGTGCCGTCTGCAACCAAGAGATGGGGCTTCCGCTCGGG
CGTGCCCCCGAAAGTGGTCAACTACGAGGCCGGAGAATGGGCCGAAAACTGTTACAACCT
GGAGATCAAGAAGCCCGACGGTTCCGAATGCCTCCCTGCCGCCCCTGACGGAATCAGGG
GATTCCCGAGATGCCGCTACGTGCACAAAGTGTCCGGGACTGGACCTTGTGCTGGAGATT
TCGCCTTCCACAAGGAGGGCGCCTTTTTCCTGTACGACCGGCTCGCATCCACCGTGATCT
ATAGAGGAACCACCTTCGCGGAAGGAGTGGTCGCCTTCCTGATCCTGCCGCAAGCCAAGA
AGGACTTCTTTAGCTCCCATCCACTGCGCGAGCCCGTGAATGCCACCGAAGATCCCTCGT
CCGGCTATTACTCCACAACCATCCGGTACCAAGCCACCGGGTTCGGTACCAACGAGACTG
AGTACCTGTTCGAAGTCGACAACCTGACTTACGTGCAGCTCGAATCGCGGTTCACGCCAC
AGTTCCTCCTGCAACTCAACGAAACTATCTACACTAGCGGGAAACGCTCGAACACCACCGG
GAAGCTGATTTGGAAGGTCAACCCTGAAATCGACACCACCATCGGAGAGTGGGCTTTTTG
GGAGACTAAGAAGAACCTCACCCGGAAGATCAGATCCGAGGAATTGTCCTTCACTGTGGT
GTCCAACGGAGCGAAGAACATCAGCGGACAGTCCCCCGCACGGACCTCATCGGACCCGG
GAACCAACACCACCACCGAGGACCACAAGATCATGGCCTCGGAGAACTCATCCGCCATGG
TGCAAGTGCATAGCCAGGGCCGCGAAGCCGCCGTGTCCCACCTGACTACCCTGGCGACC
ATCAGCACCAGCCCGCAGTCGCTGACTACTAAGCCTGGGCCAGACAACAGCACCCACAAC
ACCCCTGTGTACAAGCTGGACATTTCCGAAGCTACTCAGGTCGAGCAGCATCACCGGCGG
ACCGACAACGATTCCACCGCTTCCGACACCCCCTCCGCCACCACCGCCGCCGGCCCGCC
GAAGGCCGAAAACACCAATACGTCAAAGTCGACCGATTTCCTTGACCCCGCCACCACTAC
GAGCCCCCAGAACCACTCAGAAACTGCGGGCAACAACAACACCCACCACCAGGATACTGG
AGAGGAGTCCGCATCAAGCGGAAAGCTCGGCCTGATTACTAACACTATCGCGGGGGTCGC
TGGATTGATTACCGGTGGTAGAAGGACCAGGAGAGAGGCCATTGTGAACGCGCAGCCGAA
GTGCAACCCTAATCTGCATTACTGGACCACTCAGGACGAGGGCGCCGCCATCGGCCTGGC
CTGGATTCCGTACTTCGGCCCGGCCGCGGAAGGCATATACATTGAGGGACTTATGCACAA
TCAGGATGGACTGATTTGTGGTCTGCGCCAGCTGGCCAACGAAACCACCCAGGCGCTGCA
GCTGTTCCTCCGGGCAACCACCGAACTTCGCACTTTCTCCATCCTGAACCGGAAGGCCATT
GACTTCCTCTTGCAACGCTGGGGAGGAACTTGCCACATCCTGGGTCCTGATTGCTGCATC
GAACCGCATGACTGGACAAAGAACATCACCGACAAAATCGACCAGATCATCCACGATTTCG
TGGACAAGACCCTGCCTGGATCAGGTTACATCCCCGAGGCCCCGAGAGACGGCCAGGCC
TACGTGCGGAAGGACGGCGAATGGGTCCTCCTGTCCACCTTCCTTGGGACTATCGAAGGG
CGCCACCATCACCACCACCATTGATAA
GP ΔTM-T4 protein
(SEQ ID NO: 47)
MEFWLSWVFLVAILKGVQCVPLGVIHNSTLQVSDVDKLVCRDKLSSTNQLRSVGLN
LEGNGVATDVPSATKRWGFRSGVPPKVVNYEAGEWAENCYNLEIKKPDGSECLPAAPDGIRG
FPRCRYVHKVSGTGPCAGDFAFHKEGAFFLYDRLASTVIYRGTTFAEGVVAFLILPQAKKDFFS
SHPLREPVNATEDPSSGYYSTTIRYQATGFGTNETEYLFEVDNLTYVQLESRFTPQFLLQLNETI
YTSGKRSNTTGKLIWKVNPEIDTTIGEWAFWETKKNLTRKIRSEELSFTVVSNGAKNISGQSPA
RTSSDPGTNTTTEDHKIMASENSSAMVQVHSQGREAAVSHLTTLATISTSPQSLTTKPGPDNST
HNTPVYKLDISEATQVEQHHRRTDNDSTASDTPSATTAAGPPKAENTNTSKSTDFLDPATTTSP
QNHSETAGNNNTHHQDTGEESASSGKLGLITNTIAGVAGLITGGRRTRREAIVNAQPKCNPNLH
YWTTQDEGAAIGLAWIPYFGPAAEGIYIEGLMHNQDGLICGLRQLANETTQALQLFLRATTELRT
FSILNRKAIDFLLQRWGGTCHILGPDCCIEPHDWTKNITDKIDQIIHDFVDKTLPGSGYIPEAPRD
GQAYVRKDGEWVLLSTFLGT
GP ΔTM-T4 DNA
(SEQ ID NO: 48)
ATGGAGTTCTGGTTGTCTTGGGTGTTTCTCGTCGCTATTCTGAAGGGGGTGCAG
TGCGTGCCACTGGGAGTGATCCACAACAGCACTCTCCAAGTCTCCGACGTGGACAAGCTC
GTGTGCCGAGACAAGCTTTCCAGCACAAACCAGCTGCGGTCCGTGGGACTGAACCTGGAG
GGAAATGGCGTGGCCACCGATGTGCCGTCTGCAACCAAGAGATGGGGCTTCCGCTCGGG
CGTGCCCCCGAAAGTGGTCAACTACGAGGCCGGAGAATGGGCCGAAAACTGTTACAACCT
GGAGATCAAGAAGCCCGACGGTTCCGAATGCCTCCCTGCCGCCCCTGACGGAATCAGGG
GATTCCCGAGATGCCGCTACGTGCACAAAGTGTCCGGGACTGGACCTTGTGCTGGAGATT
TCGCCTTCCACAAGGAGGGCGCCTTTTTCCTGTACGACCGGCTCGCATCCACCGTGATCT
ATAGAGGAACCACCTTCGCGGAAGGAGTGGTCGCCTTCCTGATCCTGCCGCAAGCCAAGA
AGGACTTCTTTAGCTCCCATCCACTGCGCGAGCCCGTGAATGCCACCGAAGATCCCTCGT
CCGGCTATTACTCCACAACCATCCGGTACCAAGCCACCGGGTTCGGTACCAACGAGACTG
AGTACCTGTTCGAAGTCGACAACCTGACTTACGTGCAGCTCGAATCGCGGTTCACGCCAC
AGTTCCTCCTGCAACTCAACGAAACTATCTACACTAGCGGGAAACGCTCGAACACCACCGG
GAAGCTGATTTGGAAGGTCAACCCTGAAATCGACACCACCATCGGAGAGTGGGCTTTTTG
GGAGACTAAGAAGAACCTCACCCGGAAGATCAGATCCGAGGAATTGTCCTTCACTGTGGT
GTCCAACGGAGCGAAGAACATCAGCGGACAGTCCCCCGCACGGACCTCATCGGACCCGG
GAACCAACACCACCACCGAGGACCACAAGATCATGGCCTCGGAGAACTCATCCGCCATGG
TGCAAGTGCATAGCCAGGGCCGCGAAGCCGCCGTGTCCCACCTGACTACCCTGGCGACC
ATCAGCACCAGCCCGCAGTCGCTGACTACTAAGCCTGGGCCAGACAACAGCACCCACAAC
ACCCCTGTGTACAAGCTGGACATTTCCGAAGCTACTCAGGTCGAGCAGCATCACCGGCGG
ACCGACAACGATTCCACCGCTTCCGACACCCCCTCCGCCACCACCGCCGCCGGCCCGCC
GAAGGCCGAAAACACCAATACGTCAAAGTCGACCGATTTCCTTGACCCCGCCACCACTAC
GAGCCCCCAGAACCACTCAGAAACTGCGGGCAACAACAACACCCACCACCAGGATACTGG
AGAGGAGTCCGCATCAAGCGGAAAGCTCGGCCTGATTACTAACACTATCGCGGGGGTCGC
TGGATTGATTACCGGTGGTAGAAGGACCAGGAGAGAGGCCATTGTGAACGCGCAGCCGAA
GTGCAACCCTAATCTGCATTACTGGACCACTCAGGACGAGGGCGCCGCCATCGGCCTGGC
CTGGATTCCGTACTTCGGCCCGGCCGCGGAAGGCATATACATTGAGGGACTTATGCACAA
TCAGGATGGACTGATTTGTGGTCTGCGCCAGCTGGCCAACGAAACCACCCAGGCGCTGCA
GCTGTTCCTCCGGGCAACCACCGAACTTCGCACTTTCTCCATCCTGAACCGGAAGGCCATT
GACTTCCTCTTGCAACGCTGGGGAGGAACTTGCCACATCCTGGGTCCTGATTGCTGCATC
GAACCGCATGACTGGACAAAGAACATCACCGACAAAATCGACCAGATCATCCACGATTTCG
TGGACAAGACCCTGCCTGGATCAGGTTACATCCCCGAGGCCCCGAGAGACGGCCAGGCC
TACGTGCGGAAGGACGGCGAATGGGTCCTCCTGTCCACCTTCCTTGGGACTTGATAA
GP ΔTM-GCN4-X-HIS protein
(SEQ ID NO: 49)
MEFWLSWVFLVAILKGVQCVPLGVIHNSTLQVSDVDKLVCRDKLSSTNQLRSVGLN
LEGNGVATDVPSATKRWGFRSGVPPKVVNYEAGEWAENCYNLEIKKPDGSECLPAAPDGIRG
FPRCRYVHKVSGTGPCAGDFAFHKEGAFFLYDRLASTVIYRGTTFAEGVVAFLILPQAKKDFFS
SHPLREPVNATEDPSSGYYSTTIRYQATGFGTNETEYLFEVDNLTYVQLESRFTPQFLLQLNETI
YTSGKRSNTTGKLIWKVNPEIDTTIGEWAFWETKKNLTRKIRSEELSFTVVSNGAKNISGQSPA
RTSSDPGTNTTTEDHKIMASENSSAMVQVHSQGREAAVSHLTTLATISTSPQSLTTKPGPDNST
HNTPVYKLDISEATQVEQHHRRTDNDSTASDTPSATTAAGPPKAENTNTSKSTDFLDPATTTSP
QNHSETAGNNNTHHQDTGEESASSGKLGLITNTIAGVAGLITGGRRTRREAIVNAQPKCNPNLH
YWTTQDEGAAIGLAWIPYFGPAAEGIYIEGLMHNQDGLICGLRQLANETTQALQLFLRATTELRT
FSILNRKAIDFLLQRWGGTCHILGPDCCIEPHDWTKNITDKIDQIIHDFVDKTLPGSGKQIEDKIEE
ILSKIYHIENEIARIKKLIGIEGRHHHHHH
GP ΔTM-GCN4-X-HIS DNA
(SEQ ID NO: 50)
ATGGAGTTCTGGTTGTCTTGGGTGTTTCTCGTCGCTATTCTGAAGGGGGTGCAG
TGCGTGCCACTGGGAGTGATCCACAACAGCACTCTCCAAGTCTCCGACGTGGACAAGCTC
GTGTGCCGAGACAAGCTTTCCAGCACAAACCAGCTGCGGTCCGTGGGACTGAACCTGGAG
GGAAATGGCGTGGCCACCGATGTGCCGTCTGCAACCAAGAGATGGGGCTTCCGCTCGGG
CGTGCCCCCGAAAGTGGTCAACTACGAGGCCGGAGAATGGGCCGAAAACTGTTACAACCT
GGAGATCAAGAAGCCCGACGGTTCCGAATGCCTCCCTGCCGCCCCTGACGGAATCAGGG
GATTCCCGAGATGCCGCTACGTGCACAAAGTGTCCGGGACTGGACCTTGTGCTGGAGATT
TCGCCTTCCACAAGGAGGGCGCCTTTTTCCTGTACGACCGGCTCGCATCCACCGTGATCT
ATAGAGGAACCACCTTCGCGGAAGGAGTGGTCGCCTTCCTGATCCTGCCGCAAGCCAAGA
AGGACTTCTTTAGCTCCCATCCACTGCGCGAGCCCGTGAATGCCACCGAAGATCCCTCGT
CCGGCTATTACTCCACAACCATCCGGTACCAAGCCACCGGGTTCGGTACCAACGAGACTG
AGTACCTGTTCGAAGTCGACAACCTGACTTACGTGCAGCTCGAATCGCGGTTCACGCCAC
AGTTCCTCCTGCAACTCAACGAAACTATCTACACTAGCGGGAAACGCTCGAACACCACCGG
GAAGCTGATTTGGAAGGTCAACCCTGAAATCGACACCACCATCGGAGAGTGGGCTTTTTG
GGAGACTAAGAAGAACCTCACCCGGAAGATCAGATCCGAGGAATTGTCCTTCACTGTGGT
GTCCAACGGAGCGAAGAACATCAGCGGACAGTCCCCCGCACGGACCTCATCGGACCCGG
GAACCAACACCACCACCGAGGACCACAAGATCATGGCCTCGGAGAACTCATCCGCCATGG
TGCAAGTGCATAGCCAGGGCCGCGAAGCCGCCGTGTCCCACCTGACTACCCTGGCGACC
ATCAGCACCAGCCCGCAGTCGCTGACTACTAAGCCTGGGCCAGACAACAGCACCCACAAC
ACCCCTGTGTACAAGCTGGACATTTCCGAAGCTACTCAGGTCGAGCAGCATCACCGGCGG
ACCGACAACGATTCCACCGCTTCCGACACCCCCTCCGCCACCACCGCCGCCGGCCCGCC
GAAGGCCGAAAACACCAATACGTCAAAGTCGACCGATTTCCTTGACCCCGCCACCACTAC
GAGCCCCCAGAACCACTCAGAAACTGCGGGCAACAACAACACCCACCACCAGGATACTGG
AGAGGAGTCCGCATCAAGCGGAAAGCTCGGCCTGATTACTAACACTATCGCGGGGGTCGC
TGGATTGATTACCGGTGGTAGAAGGACCAGGAGAGAGGCCATTGTGAACGCGCAGCCGAA
GTGCAACCCTAATCTGCATTACTGGACCACTCAGGACGAGGGCGCCGCCATCGGCCTGGC
CTGGATTCCGTACTTCGGCCCGGCCGCGGAAGGCATATACATTGAGGGACTTATGCACAA
TCAGGATGGACTGATTTGTGGTCTGCGCCAGCTGGCCAACGAAACCACCCAGGCGCTGCA
GCTGTTCCTCCGGGCAACCACCGAACTTCGCACTTTCTCCATCCTGAACCGGAAGGCCATT
GACTTCCTCTTGCAACGCTGGGGAGGAACTTGCCACATCCTGGGTCCTGATTGCTGCATC
GAACCGCATGACTGGACAAAGAACATCACCGACAAAATCGACCAGATCATCCACGATTTCG
TGGACAAGACCCTGCCTGGGAGCGGCAAGCAAATCGAAGATAAAATCGAGGAAATTCTGT
CAAAGATTTACCACATTGAGAACGAAATCGCCCGGATCAAGAAGCTGATAGGTATCGAGGG
CCGGCATCACCACCACCATCATTGATAA
GP ΔTM-GCN4 protein
(SEQ ID NO: 51)
MEFWLSWVFLVAILKGVQCVPLGVIHNSTLQVSDVDKLVCRDKLSSTNQLRSVGLN
LEGNGVATDVPSATKRWGFRSGVPPKVVNYEAGEWAENCYNLEIKKPDGSECLPAAPDGIRG
FPRCRYVHKVSGTGPCAGDFAFHKEGAFFLYDRLASTVIYRGTTFAEGVVAFLILPQAKKDFFS
SHPLREPVNATEDPSSGYYSTTIRYQATGFGTNETEYLFEVDNLTYVQLESRFTPQFLLQLNETI
YTSGKRSNTTGKLIWKVNPEIDTTIGEWAFWETKKNLTRKIRSEELSFTVVSNGAKNISGQSPA
RTSSDPGTNTTTEDHKIMASENSSAMVQVHSQGREAAVSHLTTLATISTSPQSLTTKPGPDNST
HNTPVYKLDISEATQVEQHHRRTDNDSTASDTPSATTAAGPPKAENTNTSKSTDFLDPATTTSP
QNHSETAGNNNTHHQDTGEESASSGKLGLITNTIAGVAGLITGGRRTRREAIVNAQPKCNPNLH
YWTTQDEGAAIGLAWIPYFGPAAEGIYIEGLMHNQDGLICGLRQLANETTQALQLFLRATTELRT
FSILNRKAIDFLLQRWGGTCHILGPDCCIEPHDWTKNITDKIDQIIHDFVDKTLPGSGKQIEDKIEE
ILSKIYHIENEIARIKKLIG
GP ΔTM-GCN4 DNA
(SEQ ID NO: 52)
ATGGAGTTCTGGTTGTCTTGGGTGTTTCTCGTCGCTATTCTGAAGGGGGTGCAG
TGCGTGCCACTGGGAGTGATCCACAACAGCACTCTCCAAGTCTCCGACGTGGACAAGCTC
GTGTGCCGAGACAAGCTTTCCAGCACAAACCAGCTGCGGTCCGTGGGACTGAACCTGGAG
GGAAATGGCGTGGCCACCGATGTGCCGTCTGCAACCAAGAGATGGGGCTTCCGCTCGGG
CGTGCCCCCGAAAGTGGTCAACTACGAGGCCGGAGAATGGGCCGAAAACTGTTACAACCT
GGAGATCAAGAAGCCCGACGGTTCCGAATGCCTCCCTGCCGCCCCTGACGGAATCAGGG
GATTCCCGAGATGCCGCTACGTGCACAAAGTGTCCGGGACTGGACCTTGTGCTGGAGATT
TCGCCTTCCACAAGGAGGGCGCCTTTTTCCTGTACGACCGGCTCGCATCCACCGTGATCT
ATAGAGGAACCACCTTCGCGGAAGGAGTGGTCGCCTTCCTGATCCTGCCGCAAGCCAAGA
AGGACTTCTTTAGCTCCCATCCACTGCGCGAGCCCGTGAATGCCACCGAAGATCCCTCGT
CCGGCTATTACTCCACAACCATCCGGTACCAAGCCACCGGGTTCGGTACCAACGAGACTG
AGTACCTGTTCGAAGTCGACAACCTGACTTACGTGCAGCTCGAATCGCGGTTCACGCCAC
AGTTCCTCCTGCAACTCAACGAAACTATCTACACTAGCGGGAAACGCTCGAACACCACCGG
GAAGCTGATTTGGAAGGTCAACCCTGAAATCGACACCACCATCGGAGAGTGGGCTTTTTG
GGAGACTAAGAAGAACCTCACCCGGAAGATCAGATCCGAGGAATTGTCCTTCACTGTGGT
GTCCAACGGAGCGAAGAACATCAGCGGACAGTCCCCCGCACGGACCTCATCGGACCCGG
GAACCAACACCACCACCGAGGACCACAAGATCATGGCCTCGGAGAACTCATCCGCCATGG
TGCAAGTGCATAGCCAGGGCCGCGAAGCCGCCGTGTCCCACCTGACTACCCTGGCGACC
ATCAGCACCAGCCCGCAGTCGCTGACTACTAAGCCTGGGCCAGACAACAGCACCCACAAC
ACCCCTGTGTACAAGCTGGACATTTCCGAAGCTACTCAGGTCGAGCAGCATCACCGGCGG
ACCGACAACGATTCCACCGCTTCCGACACCCCCTCCGCCACCACCGCCGCCGGCCCGCC
GAAGGCCGAAAACACCAATACGTCAAAGTCGACCGATTTCCTTGACCCCGCCACCACTAC
GAGCCCCCAGAACCACTCAGAAACTGCGGGCAACAACAACACCCACCACCAGGATACTGG
AGAGGAGTCCGCATCAAGCGGAAAGCTCGGCCTGATTACTAACACTATCGCGGGGGTCGC
TGGATTGATTACCGGTGGTAGAAGGACCAGGAGAGAGGCCATTGTGAACGCGCAGCCGAA
GTGCAACCCTAATCTGCATTACTGGACCACTCAGGACGAGGGCGCCGCCATCGGCCTGGC
CTGGATTCCGTACTTCGGCCCGGCCGCGGAAGGCATATACATTGAGGGACTTATGCACAA
TCAGGATGGACTGATTTGTGGTCTGCGCCAGCTGGCCAACGAAACCACCCAGGCGCTGCA
GCTGTTCCTCCGGGCAACCACCGAACTTCGCACTTTCTCCATCCTGAACCGGAAGGCCATT
GACTTCCTCTTGCAACGCTGGGGAGGAACTTGCCACATCCTGGGTCCTGATTGCTGCATC
GAACCGCATGACTGGACAAAGAACATCACCGACAAAATCGACCAGATCATCCACGATTTCG
TGGACAAGACCCTGCCTGGGAGCGGCAAGCAAATCGAAGATAAAATCGAGGAAATTCTGT
CAAAGATTTACCACATTGAGAACGAAATCGCCCGGATCAAGAAGCTGATAGGTTGATAA

The amino acid sequence of full-length Ebola virus glycoprotein, noted herein as “EBOV GP” or “Ebola virus GP” is additionally exemplified by the amino acid sequences found in GenBank as accession numbers AHX24649.1 and AHX24649.2. The term also encompasses Ebola virus GP or a fragment thereof coupled to, for example, a histidine tag (e.g. see accession number AHX24649.1 with a decahistidine tag, mouse or human Fc, or a signal sequence. The omission or inclusion (at any appropriate location within the amino acid sequence) and subsequent removal of purification tags such as histidine tags and the like is also contemplated herein.

“Trimeric polypeptide” or “trimeric conformation” as used herein refers to a variant of a GP sequence that has been modified to include a trimerization motif to enhance the formation of a native trimeric conformation of EBOV GP during expression and subsequent purification from a host cell. It also refers to a variant of a GP sequence in which the transmembrane domain has been deleted. As used herein, such a polypeptide “trimerizes” (i.e., polypeptide monomers form a trimeric conformation). Optionally, the GP sequence has been modified by deletion of the mucin-like domain and addition of a trimerization motif as described herein. Sequence motifs such as the trimerization motifs described and contemplated herein should show affinities between the outer faces of the polypeptide chain, whereby each chain has at least one surface area that has an affinity towards another surface area of the same chain (for example charge interaction, hydrophobic/hydrophilic). The organization of these affinity-providing sections of a polypeptide chain can be designed to facilitate formation of dimers, trimers and higher order protein complex structures. Thus, even entirely artificial and non-nature derived polymerization polypeptide sequences are contemplated. The GP constructs are made using standard molecular biological and recombinant DNA techniques known in the art.

As used herein, the mucin-like domain sequence, the T4 trimerization sequence, the GCN4 trimerization sequence and the transmembrane domain sequence are as follows.

Mucin-like domain sequence (SEQ ID NO: 53):
NGAKNISGQSPARTSSDPGTNTTTEDHKIMASENSSAMVQVHSQGREAAV
SHLTTLATISTSPQSLTTKPGPDNSTHNTPVYKLDISEATQVEQHHRRTD
NDSTASDTPSATTAAGPPKAENTNTSKSTDFLDPATTTSPQNHSETAGNN
T4 trimerization sequence (SEQ ID NO: 54):
GSGYIPEAPRDGQAYVRKDGEWVLLSTFLGT
GCN4 trimerization sequence (SEQ ID NO: 55):
GSGKQIEDKIEEILSKIYHIENEIARIKKLIG
Transmembrane domain sequence (SEQ ID NO: 56):
QWIPAGIGVTGVIIAVIALFCICKFVF

As described herein, GP fragments, analogs and variants are contemplated. Such fragments, analogs and variants include modifications relative to the wild-type GP sequence as well as modifications with respect to the transmembrane and mucin-like domains (e.g., deleting both more or less) and modifications related to the trimerization motif (e.g., adding different sequences). For example, immunogenic variants retain at least 90% amino acid identity over at least 10 contiguous amino acids of any GP antigen described herein, or at least 85% amino acid identity over at least 15 contiguous amino acids of the antigen. Other examples include at least 70%, 75%, 80%, 85%, 90%, 91%, 92%, 93%, 94%, 95%, 96%, 97%. 98%, or 99% identity over at least 50 contiguous amino acids of the antigen, or over at least 100, 200, 300, 400, 500 or 600 contiguous amino acids of the antigen. In one embodiment, an immunogenic variant has at least 70%, 75%, 80%, 85%, 90%, 91%, 92%, 93%, 94%, 95%, 96%, 97%. 98%, or 99% identity over the full length of a particular antigen. In some embodiments, the variant is a naturally occurring variant.

As another example, immunogenic fragments, and variants thereof, comprise at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 48, 50, 100, 200, 300, 400, 500 or 600 contiguous amino acids of the antigen. The immunogenic fragment may comprise any number of contiguous amino acids between the aforementioned amino acids. As described herein, in various embodiments, the immunogenic fragment is shorter than a full length (676aa) EBOV GP protein. In certain aspects, the immunogenic fragment is deleted for the transmembrane region and optionally the mucin-like domain as described herein. In exemplary embodiments disclosed herein, the fragments range in size from 472-633 amino acids in length as monomers. After intracellular processing and removal of the signal peptide, these protein constructs are secreted to the cell culture supernatant as monomers with lengths of 453-614 amino acids (i.e., 19 amino acids shorter).

As disclosed herein, suitable proteins include precursor proteins, mature proteins, fragments, fusion proteins and peptides. In the compositions, the proteins may be present in the same form or as a mixture of these forms. For cellular production of a glycoprotein, a signal peptide may be part of the precursor protein. It may also be desirable to use a protein without a transmembrane or intracellular region or both.

As discussed herein, one or more portions, also called fragments, of a glycoprotein are chosen for containing one or more epitopes that bind to neutralizing antibodies. Portions containing epitopes may be identified by an assay, such as inhibition of neutralizing antibodies on viral infection of cells.

Compositions that comprise at least one immunogenic fragment of an immunogenic EBOV GP polypeptide may be used as immunogens. In some embodiments, the immunogenic fragment is encoded by the recombinant expression vectors described herein. The immunogenic fragment may consist of at least 6, 10, 15, 20, 30, 40, 50, 60, 70, 80, 90, 100, 150, 200, 300, 400, 500 or 600 or more contiguous amino acids of an immunogenic polypeptide. The immunogenic fragment may comprise any number of contiguous amino acids between the aforementioned immunogenic polypeptide. The immunogenic fragments may comprise a sufficient number of contiguous amino acids that form a linear epitope and/or may comprise a sufficient number of contiguous amino acids that permit the fragment to fold in the same (or sufficiently similar) three-dimensional conformation as the full-length polypeptide from which the fragment is derived to present a non-linear epitope or epitopes (also referred to in the art as conformational epitopes). Assays for assessing whether the immunogenic fragment folds into a conformation comparable to the full-length polypeptide include, for example, the ability of the protein to react with mono- or polyclonal antibodies that are specific for native or unfolded epitopes, the retention of other ligand-binding functions, and the sensitivity or resistance of the polypeptide fragment to digestion with proteases (see, e.g., Sambrook et al., Molecular Cloning: A Laboratory Manual, 3d ed., Cold Spring Harbor Laboratory Press, NY (2001)). Accordingly, by way of example, the three-dimensional conformation of a polypeptide fragment is sufficiently similar to the full-length polypeptide when the capability to bind and the level of binding of an antibody that specifically binds to the full-length polypeptide is substantially the same for the fragment as for the full-length polypeptide (i.e., the level of binding has been retained to a statistically, clinically, and/or biologically sufficient degree compared with the immunogenicity of the exemplary or wild-type full-length antigen).

In various aspects of the present disclosure, the GP polypeptide or protein used comprises an amino acid sequence that is at least 80% or 90% (e.g., at least 90%, 91%, 92%, 93%, 94%, 95%, 96%, 97%, 98%, or 99% identical) to SEQ ID NOS: 1, 3, 5, 7, 9, 11, 13, 15, 17, 19, 21, 23, 25, 27, 29, 31, 33, 35, 37, 39, 41, 43, 45, 47, 49, or 51. Sequence variation can also be expressed as a limited number (e.g., 1, 2, 3, 4, 5, 6, 7, 8, 9, or 10) amino acid sequence differences between the wild type sequence and the aligned sequence used in the present disclosure.

### Polynucleotides, Vectors and Production of Polypeptides

The invention includes polynucleotides encoding the polypeptides described herein. Exemplary sequences are set out in SEQ ID NOs: 1-52. Because of the degeneracy of the genetic code, numerous polynucleotide sequences encode a given amino acid sequence, and all are contemplated as part of the invention. In some variations, codon selection is optimized for the type of host organism that will be used for expression.

In some embodiments, vectors are used to express the polynucleotides described herein. Expression vectors generally include expression control sequences selected for a type of host cell to be used for protein expression. In some embodiments, the expression vector is a mammalian expression vector or yeast expression vector.

In other aspects, the polypeptide described herein is produced by expression in a suitable prokaryotic or eukaryotic host system characterized by producing a pharmacologically acceptable protein molecule (e.g., capable of eliciting an immune response as described herein). Examples of eukaryotic cells are mammalian cells, such as CHO, COS, HEK 293, BHK, SK-Hep, NSO, SP2/0, and HepG2. Insect cells such as S2 cells, or SF-9 cells are also contemplated.

In certain embodiments of the present disclosure, cell culture utilizes genetically stable transformed recombinant host systems. In certain other embodiments, a transient expression system is used. Transient gene expression approaches are well known in the art and include, for example, the laboratory scale expression of proteins, such as antibodies, glycoproteins, enzymes, fusion proteins and molecular variants thereof. Technologies covering this have been described in detail for HEK293 cells, for CHO cells, and for insect cells. This includes technologies which can be scaled up under suspension cultures. [Jordan, M., et al. (1996) Transfecting mammalian cells: optimisation of critical parameters affecting calcium-phosphate precipitate formation. Nucleic Acids Res 24(4), pp. 596-601; Jordan, M., et al. (1998): Calcium-phosphate mediated DNA transfer into HEK-293 cells in suspension: control of physicochemical parameters allows transfection in stirred media. Transfection and protein expression in mammalian cells. Cytotechnology, 26(1), pp. 39-47; Batard, P., et al. (2001) Transfer of high copy number plasmid into mammalian cells by calcium phosphate transfection. Gene 270 (1-2), 61-68, Meissner, P., et al. (2001) Transient gene expression: recombinant protein production with suspension-adapted HEK293-EBNA cells. Biotechnol Bioeng 75(2), pp. 197-203; Girard, P. et al. (2001) Calcium phosphate transfection optimization for serum-free suspension culture Cytotechnology 35 (3), pp. 175-180; Girard, P et al. (2002) 100 Liter-transient transfection. Cytotechnology 38 (1), pp. 15-21; Lindell, J. et al. (2004) Calfection—a novel transfection method for mammalian cells cultivated in suspension, Biochim Biophys Acta Gene Structure and Expression 1676 (2), pp. 155-161; Derouazi, M. et al. (2004) Serum-free large scale transient transfection of CHO cells Biotechnol Bioeng 87 (4), pp. 538-545; Baldi, L. et al. (2005): Transient gene expression in suspension HEK-293 cells: application to large-scale protein production. Biotechnol. Prog.; 21(1); pp. 148-153; Hildinger, M., et al. (2007) High titer, serum-free production of adeno-associated virus vectors by polyethyleneimine-mediate plasmid transfection in mammalian suspension cells Biotechnol Lett, 29 (11), 1713-1721; Muller, N. et al. (2007): Scalable transient gene expression in Chinese hamster ovary cells in instrumented and non-instrumented cultivation systems, Biotechnol Lett, 29 (5), pp. 703-11; Stettler, M. et al. (2007). Novel orbital shake bioreactors for transient production of CHO derived IgGs. Biotechnol. Prog. 23 (6), pp. 1340-1346; Wulhfard, S., et al. (2008). Mild hypothermia improves yields several fold by transient gene expression in Chinese Hamster Ovary cells. Biotechnol. Prog. 2008; 24(2), pp. 458-465; Backliwal, G., et al. (2008). Valproic acid—a viable alternative to sodium butyrate for enhancing transient gene expression in mammalian cultures. Biotechnol. Bioeng. DOI 10.1002/bit.21882; Bertschinger, M. et al. (2008). The kinetics of polyethylenimine-mediated transfection in suspension cultures of Chinese hamster ovary cells. Mol. Biotechnol. 40 (2) 136-143; Backliwal, G; et al. (2008): Rational vector design and multi-pathway modulation of HEK 293 cells yield recombinant antibody titers exceeding 1 gram per liter by transient transfection under serum-free conditions Nucleic Acids Research 2008, 36, (15), e96; Backliwal, G, et al. (2008): Coexpression of acidic Fibroblast Growth Factor enhances specific productivity and antibody titers in transiently transfected HEK-293 cells. New Biotechnology 2008, 12, 162-166; Wulhfard, S. et al. (2010): Valproic acid enhances recombinant mRNA and protein levels in transiently transfected Chinese hamster ovary cells. J. Biotechnology (2010) doi: 10.1016/j.jbiotec. 2010.05.003; Shen, X., et al. (2014) Virus-free transient protein production in Sf9 cells. Journal of Biotechnology vol 171, 61-70 dx.doi.org/doi: 10.1016/j.jbiotec. 2013.11.018.]

As one embodiment, high-level expression of proteins from CHO cells has been developed at ExcellGene and has been improved from earlier technologies developed by leading pharmaceutical companies in the 1980s and 1990 (Wurm, F. M. (2004): Production of recombinant protein therapeutics in cultivated mammalian cells. Nature Biotechnology 22, 11, 1393-1398). Such technologies are used for the production of pharmaceutical protein products such as HERCEPTIN®, an antibody against breast cancer, HUMIRA® an antibody used in the treatment of rheumatoid arthritis, or PULMOZYME®, an enzyme for the treatment of cystic fibrosis and are typically executed at 1000-20′000 liter scale in stainless steel controlled bioreactors (M. De Jesus and F. M. Wurm (2011). Manufacturing Recombinant Proteins in kg-ton Quantities using Animal Cells in Bioreactors. Eur J Pharm Biopharm vol 78, 2, 184-188). These ExcellGene improved technologies include the use of ExcellGene's host cells that have been optimized for rapid growth (<16 h/doubling), selected under continuous culture without lag phases over many months. In addition to using the Transposase transfection approach described in the Examples (which favors integration of transgenic DNA into active chromatin), the cell culture approach for production is based on principles which are used for industrial scale up. The chosen cell host (CHOExpress™ cells, ExcellGene SA) and derived recombinant cell populations have characteristics of growing in single cell suspension to densities of 5-20×106 cells/ml in animal component free media under batch culture condition, have a high subcultivation rate capacity (1/20 or higher) and grow under fed-batch conditions over 14-20 days to 25-35×106 cells/ml while maintaining high viability. The batch and fed-batch principles can be used at small scale from the milliliter scale to the liter scale of operation without the use of instrumented (CO2, oxygen, pH-controlled, etc.) bioreactors, i.e. in orbitally shaken tubes and in cylindrical containers. With respect to the Examples disclosed herein, 100-500 ml productions were sufficient to produce the quantities of protein necessary, after transient or stable expression and purification, to characterize their responsiveness of these to the various sera from patients and to determine their structural features.

To the extent protein modifications related to purification are undesirable for a future vaccine (e.g., His tags), an alternative purification approach is contemplated. Likewise, where a clinical candidate is identified, the final antigen candidate can be produced in clonally derived cell lines that match expectations for clinical manufacture. Thus, recombinant CHO pools are generated to compare, for example the histidine tag-free (“His tag-free”) trimer containing DNA constructs, for expression and subsequent generation of high expressing clonally derived cell populations for cell culture upstream process development and for down-stream processing. An optimized manufacturing process is thus developed from such a clonally derived cell population, ready for Master Cell Bank generation and eventual production under cGMP.

A wide variety of vectors are used for the preparation of a GP protein and are selected from eukaryotic and prokaryotic expression vectors. Examples of vectors for prokaryotic expression include plasmids such as, and without limitation, pRSET, pET, and pBAD, wherein the promoters used in prokaryotic expression vectors include one or more of, and without limitation, lac, trc, trp, recA, or araBAD. Examples of vectors for eukaryotic expression include: (i) for expression in yeast, vectors such as, and without limitation, pAO, pPIC, pYES, or pMET, using promoters such as, and without limitation, AOX1, GAP, GAL1, or AUG1; (ii) for expression in insect cells, vectors such as and without limitation, pMT, pAc5, pIB, pMIB, or pBAC, using promoters such as and without limitation PH, p10, MT, Ac5, OpIE2, gp64, or polh, and (iii) for expression in mammalian cells, vectors such as and without limitation pSVL, pCMV, pRc/RSV, pcDNA3, or pBPV, and vectors derived from, in one aspect, viral systems such as and without limitation vaccinia virus, adeno-associated viruses, herpes viruses, or retroviruses, using promoters such as and without limitation CMV, SV40, EF-1, UbC, RSV, ADV, BPV, and □-actin.

Production of a protein described herein includes any method known in the art for (i) the production of recombinant DNA by genetic engineering, (ii) introducing recombinant DNA into prokaryotic or eukaryotic cells by, for example and without limitation, transfection, electroporation or microinjection, (iii) cultivating said transformed cells, (iv) expressing protein, e.g. constitutively or upon induction, and (v) isolating said protein, e.g. from the culture medium or by harvesting the transformed cells, in order to obtain purified protein.

The production process applied herein in various embodiments is based on principles applied under large-scale manufacturing approaches used in the pharmaceutical industry. The envisioned manufacturing process for the production of, for example, the GP1/2 variant protein with highest immunogenicity and having features of manufacturability, will be done in a fed-batch process that starts from a clonally derived cell line, of which frozen vials have been prepared under cGMP compliant conditions in a Master Cell Bank (MCB). Such a cell bank will consist of 300 or 500 vials. The MCB will be tested for freedom of adventitious agents, such as bacteria, viruses or mycoplasma.

The manufacturing process begins with the thawing of a vial and the revitalized cells are established as a seed train culture to eventually initiate from such cultures a production process in a larger bioreactor system. The seed train cultures are sub-cultivated every 3 to 4 days, with a seed density of 0.3-0.5×106 cells/mL and maintained until a manufacturing process in a larger bioreactor can be initiated. The cell culture media in such a process will be animal component-free or chemically defined. When a production process is envisioned, a culture from the seed train will be expanded through further sub-cultivation and scaling up the volume of culture until sufficient biomass has been generated to inoculate the final production vessel. The final production vessel can have a working volume between 10 liters and 1000 liters or even more, depending on the obtained volumetric yield from the clonally-derived cell line expressing the desired protein and the needs for the clinical trials and eventually for a market supply.

The inoculation density for the production process will, in one embodiment, be between 0.5 and 4×106 cells/mL and the production medium used will be animal component free or chemically defined. The inoculation into the production vessel may occur by simple transfer of the necessary volume of cell suspension and subsequent dilution of the culture with the fresh production medium. Otherwise it is also possible to transfer cells after a centrifugation step or a step that removes the inoculum medium prior to transfer of cells into the production vessel. In this case, the centrifuged cells are taken up into fresh medium and only then transferred.

The production culture will be monitored daily for growth and several metabolic parameters. The provisioning of certain feeds which include glucose will be done according to a prior developed optimal schedule which maximized product formation and product quality. The harvest of the supernatant containing the product of interest will be done when both product titer and product quality criteria are fulfilled.

Subsequently, the harvest fluid will be separated from cells and the clarified and sterile filtered product containing liquid will be exposed to several purification steps, typically at least 3 purification phases, which will include the use of chromatographic columns. These phases will eventually deliver a highly purified and stable protein product in a buffer (purified bulk) that can be used for further processing, for example for fill and finish, which will include the addition of a suitable vaccine adjuvant. The applied chromatography principles can include affinity chromatography, ionic exchange chromatography, size exclusion chromatography and others. The methods used have to assure a maximal removal of cell host derived contaminants and will also provide support for the assumption that unknown adventitious agents, theoretically present or accidentally introduced into the product containing fluid streams, will be removed or inactivated to a degree that satisfy today's known regulatory constraints, as they are defined by the FDA or the European EMA.

### Compositions and Formulations

The present disclosure includes compositions that comprise a EBOV GP described herein. In some embodiments, the composition is an antigenic composition. In some embodiments, the composition further comprises a pharmaceutically acceptable carrier. The term carrier encompasses diluents, excipients, adjuvants and combinations thereof. Pharmaceutically acceptable carriers are well known in the art (see, e.g., Remington's Pharmaceutical Sciences by Martin, 1975).

Exemplary “diluents” include sterile liquids such as sterile water, saline solutions, and buffers (e.g., phosphate, tris, borate, succinate, or histidine). Exemplary “excipients” are inert substances that may enhance vaccine stability and include but are not limited to polymers (e.g., polyethylene glycol), carbohydrates (e.g., starch, glucose, lactose, sucrose, or cellulose), and alcohols (e.g., glycerol, sorbitol, or xylitol).

The innate immune system comprises cells that provide defense in a non-specific manner to infection by other organisms. Innate immunity is an immediate defense but it is not long-lasting or protective against future challenges. Immune system cells that generally have a role in innate immunity are phagocytic, such as macrophages and dendritic cells. The innate immune system interacts with the adaptive (also called acquired) immune system in a variety of ways. Cells of the innate immune system can participate in antigen presentation to cells of the adaptive immune system, including expressing lymphokines that activate other cells, emitting chemotactic molecules that attract cells that may be specific to the invader, and secreting cytokines that recruit and activate cells of the adaptive immune system. The immunogenic/antigenic/vaccine compositions disclosed herein optionally include an agent that activates innate immunity in order to enhance the effectiveness of the composition.

Many types of agents can activate innate immunity. Organisms, like bacteria and viruses, can activate innate immunity, as can components of organisms, chemicals such as 2′-5′ oligo A, bacterial endotoxins, RNA duplexes, single stranded RNA and other molecules. Many of the agents act through a family of molecules—the Toll-like receptors (TLRs). Engaging a TLR can also lead to production of cytokines and chemokines and activation and maturation of dendritic cells, components involved in development of acquired immunity. The TLR family can respond to a variety of agents, including lipoprotein, peptidoglycan, flagellin, imidazoquinolines, CpG DNA, lipopolysaccharide and double stranded RNA (Akira et al. Biochemical Soc Transactions 31: 637-642, 2003). These types of agents are sometimes called pathogen (or microbe)-associated molecular patterns.

In one aspect, one or more adjuvants are included in the composition, in order to provide an agent(s) that activates innate immunity. An adjuvant is a substance incorporated into or administered simultaneously with antigen that increases the immune response. A variety of mechanisms have been proposed to explain how different adjuvants work (e.g., antigen depots, activators of dendritic cells, macrophages). Without wishing to be bound by theory, one mechanism involves activating the innate immune system, resulting in the production of chemokines and cytokines, which in turn activate the adaptive (acquired) immune response. In particular, some adjuvants activate dendritic cells through TLRs. Thus, an adjuvant is one type of agent that activates the innate immune system that may be used in a vaccine to EBOV. An adjuvant may act to enhance an acquired immune response in other ways too. Preferably the adjuvant is a TLR4 agonist.

One adjuvant that may be used in the compositions described herein is a monoacid lipid A (MALA) type molecule. An exemplary MALA is MPL® adjuvant as described in, e.g., Ulrich J. T. and Myers, K. R., “Monophosphoryl Lipid A as an Adjuvant” Chapter 21 in Vaccine Design, the Subunit and Adjuvant Approach, Powell, M. F. and Newman, M. J., eds. Plenum Press, N Y 1995. Another exemplary MALA is described by the chemical formula (I):

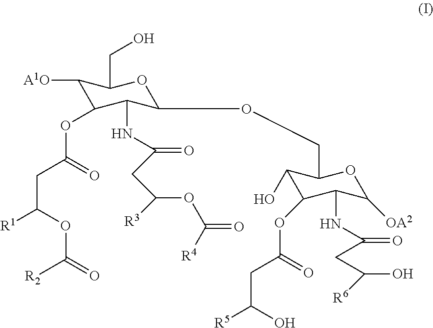

wherein the moieties A1 and A2 are independently selected from the group of hydrogen, phosphate, phosphate salts, carboxylate, carboxylate salts, sulfate, sulfate salts, sulfite, sulfite salts, aspartate, aspartate salts, succinate, succinate salts, carboxymethylphosphate and carboxymethylphosphate salts. Sodium and potassium are exemplary counterions for the phosphate and carboxylate salts. At least one of A1 and A2 is hydrogen. The moieties R1, R2, R3, R4, R5, and R6 are independently selected from the group of hydrocarbyl having 3 to 23 carbons, preferably a straight chain alkyl, represented by C3-C23. For added clarity it will be explained that when a moiety is “independently selected from” a specified group having multiple members, it should be understood that the member chosen for the first moiety does not in any way impact or limit the choice of the member selected for the second moiety. The carbon atoms to which R1, R3, R5 and R6 are joined are asymmetric, and thus may exist in either the R or S stereochemistry. In one embodiment all of those carbon atoms are in the R stereochemistry, while in another embodiment all of those carbon atoms are in the S stereochemistry.

“Hydrocarbyl” or “alkyl” refers to a chemical moiety formed entirely from hydrogen and carbon, where the arrangement of the carbon atoms may be straight chain or branched, noncyclic or cyclic, and the bonding between adjacent carbon atoms maybe entirely single bonds, i.e., to provide a saturated hydrocarbyl, or there may be double or triple bonds present between any two adjacent carbon atoms, i.e., to provide an unsaturated hydrocarbyl, and the number of carbon atoms in the hydrocarbyl group is between 3 and 24 carbon atoms. The hydrocarbyl may be an alkyl, where representative straight chain alkyls include methyl, ethyl, n-propyl, n-butyl, n-pentyl, n-hexyl, and the like, including undecyl, dodecyl, tridecyl, tetradecyl, pentadecyl, hexadecyl, heptadecyl, octadecyl, etc.; while branched alkyls include isopropyl, sec-butyl, isobutyl, tert-butyl, isopentyl, and the like. Representative saturated cyclic hydrocarbyls include cyclopropyl, cyclobutyl, cyclopentyl, cyclohexyl, and the like; while unsaturated cyclic hydrocarbyls include cyclopentenyl and cyclohexenyl, and the like. Unsaturated hydrocarbyls contain at least one double or triple bond between adjacent carbon atoms (referred to as an “alkenyl” or “alkynyl”, respectively, if the hydrocarbyl is non-cyclic, and cycloalkeny and cycloalkynyl, respectively, if the hydrocarbyl is at least partially cyclic). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like. For example, “C6-11 alkyl” mean an alkyl as defined above, containing from 6-11 carbon atoms, respectively.

The adjuvant of formula (I) may be obtained by synthetic methods known in the art, for example, the synthetic methodology disclosed in PCT International Publication No. WO 2009/035528, which is incorporated herein by reference, as well as the publications identified in WO 2009/035528, where each of those publications is also incorporated herein by reference. Certain of the adjuvants may also be obtained commercially. A preferred adjuvant is Product No. 699800 as identified in the catalog of Avanti Polar Lipids, Alabaster Ala., wherein R1, R3, R5 and R6 are undecyl and R2 and R4 are tridecyl.

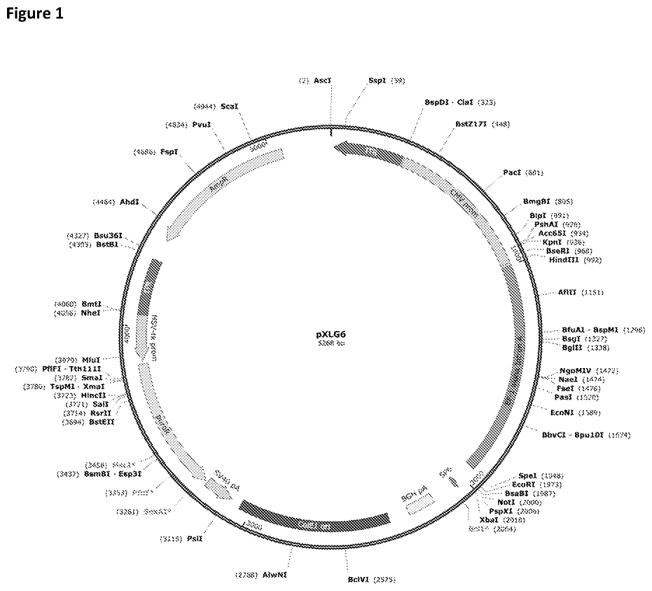

In various embodiments of the invention, the adjuvant has the chemical structure of formula (I) but the moieties A1, A2, R1, R2, R3, R4, R5, and R6 are selected from A1 being phosphate or phosphate salt and A2 is hydrogen; and R1, R3, R5 and R6 are selected from C7-C15 alkyl; and R2 and R4 are selected from C9-C17 hydrocarbyl. In a preferred embodiment of the invention, the GLA used in the examples herein has the structural formula set forth in FIG. 1, wherein R1, R3, R5 and R6 are undecyl and R2 and R4 are tridecyl.

The MALA adjuvants described above are a preferred adjuvant class for use in the immunogenic pharmaceutical compositions described herein. However, any of the following adjuvants may also be used alone, or in combination with an MALA adjuvant, in formulating an immunogenic pharmaceutical composition.

The adjuvant may be alum, where this term refers to aluminum salts, such as aluminum phosphate (AlPO4) and aluminum hydroxide (Al(OH)3). When alum is used as the adjuvant or as a co-adjuvant, the alum may be present in a dose of immunogenic pharmaceutical composition in an amount of about 100 to 1,000 μg, or 200 to 800 μg, or 300 to 700 μg or 400 to 600 μg. If the adjuvant of formula (1) is co-formulated with alum, the adjuvant of formula (1) is typically present in an amount less than the amount of alum, in various aspects the adjuvant of formula (1), on a weight basis, is present at 0.1-1%, or 1-5%, or 1-10%, or 1-100% relative to the weight of alum. In one aspect of the disclosure, the composition excludes the presence of alum.

The adjuvant may be an emulsion having vaccine adjuvant properties. Such emulsions include oil-in-water emulsions. Freund's incomplete adjuvant (IFA) is one such adjuvant. Another suitable oil-in-water emulsion is MF-59™ adjuvant which contains squalene, polyoxyethylene sorbitan monooleate (also known as Tween™ 80 surfactant) and sorbitan trioleate. Squalene is a natural organic compound originally obtained from shark liver oil, although also available from plant sources (primarily vegetable oils), including amaranth seed, rice bran, wheat germ, and olives. Other suitable emulsion adjuvants are Montanide™ adjuvants (Seppic Inc., Fairfield N.J.) including Montanide™ ISA 50V which is a mineral oil-based adjuvant, Montanide™ ISA 206, and Montanide™ IMS 1312. While mineral oil may be present in the adjuvant, in one embodiment, the oil component(s) of the compositions of the present invention are all metabolizable oils.

The adjuvant may be AS02™ adjuvant or AS04™ adjuvant. AS02™ adjuvant is an oil-in-water emulsion that contains both MPL™ adjuvant and QS-21™ adjuvant (a saponin adjuvant discussed elsewhere herein). AS04™ adjuvant contains MPL™ adjuvant and alum. The adjuvant may be Matrix-M™ adjuvant.

The adjuvant may be a saponin such as those derived from the bark of the Quillaja saponaria tree species, or a modified saponin, see, e.g., U.S. Pat. Nos. 5,057,540; 5,273,965; 5,352,449; 5,443,829; and 5,560,398. The product QS-21™ adjuvant sold by Antigenics, Inc. Lexington, Mass. is an exemplary saponin-containing co-adjuvant that may be used with the adjuvant of formula (1). Related to the saponins is the ISCOM™ family of adjuvants, originally developed by Iscotec (Sweden) and typically formed from saponins derived from Quillaja saponaria or synthetic analogs, cholesterol, and phospholipid, all formed into a honeycomb-like structure.

The adjuvant may be a cytokine that functions as an adjuvant, see, e.g., Lin R. et al. Clin. Infec. Dis. 21(6):1439-1449 (1995); Taylor, C. E., Infect. Immun. 63(9):3241-3244 (1995); and Egilmez, N. K., Chap. 14 in Vaccine Adjuvants and Delivery Systems, John Wiley & Sons, Inc. (2007). In various embodiments, the cytokine may be, e.g., granulocyte-macrophage colony-stimulating factor (GM-CSF); see, e.g., Change D. Z. et al. Hematology 9(3):207-215 (2004), Dranoff, G. Immunol. Rev. 188:147-154 (2002), and U.S. Pat. No. 5,679,356; or an interferon, such as a type I interferon, e.g., interferon-α (IFN-α) or interferon-β (IFN-β), or a type II interferon, e.g., interferon-γ (IFN-γ), see, e.g., Boehm, U. et al. Ann. Rev. Immunol. 15:749-795 (1997); and Theofilopoulos, A. N. et al. Ann. Rev. Immunol. 23:307-336 (2005); an interleukin, specifically including interleukin-1α (IL-1α), interleukin-1β (IL-1β), interleukin-2 (IL-2); see, e.g., Nelson, B. H., J. Immunol. 172(7):3983-3988 (2004); interleukin-4 (IL-4), interleukin-7 (IL-7), interleukin-12 (IL-12); see, e.g., Portielje, J. E., et al., Cancer Immunol. Immunother. 52(3): 133-144 (2003) and Trinchieri. G. Nat. Rev. Immunol. 3(2):133-146 (2003); interleukin-15 (Il-15), interleukin-18 (IL-18); fetal liver tyrosine kinase 3 ligand (Flt3L), or tumor necrosis factor α (TNFα).

The adjuvant may be unmethylated CpG dinucleotides, optionally conjugated to the antigens described herein.

Examples of immunopotentiators that may be used in the practice of the methods described herein as co-adjuvants include: MPL™; MDP and derivatives; oligonucleotides; double-stranded RNA; alternative pathogen-associated molecular patterns (PAMPS); saponins; small-molecule immune potentiators (SMIPs); cytokines; and chemokines.

In various embodiments, the co-adjuvant is MPL™ adjuvant, which is commercially available from GlaxoSmithKline (originally developed by Ribi ImmunoChem Research, Inc. Hamilton, Mont.). See, e.g., Ulrich and Myers, Chapter 21 from Vaccine Design: The Subunit and Adjuvant Approach, Powell and Newman, eds. Plenum Press, New York (1995). Related to MPL™ adjuvant, and also suitable as co-adjuvants for use in the compositions and methods described herein, are AS02™ adjuvant and AS04™ adjuvant. AS02™ adjuvant is an oil-in-water emulsion that contains both MPL™ adjuvant and QS-21™ adjuvant (a saponin adjuvant discussed elsewhere herein). AS04™ adjuvant contains MPL™ adjuvant and alum. MPL™ adjuvant is prepared from lipopolysaccharide (LPS) of Salmonella minnesota R595 by treating LPS with mild acid and base hydrolysis followed by purification of the modified LPS.

When two adjuvants are utilized in combination, the relative amounts of the two adjuvants may be selected to achieve the desired performance properties for the composition which contains the adjuvants, relative to the antigen alone. For example, the adjuvant combination may be selected to enhance the antibody response of the antigen, and/or to enhance the subject's innate immune system response. Activating the innate immune system results in the production of chemokines and cytokines, which in turn may activate an adaptive (acquired) immune response. An important consequence of activating the adaptive immune response is the formation of memory immune cells so that when the host re-encounters the antigen, the immune response occurs quicker and generally with better quality.

The adjuvant(s) may be pre-formulated prior to their combination with the EBOV proteins described herein. In one embodiment, an adjuvant may be provided as a stable aqueous suspension of less than 0.2 um and may further comprise at least one component selected from the group consisting of phospholipids, fatty acids, surfactants, detergents, saponins, fluorodated lipids, and the like. The adjuvant(s) may be formulated in an oil-in-water emulsion in which the adjuvant is incorporated in the oil phase. For use in humans, the oil is preferably metabolizable. The oil may be any vegetable oil, fish oil, animal oil or synthetic oil; the oil should not be toxic to the recipient and is capable of being transformed by metabolism. Nuts (such as peanut oil), seeds, and grains are common sources of vegetable oils. Particularly suitable metabolizable oils include squalene (2,6,10,15,19,23-hexamethyl-2,6,10,14,18,22-tetracosahexane), an unsaturated oil found in many different oils, and in high quantities in shark-liver oil. Squalene is an intermediate in the biosynthesis of cholesterol. In addition, the oil-in-water emulsions typically comprise an antioxidant, such as alpha-tocopherol (vitamin E, U.S. Pat. Nos. 5,650,155, 6,623,739). Stabilizers, such as a triglyceride, ingredients that confer isotonicity, and other ingredients may be added. An exemplary oil-in-water emulsion using squalene is known as “SE” and comprises squalene, glycerol, phosphatidylcholine or lecithin or other block co-polymer as a surfactant in an ammonium phosphate buffer, pH 5.1, with alpha-toceraphol.

The method of producing oil-in-water emulsions is well known to a person skilled in the art. Commonly, the method comprises mixing the oil phase with a surfactant, such as phosphatidylcholine, poloxamer, block co-polymer, or a TWEEN80® solution, followed by homogenization using a homogenizer. For instance, a method that comprises passing the mixture one, two, or more times through a syringe needle is suitable for homogenizing small volumes of liquid. Equally, the emulsification process in a microfluidizer (M110S microfluidics machine, maximum of 50 passes, for a period of 2 min at maximum pressure input of 6 bar (output pressure of about 850 bar)) can be adapted to produce smaller or larger volumes of emulsion. This adaptation can be achieved by routine experimentation comprising the measurement of the resultant emulsion until a preparation was achieved with oil droplets of the desired diameter. Other equipment or parameters to generate an emulsion may also be used. Disclosures of emulsion compositions, and method of their preparation, may be found in, e.g., U.S. Pat. Nos. 5,650,155; 5,667,784; 5,718,904; 5,961,970; 5,976,538; 6,572,861; and 6,630,161.

In still other embodiments of the present disclosure, virus-like particles may be used adjuvants with the antigenic or vaccine compositions described herein. Virus-like particles (VLPs) consist of one or more viral coat proteins that assemble into particles. They can be taken up by antigen presenting cells (APC), peptides derived from them are presented on MHC class I molecules at the cell surface, and thereby prime a CD8+ T cell response, either against the particle-forming protein itself or additional peptide sequences that are produced as fusions with the particle-forming protein.

Methods of Inducing an Immune Response to Prevent Disease

The present disclosure includes methods for eliciting an immune response in a subject, comprising administering to the subject an effective amount of an antigenic composition or vaccine composition comprising one or more of the EBOV GP described herein. Unless otherwise indicated, the antigenic composition is an immunogenic composition. The methods include administration of an antigenic or vaccine composition to a subject wherein the subject has not previously been infected with EBOV. Additionally, the methods include administration of an antigenic or vaccine composition to a subject wherein the subject is infected by EBOV and optionally experiencing one or more symptoms of EBOV infection.

The immune response raised by the methods of the present disclosure generally includes an antibody response, preferably a neutralizing antibody response, antibody dependent cell-mediated cytotoxicity (ADCC), antibody cell-mediated phagocytosis (ADCP), complement dependent cytotoxicity (CDC), and T cell-mediated response such as CD4+, CD8+. The immune response generated by the polypeptides and compositions disclosed herein generates an immune response that recognizes, and preferably ameliorates and/or neutralizes, Ebola virus. Methods for assessing antibody responses after administration of an antigenic composition (immunization or vaccination) are known in the art and/or described herein. In some embodiments, the immune response comprises a T cell-mediated response (e.g., peptide-specific response such as a proliferative response or a cytokine response). In preferred embodiments, the immune response comprises both a B cell and a T cell response. Antigenic compositions can be administered in a number of suitable ways, such as intramuscular injection, subcutaneous injection, intradermal administration and mucosal administration such as oral or intranasal. Additional modes of administration include but are not limited to intranasal administration, intra-vaginal, intra-rectal, and oral administration. A combination of different routes of administration in the immunized subject, for example intramuscular and intranasal administration at the same time, is also contemplated by the disclosure.

Antigenic compositions may be used to vaccinate both children and adults, including pregnant women. Thus, a subject may be less than 1 year old, 1-5 years old, 5-15 years old, 15-55 years old, or at least 55 years old. Preferred subjects for receiving the vaccines are the elderly (e.g., >55 years old, >60 years old, preferably >65 years old), and the young (e.g., <6 years old, 1-5 years old, preferably less than 1 year old). Additional subjects for receiving the vaccines or compositions of the disclosure include naïve (versus previously infected) subjects, currently infected subjects, or immunocompromised subjects.

Administration can involve a single dose or a multiple dose schedule. Multiple doses may be used in a primary immunization schedule and/or in a booster immunization schedule. In a multiple dose schedule the various doses may be given by the same or different routes, e.g., a parenteral prime and mucosal boost, or a mucosal prime and parenteral boost. Administration of more than one dose (typically two doses) is particularly useful in immunologically naive subjects or subjects of a hyporesponsive population (e.g., diabetics, or subjects with chronic kidney disease (e.g., dialysis patients)). Multiple doses will typically be administered at least 1 week apart (e.g., about 2 weeks, about 3 weeks, about 4 weeks, about 6 weeks, about 8 weeks, about 10 weeks, about 12 weeks, or about 16 weeks). Preferably multiple doses are administered from one, two, three, four or five months apart. Antigenic compositions of the present disclosure may be administered to patients at substantially the same time as (e.g., during the same medical consultation or visit to a healthcare professional) other vaccines.

In general, the amount of protein in each dose of the antigenic composition is selected as an amount effective to induce an immune response in the subject, without causing significant, adverse side effects in the subject. Preferably the immune response elicited includes: neutralizing antibody response; antibody dependent cell-mediated cytotoxicity (ADCC); antibody cell-mediated phagocytosis (ADCP); complement dependent cytotoxicity (CDC); T cell-mediated response such as CD4+, CD8+, or a protective antibody response. Protective in this context does not necessarily require that the subject is completely protected against infection. A protective response is achieved when the subject is protected from developing symptoms of disease, especially severe disease associated with the pathogen corresponding to the heterologous antigen. As described above, the immune response generated by the composition comprising EBOV GP as disclosed herein generates an immune response that recognizes, and preferably ameliorates and/or neutralizes, Ebola virus.

The amount of antigen (e.g., EBOV GP) can vary depending upon which antigenic composition is employed. Generally, it is expected that each human dose will comprise 0.1-2000 μg of protein (e.g., EBOV GP), such as from about 1 μg to about 2000 μg, for example, from about 1 μg to about 1500 μg, or from about 1 μg to about 1000 μg, or from about 1 μg to about 500 μg, or from about 1 μg to about 100 μg. In some embodiments, the amount of the protein is within any range having a lower limit of 0.1, 1, 5, 10, 20, 30, 40, 50, 60, 70, 80, 90, 100, 110, 120, 130, 140, 150, 160, 170, 180, 190, 200, 210, 220, 230, 240 or 250 μg, and an independently selected upper limit of 2000, 1950, 1900, 1850, 1800, 1750, 1700, 1650, 1600, 1550, 1500, 1450, 1400, 1350, 1300 or 1250, 1200, 1150, 1100, 1050, 1000, 950, 900, 850, 800, 750, 700, 650, 600, 550, 500, 450, 400, 350, 300 or 250 μg, provided that the lower limit is less than the upper limit. Generally, a human dose will be in a volume of from 0.1 ml to 1 ml, preferably from 0.25 ml to 0.5 ml. The amount utilized in an antigenic composition is selected based on the subject population. An optimal amount for a particular composition can be ascertained by standard studies involving observation of antibody titers and other responses (e.g., antigen-induced cytokine secretion) in subjects. Following an initial vaccination, subjects can receive a boost in about 4-12 weeks.

### EXAMPLES

Trimeric surface glycoproteins are a common target of neutralizing antibodies at the surface of numerous viruses. Recombinant trimers that closely mimic native surface glycoproteins are currently being evaluated as candidate immunogens for HIV, RSV and others, showing impressive results in terms of induction of neutralizing antibodies (nAbs) [Sullivan, Jonathan T., et al. “High-throughput protein engineering improves the antigenicity and stability of soluble HIV-1 envelope glycoprotein SOSIP trimers.” Journal of virology (2017): JVI-00862; Kulp, Daniel W., et al. “Structure-based design of native-like HIV-1 envelope trimers to silence non-neutralizing epitopes and eliminate CD4 binding.” Nature communications 8.1 (2017): 1655; McLellan, Jason S., et al. “Structure-based design of a fusion glycoprotein vaccine for respiratory syncytial virus.” Science 342.6158 (2013): 592-598.]. However, it is known that the recombinant EBOV GP protein is extremely unstable and adopts a monomeric state in solution, which is structurally distinct from the native trimer. The present disclosure and following examples thus describe stabilized, near-native EBOV GP trimers showing superior antigenic profiles than those of the monomeric wild type protein. Through biochemical and immunological profiling of these candidates, the importance of suitable antigen conformation for induction of neutralizing antibodies has been supported. The recombinant Ebola GP trimers presented herein thus have the potential to be recognized as valuable antigen candidates for the development of an efficacious EBOV vaccine that is currently being tested in pre-clinical animal models for immunogenicity and induction of superior neutralizing antibody responses.

### Example 1

### Ebola GP Production and Purification

Construction of expression vectors, CHO host cells, vectors and transfection of CHO cells, production of EBOLA GP1/2 protein variants.

### Cells

Chinese Hamster Ovary cells “CHOExpress™” (ExcellGene SA, Monthey, Switzerland), adapted to suspension culture and growing in animal component free media were subcultivated in ProCHO5 medium (Lonza) every 3 or 4 days under orbital shaking at 180 rpm in 50 ml OrbShake tubes (TubeSpin bioreactor 50, TPP, Trasadingen) in an incubator shaker (Kuhner Shaker) set to 37° C. and 5% CO₂. Cells grow to densities of 4-8×10⁶ cells/ml under these conditions.

### Vectors and Transfections

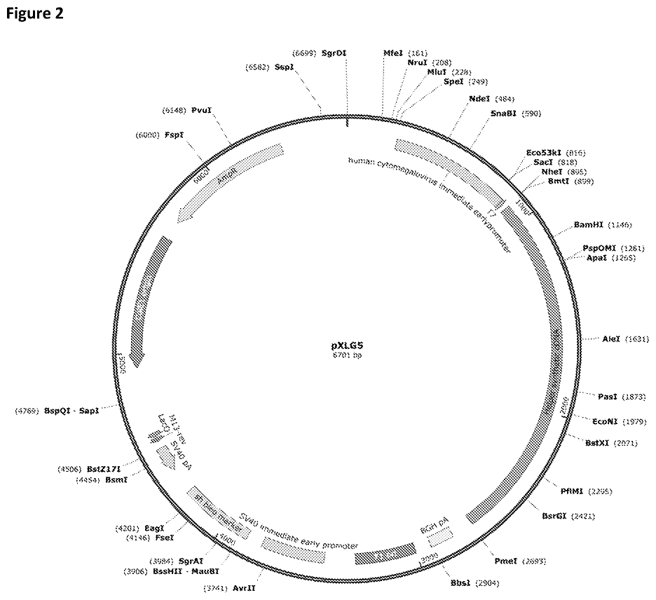

Transfections with cells from expanded seed train cultures of CHOExpress™ cells were executed using ExcellGene's transposon-based gene expression vector system, which consists of two plasmids, a gene-of-interest vector pXLG6 and a “mobilizing vector” pXLG5. These two vectors are co-transfected to obtain stable recombinant CHO cell lines. The gene-of-interest vector, pXLG6, places the DNA of interest, together with a resistance marker gene (puromycin resistance), in an expression cassette framed by piggyBac terminal repeats (Fraser et al. 1983, J. Virology 47 (2) 287-300). DNA in transfections constituted pXLG6 based vectors (ExcellGene SA, see FIG. 1) into which several EBOLA GP1/2 variant encoding DNA constructs were inserted. In the pXLG 6 map—ITR in the plasmid map indicate the positions of the piggyBac terminal repeat sequences. 2% of the transfected DNA contained the pXLG5 vector (FIG. 2), which encodes the piggyBac transposase. The transfections were executed using ExcellGene's CHO4Tx® transfection kit (ExcellGene SA, Monthey, Switzerland) and following the manufacturers' recommendation. Short term “transient” expression of the piggyBac transposase in animal cells will result in excision of the terminal repeat framed DNA from the pXLG6 vector and subsequent integration of the excised DNA into the genome of the cells. It has been shown that piggyBac transposase mobilized DNA is preferentially integrated into AT-rich regions of the host genome.

Puromycin resistant cell populations were selected and expanded for analysis and production of recombinant proteins expressed (recombinant CHO cell pools).

### EBOLA GP1/2 Construct Variants for Expression

26 different GP1/2 constructs were used in transient and stable transfections for evaluations after productions in CHO cells of the respective EBOLA surface protein variants.

A simplified and acronym-based representation of the various constructs used is given below (Table 1). Briefly, all constructs had the DNA for the transmembrane and intracellular tail sequence of the protein deleted (“ATM”), facilitating the secretion of the products into the culture supernatant of the cell culture.

Also deleted was in most constructs the “mucin” region of the protein (“ΔMUC”), a highly glycosylated region of the surface protein of the virus, thought to prevent efficient immune recognition in infected patients. The furin-cleavage site was left untouched in most cases, since it was assumed the intracellular cleavage at this position is an essential step in the appropriate folding of the GP1/2 protein complex. The “CC” labelled constructs contain additional cysteine encoding DNA in specific sites of the protein, thought to enhance the stability of the protein when paired by covalent cysteine di-sulfide bridges.

The “T4” and “GCN4” labelled constructs contain short stretches of additional DNA sequences which were expected to facilitate the assembly of monomeric GP units into trimeric structures. If trimerized as soluble molecule complexes, such a structure would more closely mimic the structure of GP1/2 as they are presented on the surface of the EBOLA virus. The T4 and GCN4 sequences are naturally derived protein sequences (Meier et al. 2004, J. Mol. Biol. 344, 1051-1069, Oshaben et al. 2012, Biochemistry 51 (47), 9581-9591). The sequences, both for protein and DNA, for all the constructs applied in this work are provided herein. The DNA sequences were cloned into the SpeI-EcoRI site of the pXLG6 vector.

Some constructs were designed to contain an artificial protease recognition sequence, susceptible to cleavage by a Factor Xa protease (“X”).

Also, DNA constructs used for transfection contained “His-tag” encoding DNA (“HIS”), expected to be useful in the purification of protein product from the supernatants of cell cultures.

TABLE 1
A simple word- and acronym-based representation for the desired protein variants, encoded by corresponding DNA vectors,
used in transient expression work and in stable transfections for the establishment of stable recombinant CHO cell pools.

| # | CONSTRUCT EXPLANATION | ACRONYM | SQ ID# |
|---|---|---|---|
| 1 | GP-DEL TRANSMEMBRANE, XA, HIS-TAG | GP ΔTM-X-HIS | 1 |
| 2 | GP-DEL TRANSMEMBRANE, DEL FURIN, XA, HIS-TAG | GP ΔTM-ΔFUR-X-HIS | 9 |
| 3 | GP-DEL TRANSMEMBRANE, DEL MUCIN, XA, HIS-TAG | GP ΔTM-ΔMUC-X-HIS | 3 |
| 4 | GP-DEL TRANSMEMBRANE, DEL MUCIN, DEL FURIN, XA, HIS-TAG | GP ΔTM-ΔMUC-ΔFUR-X-HIS | 11 |
| 5 | GP-DEL TRANSMEMBRANE | GP ΔTM | 13 |
| 6 | GP-DEL TRANSMEMBRANE, DEL MUCIN | GP ΔTM-ΔMUC | 15 |
| 7 | GP-DEL TRANSMEMBRANE, DEL MUCIN, XA, TRIMERT4, HIS-TAG | GP ΔTM-ΔMUC-X-T4-HIS | 5 |
| 8 | GP-DEL TRANSMEMBRANE, DEL MUCIN, XA, TRIMERGCN4, HIS-TAG | GP ΔTM-ΔMUC-X-GCN4-HIS | 7 |
| 9 | GP-DEL TRANSMEMBRANE, DEL MUCIN, CYS-CYS1, XA, HIS-TAG | GP ΔTM-ΔMUC-CC1-X-HIS | 17 |
| 10 | GP-DEL TRANSMEMBRANE, DEL MUCIN, CYS-CYS2, XA, HIS-TAG | GP ΔTM-ΔMUC-CC2-X-HIS | 19 |
| 11 | GP-DEL TRANSMEMBRANE, DEL MUCIN, CYS-CYS3, XA, HIS-TAG | GP ΔTM-ΔMUC-CC3-X-HIS | 21 |
| 12 | GP-DEL TRANSMEMBRANE, DEL MUCIN, CYS-CYS4, XA, HIS-TAG | GP ΔTM-ΔMUC-CC4-X-HIS | 23 |
| 13 | GP-DEL TRANSMEMBRANE, DEL MUCIN, CYS-CYS5, XA, HIS-TAG | GP ΔTM-ΔMUC-CC5-X-HIS | 25 |
| 14 | GP-DEL TRANSMEMBRANE, DEL MUCIN, CYS-CYS6, XA, HIS-TAG | GP ΔTM-ΔMUC-CC6-X-HIS | 27 |
| 15 | GP-DEL TRANSMEMBRANE, DEL MUCIN, CYS-CYS7, XA, HIS-TAG | GP ΔTM-ΔMUC-CC7-X-HIS | 29 |
| 16 | GP-DEL TRANSMEMBRANE, DEL MUCIN, CYS-CYS8, XA, HIS-TAG | GP ΔTM-ΔMUC-CC8-X-HIS | 31 |
| 17 | GP-DEL TRANSMEMBRANE, DEL MUCIN, CYS-CYS9, XA, HIS-TAG | GP ΔTM-ΔMUC-CC9-X-HIS | 33 |
| 18 | GP-DEL TRANSMEMBRANE, DEL MUCIN, CYS-CYS10, XA, HIS-TAG | GP ΔTM-ΔMUC-CC10-X-HIS | 35 |
| 19 | GP-DEL TRANSMEMBRANE, DEL MUCIN, TRIMERT4, XA, HIS-TAG | GP ΔTM-ΔMUC-T4-X-HIS | 37 |
| 20 | GP-DEL TRANSMEMBRANE, DEL MUCIN, TRIMERT4 | GP ΔTM-ΔMUC-T4 | 39 |
| 21 | GP-DEL TRANSMEMBRANE, DEL MUCIN, TRIMERGCN4, XA, HIS-TAG | GP ΔTM-ΔMUC-GCN4-X-HIS | 41 |
| 22 | GP-DEL TRANSMEMBRANE, DEL MUCIN, TRIMERGCN4 | GP ΔTM-ΔMUC-GCN4 | 43 |
| 23 | GP-DEL TRANSMEMBRANE, TRIMERT4, XA, HIS-TAG | GP ΔTM-T4-X-HIS | 45 |
| 24 | GP-DEL TRANSMEMBRANE, TRIMERT4 | GP ΔTM-T4 | 47 |
| 25 | GP-DEL TRANSMEMBRANE, TRIMERGCN4, XA, HIS-TAG | GP ΔTM-GCN4-X-HIS | 49 |
| 26 | GP-DEL TRANSMEMBRANE, TRIMERGCN4 | GP ΔTM-GCN4 | 51 |

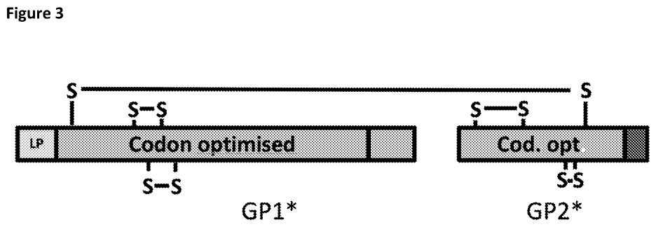

The constructs number 20 and 22 were found to be most promising candidate molecules for a future subunit vaccine. The construct GP ΔTM-ΔMUC-T4 (FIG. 3) is the most preferred protein for establishing a stable clonally obtained cell line and subsequent large-scale processes for eventual cGMP manufacturing of protein.

### Results

As described herein, the GP variants were all produced in Chinese hamster ovary (CHO) cells [De Jesus et al “Manufacturing recombinant proteins in kg-ton quantities using animal cells in bioreactors.” Eur J of Pharm and Biopharm 78(0.2), (2011].): 184-188]. Since one of the major complexities of EBOV GP is the presence of a heavy glycosylated mucin-like domain that appears to shield the virus from efficient humoral responses in infected people, mutant constructs with deletion of the mucin-like domain (GPΔTM-ΔMUC) were also considered for production. These constructs mimic the proteolysis step that occurs in vivo, thus enhancing the presentation of critical neutralization epitopes while removing epitopes located in the glycan cap and mucin-like domain that are mostly linear and non-protective [Davidson, Edgar, et al, J virol, 89(21) “Mechanism of binding to Ebola virus glycoprotein by the ZMapp, ZMAb, and MB-003 cocktail antibodies.” J Virology (2015].): JVI-01490. Davidson et al, J Virol, 89(21), 2015]. Some recombinant proteins have a C-terminal His-tag (“HIS”), which was introduced to facilitate the downstream purification strategy.

On the Ebola virus surface, three GP proteins non-covalently interact in a highly glycosylated chalice-like shaped trimer. For this reason, additional variants of GP variants were engineered by fusing trimerization motifs to their C-terminal ends to stabilize and/or facilitate the formation of GP-trimers. The majority of efforts were focused on mucin-deleted versions of GP because of its higher reactivity with all the antibodies tested, as discussed herein. Specifically, a trimerization domain from the C-terminus of bacteriophage T4 fibritin (T4) [Yang, Xinzhen, et al. “Highly stable trimers formed by human immunodeficiency virus type 1 envelope glycoproteins fused with the trimeric motif of T4 bacteriophage fibritin.” Journal of virology 76.9 (2002): 4634-4642. Yang et al, J Virol 76(9), 2002]; Zhao, Yuguang, et al. “Toremifene interacts with and destabilizes the Ebola virus glycoprotein.” Nature 535.7610 (2016): 169] and on the sequence of the GCN4 transcription factor (GCN4) [Yang, Xinzhen, et al. “Characterization of stable, soluble trimers containing complete ectodomains of human immunodeficiency virus type 1 envelope glycoproteins.” Journal of virology 74.12 (2000): 5716-5725. Yang et al, J Virol 74(12), 2000] [Zhao et al, Nature, 535(7610), 2016] was used.

### Example 2

### Characterization of EBOV GP Variants

When presented on the Ebola virus surface, the GP protein adopts a trimeric conformation. In order to favor the induction of the trimeric configuration, many variants of GP ΔTM were critically designed and produced by ExcellGene, as described herein. A structure-based design approach was applied to generate stabilized native-like EBOV-GP that will induce superior antibody neutralizing responses in vivo and become valuable candidates for and efficacious EBOV vaccine. This effort was mainly focused on the mucin-deleted version of GP because of its higher signal recognition with both the human and mouse antibodies tested (as shown herein).

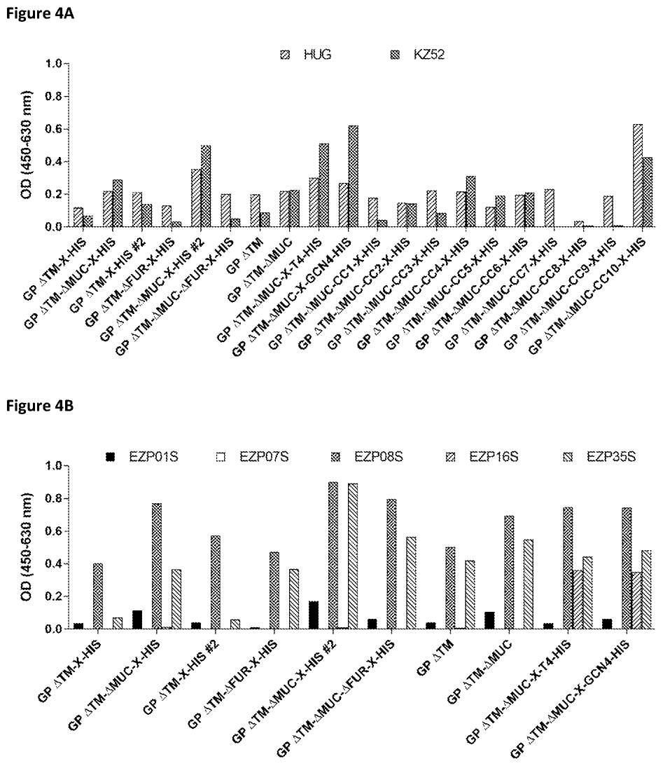

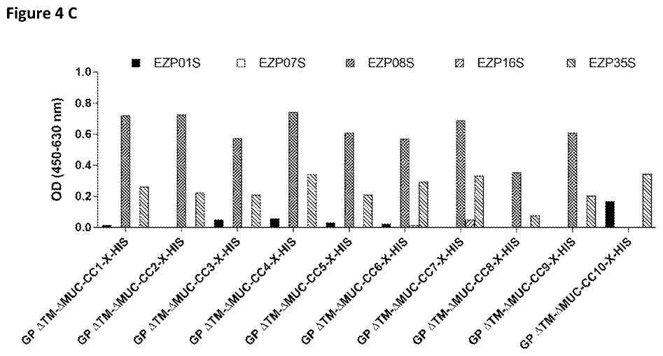

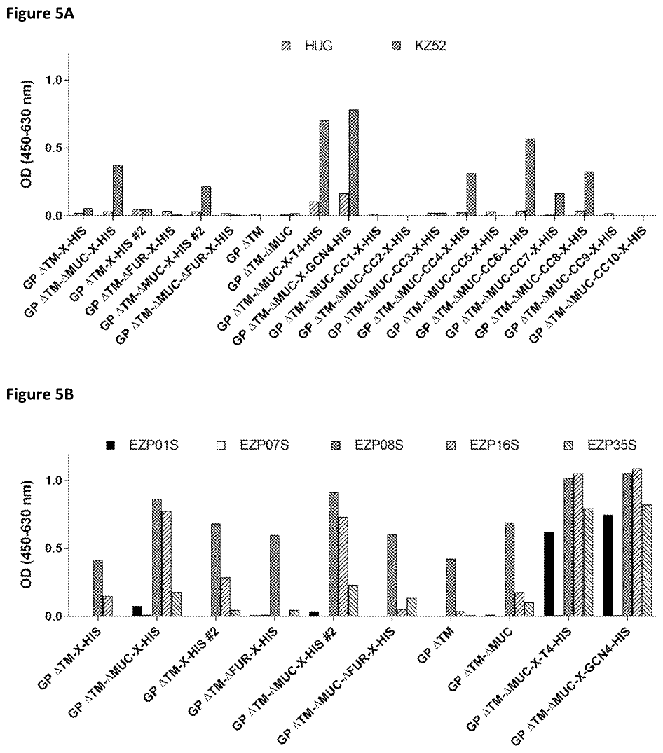

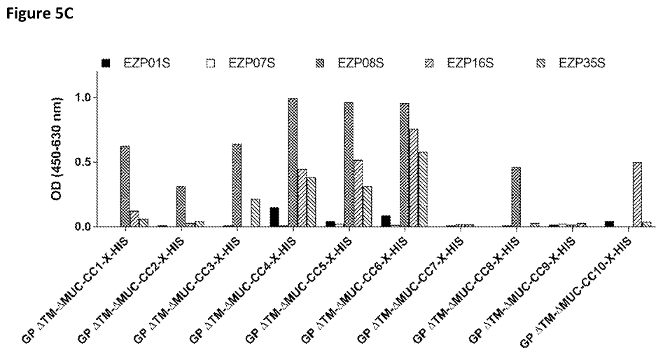

The performances of constructs 1 to 18 (Table 1), non-purified from the respective culture supernatants, were compared with the original GP ΔTM-X-HIS and GP ΔTM-ΔMUC-X-HIS products, both purified in PBS 1× (first two entries in each graph in FIGS. 4 and 5).

Constructs were analyzed in direct ELISA with the panel of human and mouse antibodies available. As shown in FIG. 4, KZ52 and HUG reacted with GP ΔTM-X-HIS #2 and GP ΔTM-ΔMUC-X-HIS #2 in a similar way respective to the purified GP ΔTM-X-HIS and GP ΔTM-ΔMUC-X-HIS, conferring significance to the production process. The signal from the new constructs were higher, and this may be due to the fact that new constructs were non-purified, thus their quantification could have been over-estimated because of other proteins/molecules in culture supernatant.

Similarly, a strong signal was registered for the two constructs containing the C-terminal trimerization domain. In addition, the construct with the disulphide bond GP ΔTM-ΔMUC-CC10-X-HIS showed to be highly recognized by KZ52 and, especially, by HUG.

The corresponding direct ELISA evaluation performed with the mouse mAbs provided by CEA showed that the GP ΔTM-ΔMUC-X-T4-HIS and GP ΔTM-ΔMUC-X-GCN4-HIS constructs were the only ones recognized also by the neutralizing mAb 16S, which might be an indication of a most near-native conformation of these proteins.

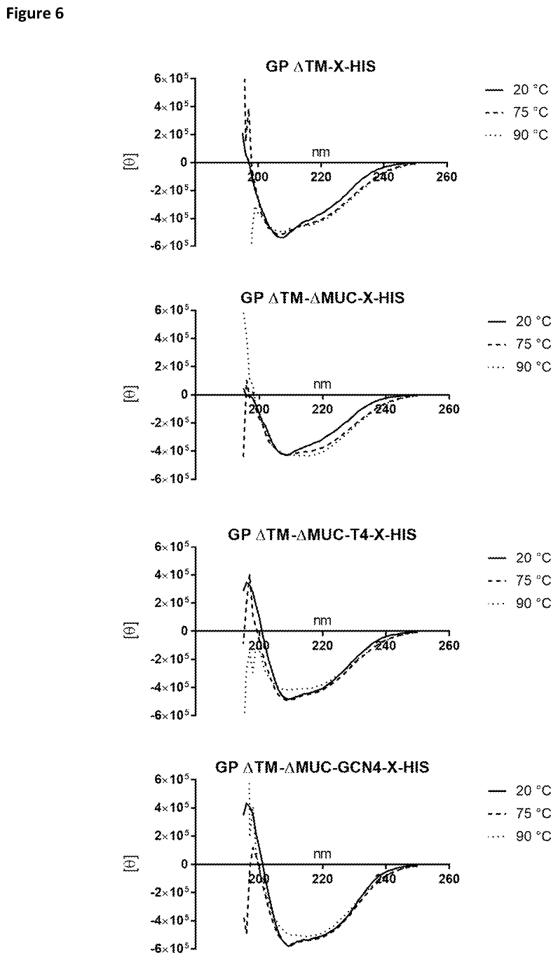

Similarly to examples presented herein, to test if adsorption to the amino-binding plate was affecting protein structure thus interfering with mAb recognition, the GP constructs were tested against the same panel of human and mouse (FIG. 6) antibodies in a sandwich ELISA configuration.

In this work, using His-tagged molecules, GP ΔTM-ΔMUC-X-T4-HIS and GP ΔTM-ΔMUC-X-GCN4-HIS were identified as the most interesting constructs to be tested in comparison with the original monomers GP ΔTM-X-HIS and GP ΔTM-ΔMUC-X-HIS. Other constructs that showed interesting profiles (GP ΔTM-ΔMUC-CC4-X-HIS to GP ΔTM-ΔMUC-CC6-X-HIS) were not selected because of their strong similarity with the monomer GP ΔTM-ΔMUC-X-HIS.

### Example 3

### Biochemical Characterization of GP ΔTM-X-HIS, GP ΔTM-ΔMUC-X-HIS, GP ΔTM-ΔMUC-T4-X-HIS, GP ΔTM-ΔMUC-GCN4-X-HIS

### Molecular Mass Determination

Multi-angle light scattering was used to assess the monodispersity and molecular weight of the proteins. Between 50-100 μg of the proteins were separated on a Superose™ 6 increase 10/300 GL column (GE Healthcare) using a HPLC system (Ultimate 3000, Thermo Scientific) coupled in-line to a multi-angel light scattering device (miniDAWN TREOS, Wyatt). Static light-scattering signal was recorded from three different scattering angles. The scatter data were analyzed by ASTRA software (version 6.1, Wyatt). Dn/dc values for the various proteins were determined theoretically according to the molecular mass and the amount of O- and N-glycosylation sites in each protein sequence.

### Circular Dichroism Studies

Circular dichroism (CD) spectra of peptides were recorded on a JASCO J-815 spectrometer (JASCO Corporation, Tokyo, Japan) equipped with a temperature controller and a 0.1 cm path length cuvette. The measurements were made in water at pH 7.3 and 22° C. and at proteins concentration of 250 μg/ml. For thermal stability profiles, spectra of peptides were registered from 20 to 90° C., at 10° C. intervals. The data were normalized to protein concentrations and expressed in units of molar residue ellipticity. Data analysis and display were done using GraphPad Prism 7 software.

### Fab Production

For Fab expression, heavy and light chain DNA sequences of KZ52 [Maruyama, Toshiaki, et al. “Recombinant human monoclonal antibodies to Ebola virus.” The Journal of infectious diseases179.Supplement_1 (1999): S235-S239.], mAb114, and mAb100 [Corti, Davide, et al. “Protective monotherapy against lethal Ebola virus infection by a potently neutralizing antibody.” Science (2016): aad5224; Misasi, John, et al. “Structural and molecular basis for Ebola virus neutralization by protective human antibodies.” Science 351.6279 (2016): 1343-1346] were cloned separately into pHLsec mammalian expression vectors [Aricescu, A. Radu, Weixian Lu, and E. Yvonne Jones. “A time- and cost-efficient system for high-level protein production in mammalian cells.” Acta Crystallographica Section D: Biological Crystallography 62.10 (2006): 1243-1250.]. Expression plasmids were co-transfected into HEK293-F cells in FreeStyle™ medium (Gibco™) using Polyethylenimine (Polysciences) transfection [Muller, N, et al. “Orbital shaker technology for the cultivation of mammalian cells in suspension.” Biotechnology and bioengineering 89.4 (2005): 400-406; Backliwal, G, et al. “High-density transfection with HEK-293 cells allows doubling of transient titers and removes need for a priori DNA complex formation with PEI.” Biotechnology and bioengineering 99.3 (2008): 721-727; Backliwal, G, et al. “Valproic acid: a viable alternative to sodium butyrate for enhancing protein expression in mammalian cell cultures.” Biotechnology and bioengineering 101.1 (2008): 182-189.]. Supernatants were harvested after 1 week by centrifugation and purified using a HiTrap™ Kappa select or Lambda select HP column (GE Healthcare). Elution of bound proteins was conducted using a 0.1 M glycine buffer (pH 2.7) and eluates were immediately neutralized by the addition of 1 M TRIS ethylamine (pH 9). The eluted Fabs were further purified by size exclusion chromatography on a Superdex 200 10/300 GL column (GE Healthcare).

### SPR Sensor Chip Preparation and Interaction Assays

SPR measurements were performed with Biacore 8K instrument (GE), at 25° C., with a flow rate of 30 μl min⁻¹. CM5 sensor chips series S, running buffer (HBS-EP+) and Amine Coupling Kit (1-ethyl-3-(3-dimethylaminopropyl) carbodiimide hydrochloride (EDC), N-hydroxysuccinimide (NHS), 1.0 M ethanolamine-HCl pH 8.5) were purchased from GE. The binding properties of the produced fAbs to the different Ebola GP proteins were evaluated upon immobilization of the proteins onto CM5 chip surface in flow cell 2 (FC-2), using Biacore Amine Coupling Kit according to the manufacturer's instructions. Each protein was preliminary diluted to 5 μg/ml in acetate buffer (pH 5) and 300 to 1,000 RU (Response Units) of protein (depending on the specific protein) were immobilized onto the chip surface. Blank amine immobilization was performed on flow cell 1 (FC-1), used as reference. Evaluation of antigen-antibody affinity was performed with Fabs diluted in HBS-EP+ buffer 1× at the desired concentrations and injected over the functionalized surface with an injection time of 120 s and dissociation time of 600 s. Surface regeneration was performed with MgCl2 3 M (GE). Surface regeneration conditions were evaluated and established after appropriate pH scouting experiments.

All sensograms were corrected by subtracting the signal recorded on FC-2 (functionalized cell) from the signal recorded of FC-1 (reference cell); collected data were evaluated by non-linear analysis of the association and dissociation curves using SPR kinetic evaluation software (BIAevaluation Software, version 2.0, Biacore). Data fitting was carried out with a 1:1 Langmuir binding model to obtain kₐ, k_{d} and K_{D}=k_{d}/kₐ. Fitting analysis was evaluated for all the performed assays.

### Results

Considering the importance of having a fully characterized protein for data interpretation and future (pre)clinical studies, the native/non-native structure of the constructs and their monomeric/trimeric form were studied. A panel of orthogonal techniques (size exclusion chromatography, circular dichroism, and light scattering) have been applied to further characterize the structural integrity of the recombinant proteins and to check their ability to assume a correct conformation. In addition, SPR measurements were performed to evaluate antigen-antibody affinities.

The determination of the absolute molecular weight of four EBOV GP variants (GP ΔTM-X-HIS, GP ΔTM-ΔMUC-X-HIS, GP ΔTM-ΔMUC-T4-X-HIS, GP ΔTM-ΔMUC-GCN4-X-HIS)—the most promising in terms of antigenicity—provided the analytical evidence that inclusion of the T4 or GCN4 trimerization domains at the C-terminus of the GP sequence drove the trimerization of the GP ΔTM-ΔMUC-X-HIS protein, as highlighted in Table 2.

TABLE 2
SEC-MALS absolute determination
of the proteins' molecular weight

| | PEAK 1 | PEAK 2 |
|---|---|---|
| GP ΔTM-X-HIS | 196 KDA | — |
| GP ΔTM- ΔMUC-X-HIS | 240 KDA | 97 KDA |
| GP ΔTM- ΔMUC-T4-X-HIS | 316 KDA | — |
| GP ΔTM- ΔMUC-GCN4-X-HIS | 296 KDA | — |

The secondary structure profiles of the four GP proteins GP ΔTM-X-HIS, GP ΔTM-ΔMUC-X-HIS, GP ΔTM-ΔMUC-T4-X-HIS, and GP ΔTM-ΔMUC-GCN4-X-HIS were acquired. As shown in FIG. 6, the CD profiles evidenced the presence of a major component of α-helix in the secondary structure of the proteins, as expected from literature data for class I fusion proteins such as Ebola GP [Colman, Peter M., and Michael C. Lawrence. “The structural biology of type I viral membrane fusion.” Nature Reviews Molecular Cell Biology 4.4 (2003): 309.]. Interestingly, the two bands of the CD α-helical component were more evident in the case of the two trimeric proteins, suggesting that trimerization favored the retention of native secondary structure.

Changes in the secondary structure of the proteins as a function of the temperature were also investigated and, as depicted in FIG. 6, GP ΔTM-ΔMUC-T4-X-HIS, and GP ΔTM-ΔMUC-GCN4-X-HIS appeared to be stable up to 75° C. and showed the lowest impact in structural changes at the various temperatures tested, probably because of the considerable amount of disulfide bonds known to stabilize the GP structure at each monomer's level [Lee, J. E., et al., Structure of the Ebola virus glycoprotein bound to an antibody from a human survivor. Nature, 2008. 454(7201): p. 177].

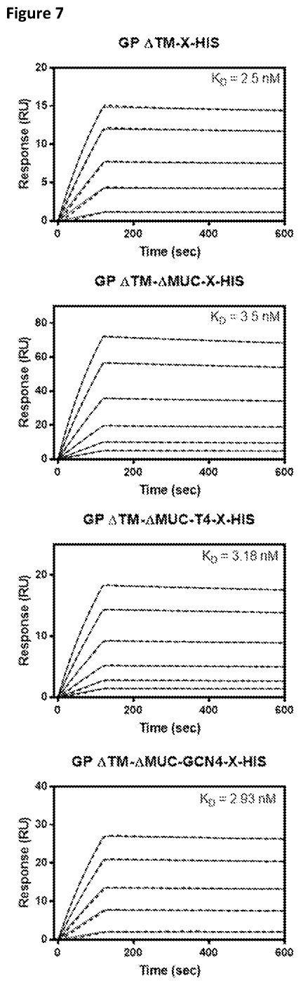

SPR experiments were performed for evaluation of epitopes availability and affinities among GP antigens and the monoclonal and neutralizing antibody mAb114 [Corti, Davide, et al. “Protective monotherapy against lethal Ebola virus infection by a potently neutralizing antibody.” Science (2016): aad5224; Misasi, John, et al. “Structural and molecular basis for Ebola virus neutralization by protective human antibodies.” Science 351.6279 (2016): 1343-1346]. FIG. 6 shows SPR sensograms of the kinetic analysis performed on the various GP proteins. Experiments were performed with Fab114 diluted at the desired concentrations in the range 7.8-250 nM and injected over the functionalized CM5 chip surface. The kinetics were fitted with the Biacore evaluation software. In the plots in FIG. 6 the dotted lines represent the actual measured curves while the solid lines represent the fitting of the kinetics data. KD values—reported in FIG. 7—are in the same order of magnitude for all the GP tested, showing that Fab114 has similar affinity for the for the four GP variants. This is consistent with the available information on mAb114, which typically binds within the GP chalice, perpendicular to the viral membrane, making contact with the glycan cap and the GP1 subunit even after cathepsin cleavage [Corti, Davide, et al. “Protective monotherapy against lethal Ebola virus infection by a potently neutralizing antibody.” Science (2016): aad5224; Misasi, John, et al. “Structural and molecular basis for Ebola virus neutralization by protective human antibodies.” Science 351.6279 (2016): 1343-1346].

### Example 4

### Immunological Profiling by ELISA and Neutralization Assay

Sandwich ELISA with mAbs

96-well Nunc MaxiSorp plates (code 442404, Thermo Scientific Nunc) were coated with a monoclonal rabbit chimeric antibody derived from a human survivor IgG KZ52 (code Ab 00690-23.0, Absolute Antibody) by incubating overnight at 4° C. with 50 μl/well of antibody at 2 μg/ml in 10 mM of phosphate buffer pH 7.4 (PBS 1×, CHUV). After removal of the coating solution, the coated plates were blocked with 300 μl/well of PBS containing 3% milk powder. GP ΔTM-X-HIS, GP ΔTM-ΔMUC-X-HIS, GP ΔTM-ΔMUC-T4-X-HIS, and GP ΔTM-ΔMUC-GCN4-X-HIS were diluted at 4 μg/ml in PBS containing 1.5% milk powder and 0.05% Tween 20 (experimental buffer), and samples were dispensed onto the coated wells (50 μl/well). Recombinant GPs were detected with: the human anti-GP mAb KZ52 (code 0260-001, IBT BioServices); four murine mAbs produced by CEA (provided by Dr. Laurent Bellanger, French Alternative Energies and Atomic Energy Commission, CEA, France) (EZP01S, EZP08S, EZP16S, EZP35S, all tested for the recognition of pseudo types Ebola virus in the in vitro neutralization assays developed in house); the serum of an Ebola survivor (“HUG”) provided by the Hôpitaux Universitaires de Genève [De Santis, Olga, et al. “Safety and immunogenicity of a chimpanzee adenovirus-vectored Ebola vaccine in healthy adults: a randomized, double-blind, placebo-controlled, dose-finding, phase 1/2a study.” The Lancet infectious diseases 16.3 (2016): 311-320.]. Detection antibodies appropriately diluted in experimental buffer (HUG serum at 1/3000 dilution, all the other mAbs at 0.8 μg/ml) were added to the plate (50 μl/well). Horseradish peroxidase (HRP)-conjugated goat anti-human (code 62-8420, Invitrogen) or anti-mouse (code A0412, Sigma) IgG were then used, according to detection antibodies specificity, as second labeled antibody (diluted 1:1000 in experimental buffer, 50 μl/well). Each incubation step was performed for 1 h at room temperature. After the last incubation step, TMB Substrate Reagent Set (code 555214, BD Biosciences) was added to the plates for development (50 μl/well) and the color reaction was blocked after 7 min incubation at room temperature by addition of 0.2 M sulfuric acid (50 μl/well). Absorbance values at 450 nm and 630 nm were determined on a Tecan Infinite® 200 PRO microplate reader. After each incubation step, plates were washed three times with PBS containing 0.05% Tween 20 (wash buffer) using an automated wash station (Tecan HydroSpeed™) to remove unbound antigen and/or antibody.

In Vitro Inhibition Assays with mAbs

In vitro inhibition assays are based on the use of murine leukemia virus (MLV)-derived retroviral pseudotype expressing envelope proteins of desired viruses. To produce MLV-EBOV pseudotypes, three plasmids were co transfected transiently in Human Embryonic Kidney (HEK) 293T cells using polyethylenimine. Used plasmids comprised one encoding Gag Pol proteins from MLV; another the green fluorescent protein (GFP) with a Ψ sequence as an encapsidation signal; and the last one encoding the glycoprotein precursor (GP) of Zaire EBOV (ZEBOV) subtype. ZEBOV GP1 sequence was deleted of its mucin domain. Transfected cell supernatants were harvested and clarified, 48 h post-transfection, and MLV-ZEBOV ΔmucGP pseudotypes were concentrated using centrifugation on a sucrose cushion. They were further purified through an ultracentrifugation (Optima XPN80, Beckman) on continuous sucrose gradient. Purified pseudotypes were then titrated (transducing units, TU/ml) onto VeroE6 cells. The GFP positive cells (i.e. infected cells) were quantified using FACS analysis (FACSCalibur™, Becton Dikinson).

Mouse monoclonal antibodies (mAbs) were developed by CEA using mice immunized with MLV-ZEBOV ΔmucGP pseudotypes by lymphocyte fusion with myeloma cells and cloning, according to Kohler and Milstein [Köhler, G., Milstein, C. (1975). “Continuous cultures of fused cells secreting antibody of predefined specificity.” Nature 256:495-497.]. Their specificity and neutralizing activity were assessed on native ZEBOV viruses in a BSL-4.

In vitro assays were routinely performed in 96-well plates using MLV-ZEBOV ΔmucGP pseudotypes and mAbs to evaluate neutralizing potential of mAbs. Succinctly, a standard quantity of MLV-ZEBOV ΔmucGP pseudotypes was incubated with a standard quantity of mAbs under agitation. Then, the mixture was deposited on Vero cells. 48 h post-infection, cells were fixed with paraformaldehyde and analyzed by flow cytometry to assess GFP fluorescence. To study interactions between recombinant GP and mAbs, neutralizing mAbs (EZP01S, EZP16S, and EZP35S) were pre-incubated at two different concentrations (1 and 10 μg/ml) with various concentrations of GP ΔTM-X-HIS, GP ΔTM-ΔMUC-X-HIS, GP ΔTM-ΔMUC-T4-X-HIS, or GP ΔTM-ΔMUC-GCN4-X-HIS (10 to 150 μg/ml). The resulting mAb/GP solutions were incubated with pseudotypes, before to be added on VeroE6 cells to evaluate the resulting infection rate by FACS analysis.

In Vitro Inhibition Assays with Human Sera

In vitro assays were routinely performed in 96-well plates using MLV-ZEBOV ΔmucGP pseudotypes (as described in a previous paragraph) and a panel of 10 sera derived from the ChAd3-EBOZ clinical trial volunteers [De Santis, O., et al. (2016). “Safety and immunogenicity of a chimpanzee adenovirus-vectored Ebola vaccine in healthy adults: a randomized, double-blind, placebo-controlled, dose-finding, phase 1/2a study.” The Lancet infectious diseases 16(3): 311-320.] to evaluate neutralizing potential of each serum. Succinctly, a standard quantity of MLV-ZEBOV ΔmucGP pseudotypes was incubated with various dilution of sera (1:10 to 1:640) under agitation. Then, the mixture was deposited on Vero cells. 48 h post-infection, cells were fixed with paraformaldehyde and analyzed by flow cytometry to assess GFP fluorescence.

This assay was set up and performed by the group of Dr. Laurent Bellanger (French Alternative Energies and Atomic Energy Commission, CEA, France).

Direct ELISA with Human Sera

96-well Nunc MaxiSorp plates (code 442404, Thermo Fisher Scientific) were coated overnight at 4° C. with 50 μl/well of GP ΔTM-X-HIS, GP ΔTM-ΔMUC-X-HIS, GP ΔTM-ΔMUC-T4-X-HIS, or GP ΔTM-ΔMUC-GCN4-X-HIS diluted at 0.6 μg mL-1 in 10 mM of phosphate buffer pH 7.4 (PBS 1×, CHUV). After removal of the coating solution, the coated plates were blocked with 150 μl/well of PBS containing 3% milk powder. Recombinant GPs were detected with: selected panel of 10 sera of volunteers from the ChAd3-ZEBOV clinical trial [De Santis, O., et al. (2016). “Safety and immunogenicity of a chimpanzee adenovirus-vectored Ebola vaccine in healthy adults: a randomised, double-blind, placebo-controlled, dose-finding, phase 1/2a study.” The Lancet infectious diseases 16(3): 311-320.], 28 days after vaccination; a panel of 10 anonymized Ebola virus survivors coming from a biobank of plasma samples (study funded by the German Research Foundation, grant #MU 3565/3-1) provided by Dr. Cesar Munoz-Fontela (Bernhard Nocht Institute for Tropical Medicine, BNI™, Germany). Sera were diluted 1:100 in PBS containing 1.5% milk powder and 0.05% Tween 20 (experimental buffer), and dispensed onto the coated wells (50 μl/well). Horseradish peroxidase (HRP)-conjugated goat anti-human (code 62-8420, Invitrogen) IgG were then used, according to detection antibodies specificity, as second labeled antibody (diluted 1:1000 in experimental buffer, 50 μl/well). Each incubation step was performed for 1 h at room temperature. After the last incubation step, TMB Substrate Reagent Set (code 555214, BD Biosciences) was added to the plates for development (50 μl/well) and the color reaction was blocked after 7 min incubation at room temperature by addition of 0.2 M sulfuric acid (50 μl/well). Absorbance values at 450 nm and 630 nm were determined on a Tecan Infinite® 200 PRO microplate reader. After each incubation step, plates were washed three times with PBS containing 0.05% Tween 20 (wash buffer) using an automated wash station (Tecan HydroSpeed™) to remove unbound antigen and/or antibody.

### Competition ELISA

Similarly to what described above, the competition ELISA was performed on a panel of 10 sera derived from the ChAd3-EBOZ clinical trial volunteers and on the panel of 10 Ebola virus survivors. During the blocking procedure, sera at their EC50 dilution (previously calculated in direct ELISA) were incubated for 1 h with the GP ΔTM-ΔMUC-X-HIS monomer or the T4/GCN4 trimers at various concentrations. The resulting inhibited samples were then dispensed onto the coated wells (50 μl/well), and assay was carried on as previously described.

### Direct ELISA for Detection of IgG Subclasses and IgM

The assay was carried out as previously described. Coated recombinant GPs were detected with the panel of 10 sera derived from the ChAd3-EBOZ clinical trial volunteers the panel of 10 Ebola virus survivors. Sera were diluted 1:25 in PBS containing 1.5% milk powder and 0.05% Tween 20 (experimental buffer) and dispensed onto the coated wells (50 μl/well). Biotinylated detection antibodies directed against IgG1 (clone G17-1, BD) or IgG2 (clone HP6014, Sigma) subclasses or IgM (clone G20-127, BD) were appropriately diluted in experimental buffer (1:2000, 1:3000, and 1:2000, respectively) and added to the plate (50 μl/well).

### T-Cell Elispot

T-cell Elispot of volunteers of the ChAd3-EBOZ clinical trial [De Santis, O., et al. (2016). “Safety and immunogenicity of a chimpanzee adenovirus-vectored Ebola vaccine in healthy adults: a randomised, double-blind, placebo-controlled, dose-finding, phase 1/2a study.” The Lancet infectious diseases 16(3): 311-320.]. Methods are described in the relative publication.

### Results

The screening and immunological profiling of the designed immunogens was carried out by ELISA in order to assess the recognition of critical neutralizing epitopes exposed on the designed EBOV GP trimers. The aim was to confirm the structural integrity of the produced proteins and their ability to be recognized by conformational monoclonal antibodies. These evaluations were of fundamental importance in order to assess the native-like conformation of recombinant EBOV GPs and to dissect if epitopes recognized by neutralizing antibodies were available. The possibility to produce a protein-based vaccine based on a near-native candidate highly recognized by antibodies of disease survivors would confer us a great advantage in terms of induction of neutralizing antibodies through vaccination.

In this perspective, recombinant EBOV GPs were profiled with several conformational mAbs, including KZ52 [Lee, Jeffrey E., et al. “Structure of the Ebola virus glycoprotein bound to an antibody from a human survivor.” Nature, 454.7201 (2008): 177.] and a panel of murine neutralizing mAbs produced by CEA. In addition, the serum of an Ebola survivor (“HUG”) was used.

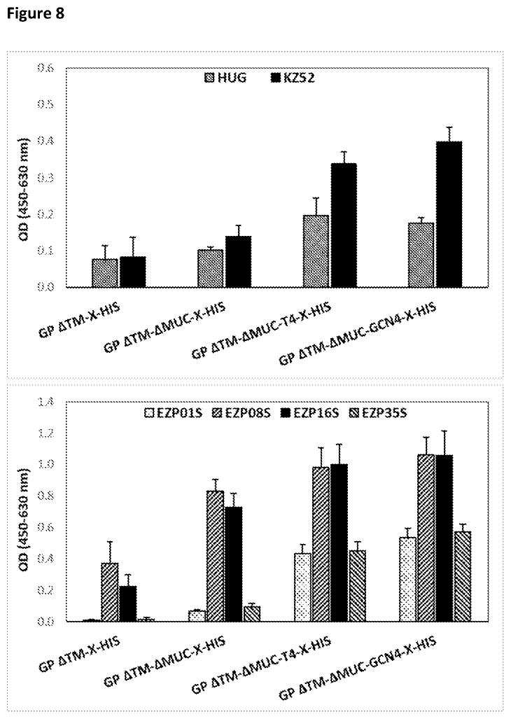

In order to minimize the impact on protein structure introduced by adsorption to the ELISA plate, the immunological profiling of the recombinant proteins produced was performed in a sandwich ELISA configuration, using a monoclonal rabbit chimeric antibody derived from a human survivor IgG KZ52 as capture antibody for the coating. Surprisingly, signal development was obtained with the antibody pair rabbit KZ52/human KZ52 for all the four tested constructs (FIG. 8). Since the epitope recognized by KZ52 is known to be located in the monomeric region of the EBOV GP trimer [Lee, Jeffrey E., et al. “Structure of the Ebola virus glycoprotein bound to an antibody from a human survivor.” Nature 454.7201 (2008): 177], this was identified as a proof of the ability of the monomeric recombinant GPs to rearrange in a dimeric, if not trimeric, configuration when in solution.

The reactivity of the trimers in this assay set-up resulted significantly higher than that of the two monomeric molecules. The performed experiments confirmed that the recombinant proteins were retaining the conformation of the epitope recognized by the conformational and neutralizing mAb KZ52, which is located at the base of the GP chalice. Moreover, the mucin-like domain resulted not to be critical for the retention of the native conformation of the EBOV GP protein, and its removal allowed unmasking of critical neutralizing epitopes located at the base of the chalice. Actually, the recognition of the mucin-deleted protein was higher than that of the native-like protein, both with KZ52 and the HUG serum (FIG. 8).

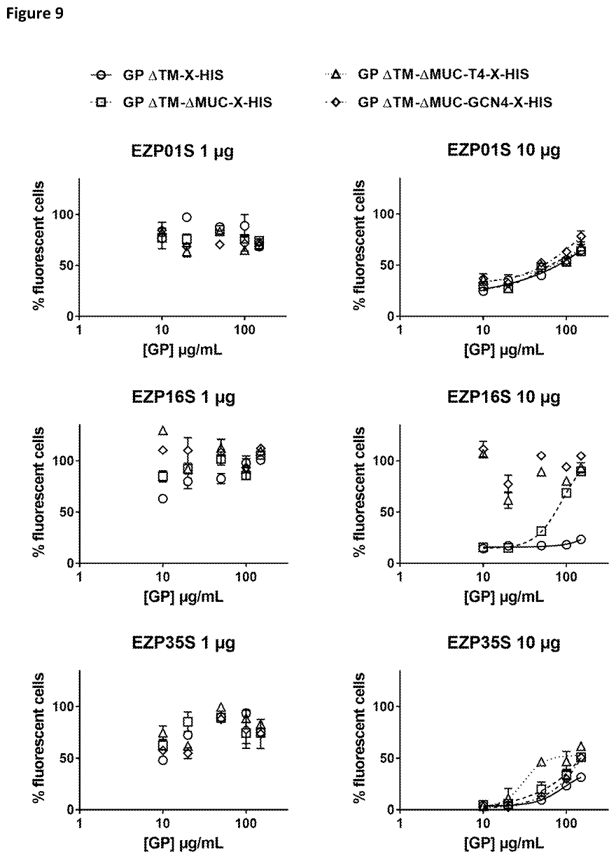

A panel of five conformational mouse mAbs produced by CEA and tested for the recognition of pseudo types Ebola virus in an in vitro neutralization assays was used for further immunological profiling of the antigens. Among these mAbs, listed in FIG. 9, EZP01S, EZP16S, and EZP35S were known for their good neutralizing activity. In all the cases, recognition of the mucin-deleted version of the protein was higher, confirming the initial hypothesis of the mucin-like domain acting as a barrier in preventing access of mAbs to their epitopes. Remarkably, the immunological profiling of the engineered variants showed an increased breadth of reactivity both with the panel of mAbs as well as with the human survivor's serum. These results further validate the design strategy and show that trimeric constructs can be obtained with superior antigenic profiles than those of the wild type protein.

The effect of the GP variants in inhibiting the neutralizing activity of mAbs in an infection assay was assessed on a murine leukemia virus-derived retroviral pseudo type platform. The principle of the test was to evaluate the ability of mAbs in neutralizing the infection of cells made by pseudo-viruses expressing the GP protein. Recognition of the recombinant GPs by neutralizing mAbs was tested (thus inhibiting their inhibition activity on the infection assay). To reach this goal, neutralizing antibodies EZP01S, EZP16S, and EZP35S were tested at two different concentrations (1 and 10 μg/ml), corresponding to an 80% and 0% of infected cells in the pseudo-type assay, against various concentrations of recombinant GPs. As evident from the left panels in FIG. 9, mAbs at the lowest concentrations are captured by the recombinant GPs thus causing a complete cells infection. The main differences are evident in the right panels with the highest mAb concentrations. In this case, mAbs EZP01S and EZP35S showed to be equally sensitive to GP monomers and trimers, suggesting the recognized epitope is similarly accessible in both protein configurations. On the other hand, mAbs EZP16S recognize an epitope that is highly accessible in the trimers, and probably covered by the mucin-like domain given the fact that in presence of the GP ΔTM-X-HIS monomer cell infection is completely inhibited (FIG. 9). Globally, these results support our hypothesis that for induction of broad neutralizing antibodies a trimeric GP antigen is to be preferred.

Day 28 sera derived from ChAd3-EboZ trial volunteers [De Santis, O., et al. (2016). “Safety and immunogenicity of a chimpanzee adenovirus-vectored Ebola vaccine in healthy adults: a randomized, double-blind, placebo-controlled, dose-finding, phase 1/2a study.” The Lancet infectious diseases 16(3): 311-320.] were analyzed by a direct ELISA against GP ΔTM-X-HIS and GP ΔTM-ΔMUC-X-HIS in their native configuration or after a denaturation and reduction treatment. This treatment consisted in an overnight incubation step at 37° C. in presence of guanidinium chloride 6 M and DTT 0.05 M (disulphide bonds reduction), followed by a 1 h incubation at room temperature with N-Ethylmaleimide to block the free cysteine residues. The characterization of the antibody response elicited by the ChAd3-EboZ vaccine showed that 2/3 of the volunteers raised antibodies directed against linear epitopes of the GP protein, which are thought to be less protective than conformational epitopes. This result highlighted the importance of having an antigen with native-like structure, expected to induce or enhance conformational antibodies with a higher chance to be protective. A panel of 10 sera were selected as representative of various types of responses: sera directed preferentially against the native or denatured protein, and sera recognizing preferentially the linear epitopes of the mucin-like domain.

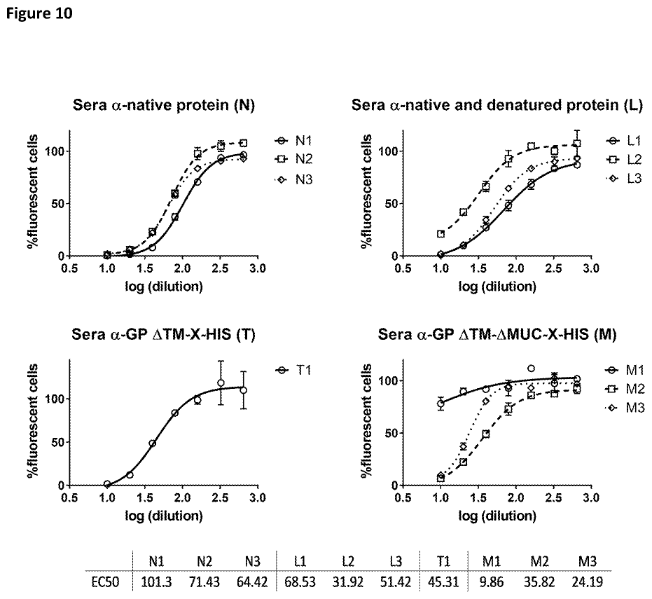

The panel of 10 sera derived from ChAd3-EBOZ vaccinated volunteers was analyzed in the pseudo-virus neutralization assay of Dr. Laurent Bellanger (FIG. 10). The panel was representative of the various types of responses identified: sera directed preferentially against the native or denatured protein, and sera recognizing preferentially the linear epitopes of the mucin-like domain. As a result, the highest affinities for the GP protein expressed on the pseudo-virus surface were found in sera recognizing mainly the native GPs, thus confirming the importance of having a native-like vaccine candidate.

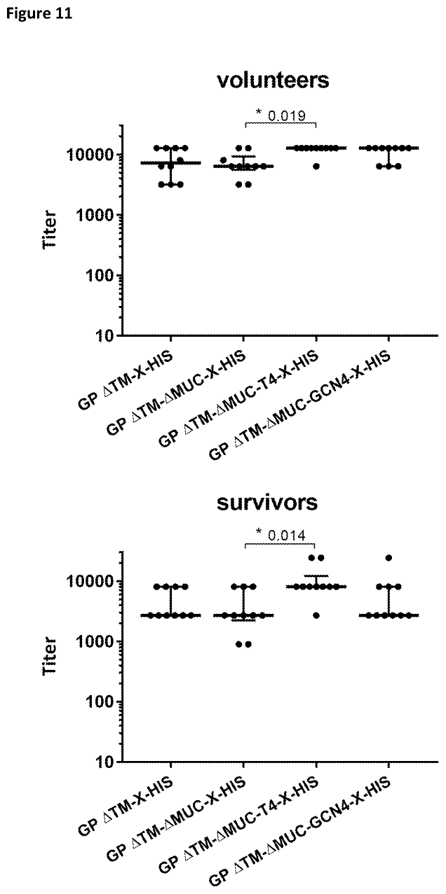

The panel of 10 volunteers' sera was tested in direct ELISA against the recombinant EBOV GPs and the antibody responses were compared with responses of the panel of Ebola survivors (FIG. 11). GP ΔTM-ΔMUC-T4-X-HIS was the most recognized protein by both volunteers and survivors.

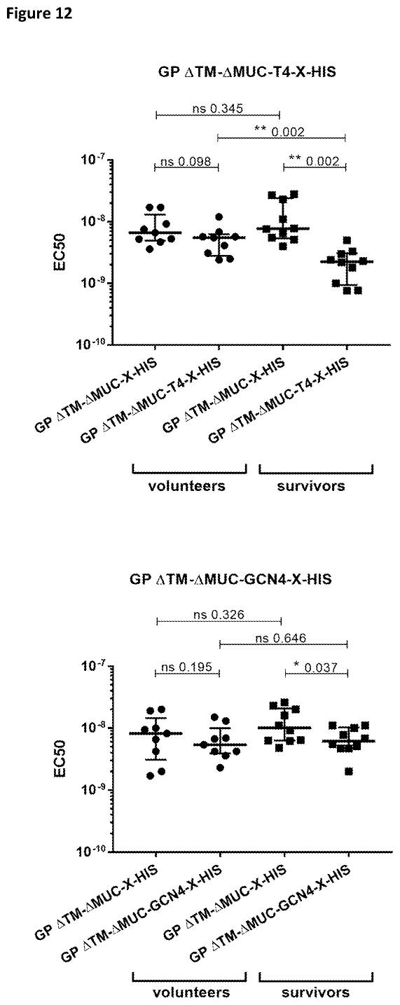

The two panels of 10 volunteers and 10 survivors were compared in a competition ELISA against various concentrations of the monomer GP ΔTM-ΔMUC-X-HIS or the T4 and GCN4 trimers (FIG. 12). Since the panel of survivors showed a higher affinity for T4 trimers, this protein was finally selected as antigen candidate for the Ebola GP-based vaccine.

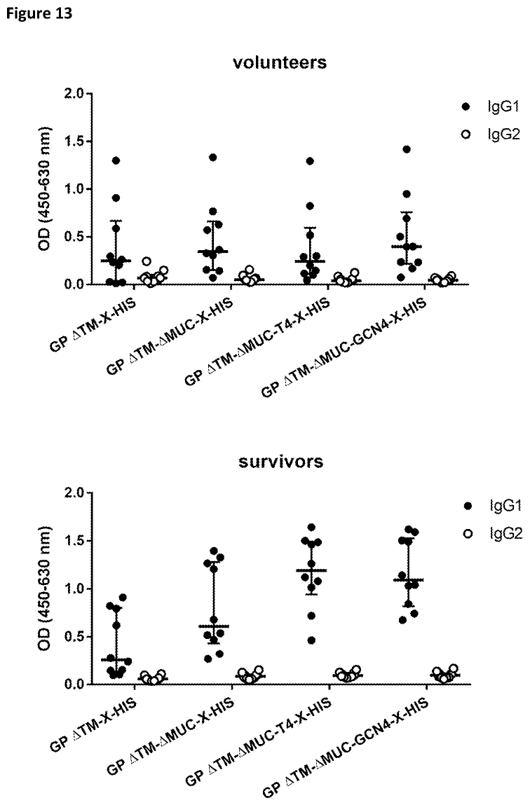

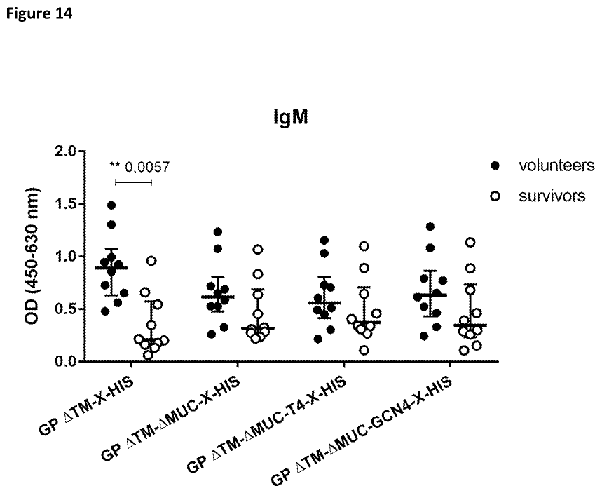

Survivors showed to have a majority of IgG1 antibodies directed against trimers (FIG. 13). The IgG1 subclass is less evident in the volunteers' panel, though being perceived as important for non-recurrence of the virus in long-term survivors [Radinsky, O., et al. (2017). “Sudan ebolavirus long recovered survivors produce GP-specific Abs that are of the IgG1 subclass and preferentially bind FcγRI.” Scientific reports 7.1: 6054.]. Moreover, volunteers show an increased amount of IgM antibodies mainly directed against the mucin-like domain (FIG. 14). This might be typical of vector-based vaccines, since the rVSV-ZEBOV vaccine have been shown to induce low affinity and low-durability IgM [Khurana, S., et al. (2016) “Human antibody repertoire after VSV-Ebola vaccination identifies novel targets and virus-neutralizing IgM antibodies.” Nature medicine 22.12: 1439.].

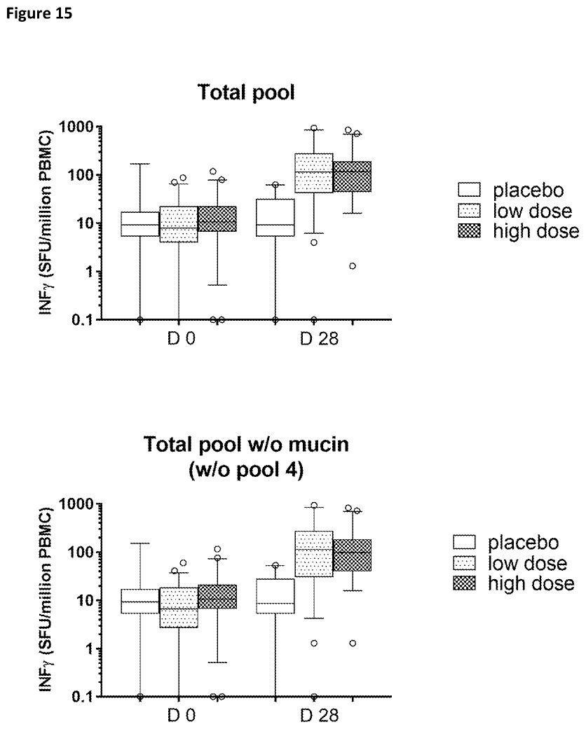

A re-analysis of the T-cell Elispot performed on sera from volunteers of the ChAd3-EBOZ clinical trial highlighted a benefit in terms of immunogenicity for removal of the mucin-like domain from the GP sequence (FIG. 15). T-cells were stimulated with a pool of 15-mers overlapping peptides covering the entire sequence of the GP protein (left graph) or the same pool without the region corresponding to the mucin-like domain. Analysis was performed before vaccination (D0) or 28 days after vaccination (D28) in the placebo group, as well as in the groups of people immunized with the ChAd3-EBOZ vaccine at low dose or high dose.

### Example 5

### Clonal Cell Lines Production and Characterization of GP ΔTM-ΔMUC-T4

### Production and Purification of Recombinant GP1/2 Variants Expressed in Stable Recombinant CHO Cells.

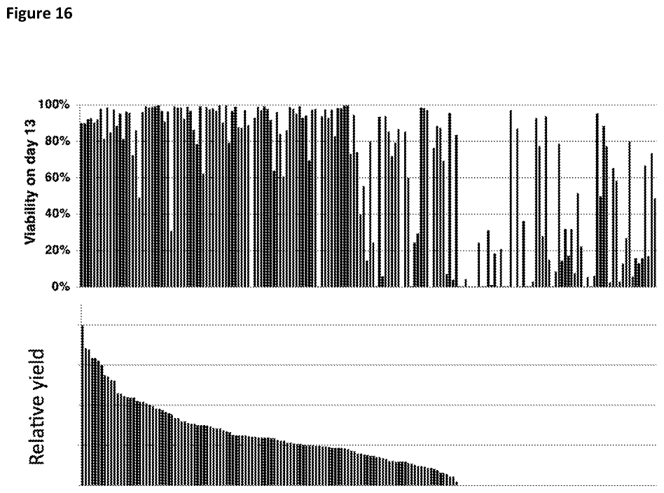

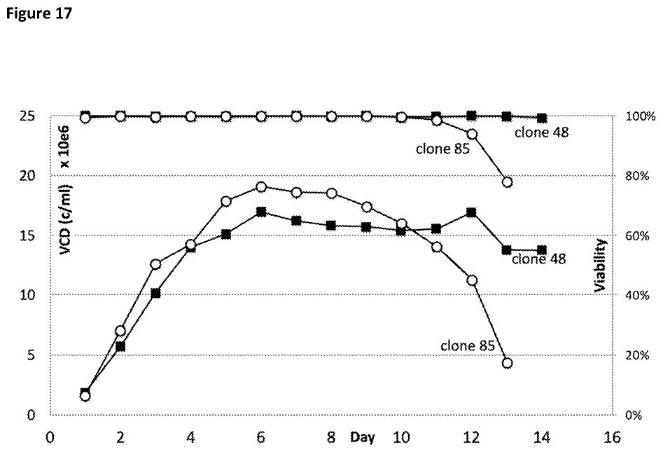

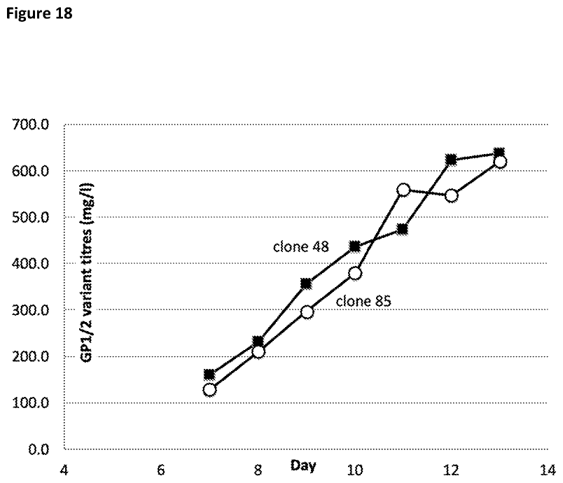

After verification of expression in transient expressions, stable recombinant cell pools, derived from transfections with corresponding expression vectors, were expanded. Production experiments to obtain moderate quantities of research materials were executed with non-cloned cell pools using ExcellGene's proprietary media and process conditions. In each case, productions were done under high density cultures, whereby the production was initiated at a cell density of 0.5×10⁶ cells/ml. Supernatants were harvested after 10-14 days. For the majority of constructs, expression levels of 10s to 100s of mg/l were obtained. Purification was done after clarification and cell removal by buffer exchange and chromatography using a GE Healthcare HiScreen™ NiFF column for His-tagged proteins, following the manufacturer's recommendation for use. For clonally derived cell lines and eventual use in GMP manufacture for clinical use, the construct for the expression of GP ΔTM-ΔMUC-T4 was used. This construct is shown in a simplified diagram in FIG. 3. In FIG. 16 viabilities and ranked productivities of derived clonal cell lines on day 13 are shown. From the leading cell lines, two were eventually chosen (Clones 48 and 85) for further use and the cell culture performance and productivity in fed-batch processes are shown in FIG. 17 and FIG. 18.

For non-His-tag carrying protein variants anionic exchange chromatography was used after clarification of cell culture supernatants and sterile filtration through 0.2 μm. The pH adjusted material (pH 5.0) was loaded on an AIEX column (2× HiSceen Q HP, GE Healthcare) and the column was washed subsequently with 20 mM piperazine, 50 mM NaCl, pH 5.0, followed by an isocratic elution of the column with 20 mM piperazine, 160 mM NaCl, pH 5.0.

A second purification step for non-His tag protein was performed using Hydrophobic interaction chromatography (HIC), using the AIEX eluted product, diluting it 1:1 with 50 mM Na-phosphate, pH 7.0 and adjusting NaCl to 4 M, plus a final pH adjustment to pH 7.0. This material was loaded onto a HiTrap Butyl HP (GE Healthcare) column. The column was washed with 50 mM Na-Phosphate, 4 M NaCl, pH 7.0. and the subsequent elution of purified material occurred through a gradient elution with 50 mM Na-Phosphate, pH 7.0.

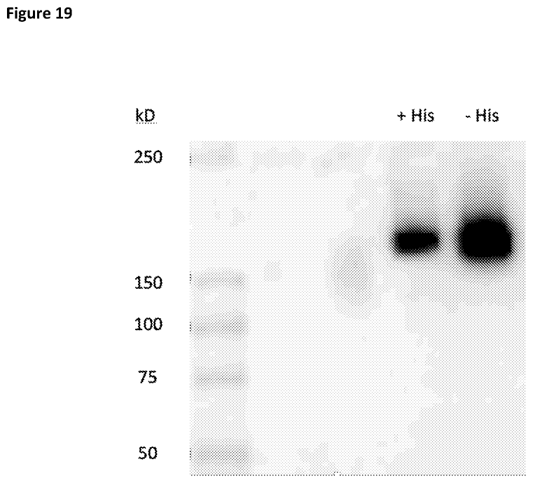

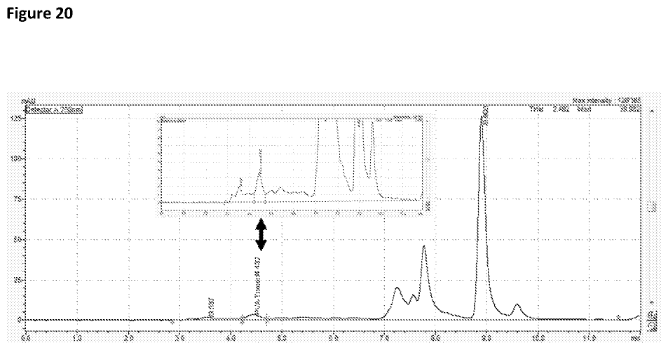

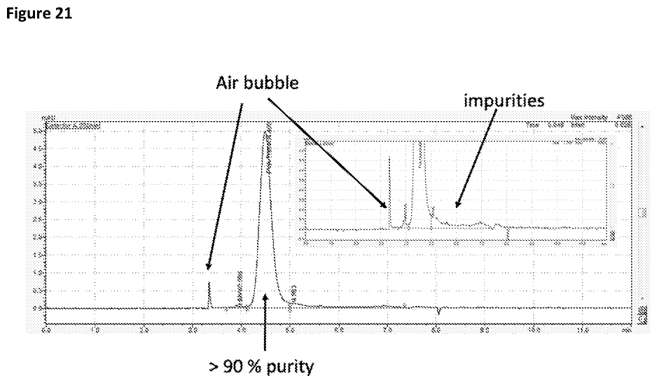

Purified trimer containing proteins with and without a His-tag were analyzed in a Western blot after non-denaturing, native gel electrophoresis. FIG. 19 shows the result, indicating molecular complexes of monomers, as judged by the expected molecule weights. FIG. 20 strengthens the assumption of trimerization, when running AIEX eluates on an analytical size exclusion chromatography column. High purity GP ΔTM-ΔMUC-T4 is shown in FIG. 21. FIGS. 18, 19 and 20 strongly support the secretion of trimers, whether His-tagged or not.

What is claimed is:

1. An isolated polypeptide antigen comprising an Ebola virus glycoprotein (EBOV GP) comprising one or more modifications selected from the group consisting of (a) transmembrane and intracellular tail sequence deletion; (b) mucin region deletion; (c) T4 domain insertion; (d) GCN4 domain insertion; (e) a Factor Xa protease recognition sequence; and (f) a histidine tag sequence.

2. An isolated polypeptide antigen comprising an EBOV GP comprising a transmembrane and intracellular tail sequence deletion, a mucin region deletion, and a T4 domain insertion.

3. An isolated polypeptide antigen comprising an EBOV GP comprising a transmembrane and intracellular tail sequence deletion, a mucin region deletion, and a GCN4 domain insertion.

4. The polypeptide of any one of claims 1-3 capable of eliciting an immunogenic response.

5. The polypeptide of any one of claims 1-4 capable of being bound by antibodies known to bind wild-type EBOV GP.

6. An isolated polypeptide antigen comprising or consisting of an amino acid sequence that is at least 80% identical to a sequence as set out in any one or more of SEQ ID NOs: 1, 3, 5, 7, 9, 11, 13, 15, 17, 19, 21, 23, 25, 27, 29, 31, 33, 35, 37, 39, 41, 43, 45, 47, 49 or 51, or a fragment, analog or derivative thereof, wherein said polypeptide, fragment, analog or derivative is capable of eliciting an immune response specific to the polypeptide antigen.

7. The polypeptide of claim 6 comprising or consisting of an amino acid sequence 43.

8. The polypeptide according to any one of claims 1-7, wherein said polypeptide is cleaved into two subunits that are linked by a disulfide bond, thereby forming a heterodimer.

9. The polypeptide according to claim 8 wherein said heterodimer assembles with two additional heterodimers comprising polypeptides according to any one of claims 1-7, thereby forming a trimeric conformation.

10. A polynucleotide comprising a nucleotide sequence encoding the polypeptide of any one of claims 1-3 and 6.

11. A vector comprising the polynucleotide of claim 10.

12. An expression vector comprising the polynucleotide of claim 10 operably linked to an expression control sequence.

13. A recombinant host cell comprising the vector of claim 11 or the expression vector of claim 12.

14. The recombinant host cell of claim 13, wherein the host cell is:
(i) a eukaryotic cell selected from the group consisting of mammalian, yeast, insect, plant, amphibian and avian cells; or
(ii) a prokaryotic cell.

15. The recombinant host cell of claim 14, wherein the host cell is a Chinese Hamster Ovary (CHO) cell.

16. An antigenic composition comprising a polypeptide according to any one of claims 1-3 and 6, wherein the polypeptide is present in the composition at a concentration of about 0.1-2000 μg/ml, in a pharmaceutically acceptable carrier, diluent, stabilizer, preservative, or adjuvant.

17. A method of producing an immune response to an Ebola virus in a subject, comprising administering to the subject an effective amount of the antigenic composition of claim 16, thereby producing an immune response to a Ebola virus in the subject.

18. A method of preventing a disease or disorder caused by an Ebola virus infection in a subject, comprising administering to the subject an effective amount of the composition of claim 16, thereby preventing a disease or disorder caused by an Ebola virus infection in the subject.

19. A method of immunizing a mammalian subject against an Ebola virus infection comprising administering to the subject an effective amount of the antigenic composition of claim 16, thereby immunizing the subject against an Ebola virus infection.

20. The method of any one of claims 17-19, wherein the administering is intramuscular administration.

21. A method of producing a polypeptide according to any one of claims 1-3 and 6 comprising introducing into a host cell the expression vector of claim 12 under conditions such that the cell produces the polypeptide.

22. The method of claim 21, wherein the host cell is a CHO cell.
